SEQUENCE LISTING (23 sequences)

SEQ ID NO: 1 - DNA, 16894 bp, Histophilus Somni
ctcgaggtttacgataagtgacgaaaactgcggttaataaaccgataaccataccgataaccggaattgccattgccaaagagacttctgctacactggtttttaagccgagtgaagcacctacttgattaatatttttcactaaaacactatcaataaaaattttaccgaaaccgacggggagaagcatataggttgcagtcaatccaaatgtcaagacacaagcaaccgcacgacgatcaacttttagtttattgaaaattgcaaggagcggaggaataacaattggaataaatgcgatgtgtacaggtaataagttttgcgaagaaattgagaataacactagtatggcaagaataaagtatttaaatccagccatcgaattacctgttggcgatttacctaagcgtttaattactttaaaagcaagtagatcggtaatacctgatttagagatggcaatagcaaaagcacctaaaattgcatagttcattgcaacttcagcccccccgcctaaaccgcctgtgaacgcttttattgtttcattaatggttaaatcgctacatagcccggcaactaatgctgagataactaatcccataacaacattaattcgcataagacttaaggcgagtaacacaataatggaaattactactgggttagttaataacataatgttcttccttatggttacttaattttttgaggaaaactcttaatctactcgaaaaaaggagagatttaaagaggaaattagtattatttaatgaaaatgacagttatgataaaacaaagataaaaataagaaagtaattactgcgtaattcattccattcaatagtataaaaactatagtataatcaattttctttttagcacaggaaaggggtatttttttatgaaaaattatgaaaaagacacgatgaaattgctaagaatgatttatactggtattaccagttgtctaaatagcgtatagtataatttgttccaattaaagtggaaggacggcgttttatgccaaaatataaatataacaagataaatatcctattttcatgctgtttactagtttcttttgttgttgcacctttagttgtgcaagcacaagaagatcctttggttacgcaagcaagagaagcagcgaaagaggctaagcctttggttcaagagttattgcatttaaaaaatcaacaacaatttgaaaaagttgagcaaaatttcgagaaagcacagcggtttttggaagataatcgaccaaatcaagaacagatagttgagcaattaaaatcagcaaaagatgtacaaacgatcacgaagatcaccattgattttggtggggaagagatctttttggattttgatgaagtaacaaaacattatttaaaccaaccattatcggcaaaaacggtgtttgcgttgacaaaagagctaacacaagtattgtacaatgccggttatgtaaccagtgctattggtttaaaatcttcaaagatcaaaaacggtgaagtggaatttgtcgtactttggggaaaagtaaatgatattttagtggaagaacagcaggcttctttgttcaaagataaagcaatgctatttgttttgcctaatttaaaaggaaaggtacttcgtatttacgacgtggatcagctgatcgaaattctaaatacgggaaataagacagcaaaggtgaatgtgattgcagcgaatgagaaaagtatgtcaaatttgagcattgagaggcaccgcacttcttatccgcaggttagtttaagcctgaataattccggtgaaggaagtaatgccgaaggtcgaaatcaggcaacattaagcatctcatggagtgatctgttaggaacgaatgaccgttggagtttttcaacgggttatcgtatttataaggatcgtcaagcaaatcggcaacaaaattattccttgtcttatactcaacctttctcattttcgactttggatattaagttatcatattccggttataagaaacagttgagaggaattcatactcacggatccagtggggaaacaaagcaagcgtcatttaagttatcgcacacattattgcgtaataaagacatgattttatctttatatggagaacttgagtttaaaaggcgtcttagctactttggtgatattcgcatagggaaatatcacaataacaaattgaatataggattatcttatgtgaccaatttggggcatggcaaactttatagtgatctgtcttattccaatggactgcggtggtttaatgcaaatcattctgcatataattcacatagagacaaaacattaagattggtttcaggcagtattaactggcagagaccgtttgttttttttaatcgaggaatgagttatcaatttcgtttaggtgcacaatatggatttgatagcttatatggagagaaccagttctcaattggagatgaatatacggtaagaggattcaaaggtggggcaggctcaggtgatcgtggtttctatatttctcaaacagttacgatccctttttaccctcaaaagagttatttatcttacattaacccttttttgggtattgatataggaaaagttcatgcaaaacgaccgcaccatgttgtagatacgtttgcggggtttgcattcggtgtgaaagctcaaataaaatcacttgcattatcattgacttatgctaagcctattaatggagtgggaacttttgaggaaagtaacaagaagtcggttttttatttcacaggttcggtttcgttttgattttttagtttagaggaaggtattatgaataagaattgttataagcttattttcagcaagacaagaggttgtttagttcctgtagcagaatgtattacatcagctgtagatagtggttcatctgattctgttgttgtgtcagaaaaaacagacgaggaagaccggcagggatccattgaagactaccgcttatccaacgtatgtttatccgtaaaaacctttttaaatccggtatcttctgcgttgtgcttaaattggaaaagcgtttctgtattgctgttaagtatggtagcagcaccgaattttgcacagtcggcagaggaagcggcaaaagcggagaaaacgcctaagctaactgaaattcaaaatggtaatgacggtatccagttagagactaagaatcaaaatataggagtaggtgcaggtacaactgaaaataaccatcctactaagctatacaaaacagaaaataacgtcattgttatcgatattgcgaaaccgaatgataaaggaatttcagataaccgtttccaaaaatttaatattccaaacggagcggtttttaaaaataataaagatcaacaacgttctgaattagtaggttatctggaaggtaataagaatctagcagacaaagaagcgaaagttattttaaatcaggtaacgggttcagaacttagtcaaattaaaggggcgttggaaattttaggtacaaaagccgatttggttattgctaaccaacatggtattaacctaaatggagtgcaaactattaatgcgggacgttttgttgctactacgagtaaacttattgatcctaacaagatggaatttgatgttacacaaggaacagtaacaatagatgtcaacgggtttgcaacggataatttaccttacttagatattgttgcgaagaaaattgaacaaaaaggtacgattggaaataaagagaaagaaaagaacaaaacatcggaaacagaaattaccttcattgctgggaaaggtaaaatcaaatataacattgagaatgatggtaagacaaaattagaagtacaaaaagatagtaataccagtcagccttccgataaagaagaagtcgccattactggggcaagtacaggtgcgatgcacggtaaaagcattaaattgatcgtaacggagcaaggtgcgggtgtaaaacacgacggtattattttgtctgaaaatgatattaaaattgaaagcaataaaggggatattgatttaggtgataaactacaagccaaaaatgagatttcattaaataacgccaaaagaattactatagcaaatgagattacggcggataagtctatcacaataaccgcagatgatgttaagttaaaaaataataaagaggcatcagccacagaggaagctaagttaaaaggtaaagggaagttagcctctaaaaaagtaaaagtggaagcaaaaaaatctcttgtgttggatgacgagacaaaagtcgttgcaacggatcttgagcttaaatctcaaacattgactaaccaagggcgtatttacggaaataaagtaaaaattgataccgataaattagtcaataaaaaagaaatttatgcggaagacaatcttgatattacaaccaaaggtaagacagttactgtttctgtaaataaagacaataaacgcaaggcggacgttaaagaagaaaccgttgctgatttagatgttggctttgaaaataccggtaccattgaaagtaaatctaaagctaaattaacttttaaagataatacaagttttgtcagcaagggtaacaagtttataaaagcgaaagatgagttgactattgatgctcaaaatgtggttattagcgaaaatgatgaactacaaactactgcaagacttaccattaatgcggcaggtaatgttgttaacaacggattattagccagcggaaaaacgctgacgattaatgctaaacaggggagtatctataatgaaaaagggattttgggtgctcgtgaacaactgactttatctgctaaaggaaataataaagaaactgagggaaatattattaatggagcggattctctattgcattctgagggtaaaatggaactcgatgcagaaaataccgtttacaacttgggtaatatttttgctaagagcgatcttactgtaaaagctaatgaactgattaatgatgttaaactatccggtagcattactaaaaaatcaccttattcggtgttaaatcgttatcgccgttctgatattgcaagtcatggttggcataataacgactatcgtttatggattaatccaatagaatttgaaaaagcagaagtgaaagtcgaaaaagcaggtttgatccgtgcggaagggaatttcaaattcgaaggtaagaaaggagataatcaacaagatgcaacattgactaaccatggtgtaattaatgttaaaaacactttcgaagcccaaaatgcgaaagttgtaaataatatgaaggcttaccaagccaatttgttgactgaatttttcaaacaaaaacaagatattacatttaattatcagccgagagcccgtctctttttatccgcactttcagggcaagcagagagaaaattcaattctcttgaggagctttttgacggtttattttctgagcaaccaataacaaattccagttcttattatgcggataactcgcaagctgtacatttattagaagagattaaatcgccgactttccaaaaagcaatgactttggtctttggggcaaattggaaaaatgaagaccacaagaaattatcacaacgctggaaagaatttaaagaaaaacaagatgcacattttgattatcgtccaactgataaagccaaaattcttgctcaaagaattaatggcaaaatagatgagttaaaaaatggttcaaccggaggattttctgaaagtgaacgaatcactgtaggacaacataaattcgacttaagtaaagtagagtttagatcggaagtgaatagaaaagaaaatctgaataatagcaatgttgatttatccgcattgtcagatttgttatctattccaaatctttttgtggataacagcgtacagttagataaaacagttgataaaaatattgaaattgatgaggaagatgaatttctattaaaacctcatacaggagaagaacctgatttattgaatgaaaacgaattatcagagaatggaaaattcttagataagttgttgggtgaaattggtgaaaaaacatatattcgagaggtatctgatgattgggagcgagatcccgatgaaccagacgaaccggattataaaaccgaatcaagactagaaacaagagatcgctttgatactttgcctagtgaagtacaagataaattacgccaaaaattcaatgaatataaagaaaaagctcagcagaaacgtcaagctgaagctcttcaagcaaaaaccaaaaatgaacaactacaaagtgacctagaaacaggttataaagaggaagaaaaacgtcaagcgaaaaacgatcttgaaaaacaagcagagttgcaacagcttgatcagcaagaaaaagaaaagcttgcgaaagagaaggagttacaaggcaagattaacgaggaaaaacagcaagaggctttagcgaagcaaaaacaagaacagcaaaaacaagcggatgcaaaagcgaaaattgaagaagaaaaacgtcttgaagaatacagaaaagagctggcgaaagatcatcaaattgaagaagcactttcgaaaaaccaattcttaaaagaggtagatgatacacgtcctaaagtggaaaccgatccgctttacagaacgaaattgcagtatattaaccaagatgaatattttggatctaaatatttcttaaataaagtaggttcatctaccgatgccggtaagaaagtagcggtaattggtgataactatttagaacatcagttaattacaaaatctattgagaagaaagtagataaccatttggcattgaaatatcaagtaaatgatgcacagttggttaaaaaattaatagataactcttattttgaatctaaagagttaggtttgaaagttggggaagcactaacaaaagaacaacaaaatcaacttaaacaagacattgtttggtatgttaaagccaatatcaacaataaagaggttctattaccgcaagtttattttgcaaataaaacattaagagatgctgaaaaattcaaagggcttggtgatgcacttattcgtgctaatgaaattaatttgaaaacaagagatgtgctaaacagcggtacgatttccggtaaaaatattgatattgaagccgaaaataaaattaaaaatagaggtgatattttaagtgaggaaagcactcgtttagttggacacaaaggaattgataataccgcccgctcttttgttaatggaaacggcgatgttgaagttcaacgtgcatcaattagaacagagggacatttacaccttgaagcagatgaagattcggatattaattcaaaaggatcggatattaaaggtaaaacaggttttgtgaaagctcgtaattttaatacgacagatactcacagaactgagcattccgttgaaaaaggtagaattttctctaagaaaggcgagatattgggctatcgtaaggaaagcactcaaaaagcaatttccgttgggtcaaataccgaatttgatcacgtacatttcgctataaaaaatgatgtgaatcaggaaggcagtaagatcaaggcaaaagttgttaccggagttgtacaaggtgattataacaccaaggcaggtcgaaatgctcaacagacggaacgttacattcgcttggatcaagaatattcatccggtcatattagcggtgcagggttcactgtaagccatgaacgtgatagccaaaatggagagaaaacaaatattggtggtgcatcgtctaataccggcaccggttttactttgggcggttctttcagcgaaacgagagaaaaagaaacatcattaactcacaccaacagcgatttgcaggtcgatcatggtattttacatgtattgaaaaaagcggaaatcggtggtgttgatattaacaagcataagttcacaggaaaagcagtcgaggaagatgaagcgaaagctgagcaacaggctaaggcaaaagccgcacctgatgcaacagacaacgcagctcagaaagaggaacctaaatttaaagttctatctcaaagtgaagttgatgatttgatgacagaaaaatctgccaatgatttattcaacaaatataagaaagttaaagaagatgaaggctttgaactttctgcaaaagagattacatcgaataaacaaaaagatgaatatcatcttgattctgaacgttcagtcttgaaattcggtattgaaacggaaggacactcagctattgcagatgctgttagccatgtagcgaaagaaattgtagaggcacaacgtggcgttaagcaagatggtacagttgcgttgcaacacattagcgatgttgcaaatattgttaccggcgaattggtcggcggttcatctaagtttggatttgagcgtaattacgaaacaaataaggttaaggaaacctctgacattcgcacaaaaattgcaggtaatattacgctttcagcccatggtggaaatctacagttgaaaaatgtagaaagcgatgctaacagcaagctaacgcttcaagccaaacgtaatgttgatatacttgatggcgaaacaacaagagaaagtactgaaagacaatctcgtcagaaatttgcatttggtataaattcaggttgtagcgtgatgagcggaggttgtaatggtggcgtatcgggttctgttgatggcaatgaaagtttcacgacagaaaaatctgttacacataacaacagcttgttgagagcgaaaaatctaaaaattgcagcaggtaaagatcttaatttgattagctcaaatattaaagcggatcatttggatttaaatatcaaaggtaaaacgaacattgtatcaaaacaagattccttcgatagactttatagaggttttgattttagtgcttcagcaggtgccgcactttctagttccacacttgtaaaaggtaatggaagttttggggcaggttatactcatgaagttgagaacagaaaacttcttaatcaacaggcaggtattgttgcgaacagaattaccggacaaattaaagatttggatcttgtagcggctcatttcatcaataaagatgaaaatagtggcttccgtgtttcaggaaatgttacgagccaacaacttaatgatagtcatcataaagatggtggttctgtcggcgtttctgtaggtattaacgagcgtggtgcatcttcatttaatgtacgtggtgggcgtgcggagcaaaaacattacgatgcggtacaaaaatcggttatttccggaatcaatcttaaagataataatgtaacgggcgaaattgttgatgatctaagcaaagctaagaccgtaacgagagatgatgtatatgcaagtacacaattcaattttgaagttgcagacttagtggagttgggtgaaaaagcaaaatctaaattacaatctaagtttagcaaagcagtaaataatgatgccgaacagccgactacaaccagaatttcttcagaagatgttgtagaaatggtagataatccgctttacggttctaatgccgacgtacgcaaactaagaactttggatgaggtaggtgaaggttacagcactttaggcgatcaaaatgccaataaaggaagaaaattgcctaacggtagcgatgatatttacagcctcttaggaaaagtgaaagtatctggcgatgagccagtttatgacaaagtgagtgctgaaggtgcttacgatcttttaggcgatagcaacgctaacaagggtagaaccttacgtaacaatagcgatgatctttacagcacagtaggtgatgcaaattcagatatttctcgtatcagatcaaatgtttatgatgagattgcggcaggtccttatagtcttttaggaagaacaaaagcagcagaggaacatatttacgagcaaatcggtgaaggtccttacagtcttttaggtaatggaagtgcggttagaaatagaaccttaggtggtgaaagtaatagcacttacagtacggtaggtgatgcaaattcagatatttctcgtatcagatcaaatgtttatgatgagattgtagcaggtccttatagtcttttaggaaaaccaaaagccgcagaggaacatatttacgaacaaatcggtgaaggtccttacagtcttttaggtaatggaagtgcggttagaaatagaaccttaggtggtgaaagtgatagtccttacagtacggtaggtgatgcaaattcagatatttctcgtatcagatcaaatgtttatgatgagattgtagcaggtccttatagtcttttaggaagaacaaaagcagcagaggaacatatttacgaacaaatcggtgaaggtccttacagtcttttaggtaatggaagtgcggttagaaatagaaccttaggtggtgaaagtgatagtccttacagccttttaggtggtgaagggacgagaaataaagttctagcagatactattgagagcatttacagtaccttgtcccgaccgcaggcatcttctaaccttgaaatggtggataatccgctgtacgacagtgtgagaagatctgcgagtgatcaattgccggaattaccgacagtgagaaacttgttgaatagcgacacggaagcaggtaatggcacttattcggaaattacttctagaacccgcaatgcgaatgatccgttaccacctcttccaaatgagtttagaactcgtttaagtcagggtgcggatttggcagatcatgtttatgatacgattggaagtatttacagcgtattgtccaaaccaaaagcatcttctaaccttgaaatggtggataatccgctgtacggcagtgtgagaagagcggctggtgatcaattgccggaattaccgacagtgaaaaccttgttaaacaaagtggaagaagtaggtaatgaaatttattcggaaattacctctaaaactcgcagtgcgaatgatcctctgccggcacttccaaatttccgtttaactcaggaagttgatacggcagatcatatttatgccgacattaatgatgttgtaaatagagcgaataaagcaaaacgtgatttaccggcaacaccggaggctacacctaaagttgcggtagatggtggagattacgctacaattggtgaggtttcgccgttacaaccgagagcatctcgtcagcaaggaagtagcgattatgaggaaattccactaccacaagaaacggcaccgcaaaaaacatcgcctgtaaaacgcacatcggcagagggtgaagatggttatgcgacaattgctgaggtgttacaaccgagagctgcaaaaggtcaagttagtgattatgaaacaattccgttagacgaaccatcgcaagcagcggtgcgtacagaacgctcggcagtagagggagattatgctgaaattacctctccatcaatacagccgagatcggcacgtggtcagtcgggcggtgaagaatttgaaccgtttccatcggaattttcttccgagccacaatcgccgaaacgtgcattaccggcggaaaatgcggttgtaaatgaactgggtaatgaattaaaagctcggttaaaatctaaagaagatcaagcaaatccggcaaaagcagaggtaagcgagccgatttatgcgacattggataaaagtccggaagggcttgcgagagcgaaagcaaaaggtgatgaagcggcagcagcgaatccgattgtaaaaacgagagtggaagatgatgtagcaccggaattaccagcacgtccatcaaatttgagcgattcaataagcaatgagacaattgcggaaaatggacaatccgttgcgttaggtacaccaaaaagtgcggtggctgaaagtaatcgcaataataacggtaatcagaagctccaatcagaaggagcagagggcgtatcgccaaaaacaaaatcagaggataaatcttggtttgcgaaagtaaaagatttcttctttgcgaaatcgaataaatcacaagctaaagaggcaaaatctgaacaggaaactgtttctaagccgaattatgatagtttggaagatgacttaaatctgaaaaatttattggcgttagaagataaacgtggttcttcttttgaagaaaatgtgttgaaaaatcctgaatttttggcagaagcaagagagattgctaagaaatatattcctgaagctacaatcaaacaaatgggtaattctccggagtttgatgaaatattgacagagggagcgaagaaagtagagaaacgtatcaatgatgccttaactttcaagccgtctgttgatgaatttaatgaaattcagggattggtgaaaaatatacaaaaaggaagtgcggtagatgatctcaatgcacaaactttagcgattacagaagctttagcagatacgtcaaaaacaattcaacgtaatccaaaattgaaagaagaagttcaaggtgcgattgaagaatttttgaaaagtagtcagggcaaagagttaactgtagagatgatcgaaaagttaaatcatggattacgtcctgatgaaggtagcgatcgtttgctttacaaaaaggaaaatttaactaaagaaaatgcggtattttccagtccgcaagcatcaaaaattcagttaaatgaaacagtggactttattaaccaagcaatcaagcaaaatgtggaaccaagtgttttagcggggttggtttatcagcgattaattgcgtaccatccgttcgcagaagggaatggtcgaatggctcgagttgtagtaaataaaattttacttgatgcaggttatccgccgtttactaaattcagttctgaatttgaaacgcagatcattccgcaaacaaaagctacggctaaatcagcaacaagtgcggaagttgtgaaagagtttttgactgaattaggtaaaaaatcatctccgcaagagggaggtgcgaataatcaaaacggacaagcgacttcgcctgttacgttgaagtctaaagatgttagtgaagtcgaaaacacacaatccgcagacagtttgaccattaaacagccggaacaaggtaaagcaggtggtcagttaccatctgttccgaaagtggagacttctgtaaatgaagttgcaccgttgtcatctgtaccagcagaactaaaagatgctgctggaggaaataaaaaagcggcagagaaatcagagggtgctacgggtgtagaaaaagaaaaaaccacacttttccaacgagtgaaacaatttttcaccggaagtaagagcggtgcgaaacctgtagcgggagatgagacagcgaataaagtcaattatcaagatttggaagataatttgaacttaaaaggattaatttctttagaagatgatcgaaatgctaattttgaaagtaatgtattgaaaaatgagaaatttttagatgaagcaagagagatttcgaagaaatcaattcctgaagcgacagttaagcaaatgtctcatttacctgaatttgatgatattctcaccgagggagctaagaaagtagaaagtcgtattaataaggcaatcacattccgcccttctgttgaggagttttcagaaattcaagatttggtgaaaacgttaccgaaaacaaaggttatagaggatctttcaacaaaaacaaatgaaatcacagaagctttagctgcgacatcgaaaaccattcaacgtacaccggagttgaaagaacagttgaagacagcaatagaggatttcttacaaaacagtcaaggcaaacctttgacagtgcagatgatcgagaatcttaatcacggattacgtccggatgagggagaaggtcgtttactttataaaaaagagaatttaaccaaagaaaatgcggtattttctagtcccgaagcggcaaaaattcaattagcggaaacggttgattttatcaatcgagcgaaaaatgaagggattgagccgagtgtggttggggcattagtttatcagcgattgattgcttatcacccatttgcagaaggtaatggacgtatggcgagagtcatagtaaataaaattttacttgatgcaggttatccggcatttaccaaatttagtgatgagtttgaaccgcagattattcctcaaacgaaagcatcaactaaatccgcaacgagcagtgaagtggtagttgagtttttaaaagagttggcaaaaaaaggaagcaaggaagataacgagcagaatttagaaaaaactgaccgcacttctacggacttgacagaaagtgcggtagaaaattcggctgctttgagttccggaacagttagatctgcgacagtttctgaaacagttactgaaacggaacaggcaaaagcgaaaccagttagtgatttggtgagcagtaaagatttagtggaacagcaacgcacggtattgcaacgcattcaggatcaatttcagcctttgaaagtgaaaagcaaaattgatgctgtacgttcttccgttgaagagtttggtggagaagtaagctttaagtttgcccaatctaaaggagaagtttacaaggaaattgtaaaacacattgaaacacaaaatggtgtgtgtgaatcaacttgtgcacattggattgcaaaaaatgttaatccaaccgacgagaatttctttaataccctttatgaaggtggtaaaaaaggacacttgaaaaaagaaactattgattctattaaaaaacttcaaactgagtttattaatagcggtagtgcgactcaacaattcaaattgacagatagttggttgcaagagcagggtgttgttccgaaagagaaaaaagtagcagattttgttcgtcgtgatgaagtatcaggaactgtatcaaaaaatgatgtatcctcattagtaaaagcgattttggatacaggcgatgatactgccggtgttaagaaaatctccattaatcttgagggaggatctcacactgtatctgcggcggttgacggctcaaaagttactttctttgatccgaattttggtgaaatgacatttccaactcatcagcagtttgagaattggttgaagaatgcattttggcaaaaaagtggctacgctggaaagcaagaaggaagacgtttctttaatgtggtcaactacaaaaaaaataattgattaaataatttcttttaatctgacataacttcaaaacgtttctttttagacaggcttgaaactcttcctgcctactaattgggaacgtttttgttttgcttaaaattctaattttacttttaataagaggatggtgttcggtttttgtatatgagcaagcaaaacgacaaatctgataaacataatccataacttgctcaagttgagttttttgattatgtttatttttttgaatttcttgaaaaagtgtagcgttgtattgtgccggaaaatgtttgataaaaaaattgtacattacattagacataataagttcttgaagttaagtcaaagattaagagaaaattccttgttattttcaacaaaaatataatagactattgtccgacttagtcggggtgctgataacagctgagataatacccgtgaacctgatacagttaatactgacgtaggaaactaatggtcttactcctttctttttccaatcaagtcaatattacttatcatgctgtgtttattcaaacaggagaaaagtaatgaacttcaaaaccactctaatttttaccgcactttctgcgatgtctatttgctcttttgctcaagcggaagcgaaatcggcacaaggcttaaccgtttatacttataaaagtttttcctccgattggggggcaggtccaaaggtgaaacaaggatttgagcaacaatatccgcaatgtcgggtaaattatgtgtcctttgacagttcaggtgtattgttcaatcgtctgcgtttagaaggtaagaaaaccaaagcggatattgtactaggcttaagtaatcatttactacaagaggcaattaagtctgaattatttagtgagcataaagttgatttaaatagactttctttaccgatttcttggcaagatcgacgtttcttaccttatgattttgggcattatgcttttatttatgataaaactaaattagtaaatccaccgcactctttgcgagaactggttgaacgtcaggatttacgagttatttatcaggatcctcgaacaagcagtgtggggcggggattaatcatttggttaaatttggtgtatccacaacaagaggtagatcgagcttggcagacattagctaaacataccgttactgtcggtaaaggttggacggaaagttatggggcatttttaaaaggtgaagcggatttagtattaagttatagcacctctccactttatcatttattaaatgaacaaaaagatcagtaccaagcgacagattttgctgaaggcggcgttttacaagttgaattagcagccaaaatagcaaacaaagaaaatgcctgtggtgatttatttatggattacttgatttcaccgcaagcacagacacatatcgttaaaaataacgtgatgttatctgtgatttctgaccctattgagccacattatgatgccttaaaacagtatcagctcaaaacaaatgtgtttgacagttccaatatttctgctgagcaactaaaaaaatggattaatcaatggcaagcaagtttggtgaagtaatttgttaaaagcatggcgttatttttccacattaccaatcagctcattaagtggattggtagtgtggttatttattgtttcattttatggtctggtattgagcggtatttttattgttgaaactgattataaattgacggaactgatacgagatgattacatcaagcaagttattttatttagttttggacaggctaccttatcttccttactttctgtttttttgggcttattattcgctcgag

SEQ ID NO: 2 - protein, 586 aa, Histophilus somni
MPKYKYNKINILFSCCLLVSFVVAPLVVQAQEDPLVTQAREAAKEAKPLVQELLHLKNQQQFEKVEQNFEKAQRFLEDNRPNQEQIVEQLKSAKDVQTITKITIDFGGEEIFLDFDEVTKHYLNQPLSAKTVFALTKELTQVLYNAGYVTSAIGLKSSKIKNGEVEFVVLWGKVNDILVEEQQASLFKDKAMLFVLPNLKGKVLRIYDVDQLIEILNTGNKTAKVNVIAANEKSMSNLSIERHRTSYPQVSLSLNNSGEGSNAEGRNQATLSISWSDLLGTNDRWSFSTGYRIYKDRQANRQQNYSLSYTQPFSFSTLDIKLSYSGYKKQLRGIHTHGSSGETKQASFKLSHTLLRNKDMILSLYGELEFKRRLSYFGDIRIGKYHNNKLNIGLSYVTNLGHGKLYSDLSYSNGLRWFNANHSAYNSHRDKTLRLVSGSINWQRPFVFFNRGMSYQFRLGAQYGFDSLYGENQFSIGDEYTVRGFKGGAGSGDRGFYISQTVTIPFYPQKSYLSYINPFLGIDIGKVHAKRPHHVVDTFAGFAFGVKAQIKSLALSLTYAKPINGVGTFEESNKKSVFYFTGSVSF

SEQ ID NO: 3 - protein, 164 aa, Histophilus somni
MNYAILGAFAIAISKSGITDLLAFKVIKRLGKSPTGNSMAGFKYFILAILVLFSISSQNLLPVHIAFIPIVIPPLLAIFNKLKVDRRAVACVLTFGLTATYMLLPVGFGKIFIDSVLVKNINQVGASLGLKTSVAEVSLAMAIPVIGMVIGLLTAVFVTYRKPR

SEQ ID NO: 4 - protein, 4095 aa, Histophilus somni
MNKNCYKLIFSKTRGCLVPVAECITSAVDSGSSDSVVVSEKTDEEDRQGSIEDYRLSNVCLSVKTFLNPVSSALCLNWKSVSVLLLSMVAAPNFAQSAEEAAKAEKTPKLTEIQNGNDGIQLETKNQNIGVGAGTTENNHPTKLYKTENNVIVIDIAKPNDKGISDNRFQKFNIPNGAVFKNNKDQQRSELVGYLEGNKNLADKEAKVILNQVTGSELSQIKGALEILGTKADLVIANQHGINLNGVQTINAGRFVATTSKLIDPNKMEFDVTQGTVTIDVNGFATDNLPYLDIVAKKIEQKGTIGNKEKEKNKTSETEITFIAGKGKIKYNIENDGKTKLEVQKDSNTSQPSDKEEVAITGASTGAMHGKSIKLIVTEQGAGVKHDGIILSENDIKIESNKGDIDLGDKLQAKNEISLNNAKRITIANEITADKSITITADDVKLKNNKEASATEEAKLKGKGKLASKKVKVEAKKSLVLDDETKVVATDLELKSQTLTNQGRIYGNKVKIDTDKLVNKKEIYAEDNLDITTKGKTVTVSVNKDNKRKADVKEETVADLDVGFENTGTIESKSKAKLTFKDNTSFVSKGNKFIKAKDELTIDAQNVVISENDELQTTARLTINAAGNVVNNGLLASGKTLTINAKQGSIYNEKGILGAREQLTLSAKGNNKETEGNIINGADSLLHSEGKMELDAENTVYNLGNIFAKSDLTVKANELINDVKLSGSITKKSPYSVLNRYRRSDIASHGWHNNDYRLWINPIEFEKAEVKVEKAGLIRAEGNFKFEGKKGDNQQDATLTNHGVINVKNTFEAQNAKVVNNMKAYQANLLTEFFKQKQDITFNYQPRARLFLSALSGQAERKFNSLEELFDGLFSEQPITNSSSYYADNSQAVHLLEEIKSPTFQKAMTLVFGANWKNEDHKKLSQRWKEFKEKQDAHFDYRPTDKAKILAQRINGKIDELKNGSTGGFSESERITVGQHKFDLSKVEFRSEVNRKENLNNSNVDLSALSDLLSIPNLFVDNSVQLDKTVDKNIEIDEEDEFLLKPHTGEEPDLLNENELSENGKFLDKLLGEIGEKTYIREVSDDWERDPDEPDEPDYKTESRLETRDRFDTLPSEVQDKLRQKFNEYKEKAQQKRQAEALQAKTKNEQLQSDLETGYKEEEKRQAKNDLEKQAELQQLDQQEKEKLAKEKELQGKINEEKQQEALAKQKQEQQKQADAKAKIEEEKRLEEYRKELAKDHQIEEALSKNQFLKEVDDTRPKVETDPLYRTKLQYINQDEYFGSKYFLNKVGSSTDAGKKVAVIGDNYLEHQLITKSIEKKVDNHLALKYQVNDAQLVKKLIDNSYFESKELGLKVGEALTKEQQNQLKQDIVWYVKANINNKEVLLPQVYFANKTLRDAEKFKGLGDALIRANEINLKTRDVLNSGTISGKNIDIEAENKIKNRGDILSEESTRLVGHKGIDNTARSFVNGNGDVEVQRASIRTEGHLHLEADEDSDINSKGSDIKGKTGFVKARNFNTTDTHRTEHSVEKGRIFSKKGEILGYRKESTQKAISVGSNTEFDHVHFAIKNDVNQEGSKIKAKVVTGVVQGDYNTKAGRNAQQTERYIRLDQEYSSGHISGAGFTVSHERDSQNGEKTNIGGASSNTGTGFTLGGSFSETREKETSLTHTNSDLQVDHGILHVLKKAEIGGVDINKHKFTGKAVEEDEAKAEQQAKAKAAPDATDNAAQKEEPKFKVLSQSEVDDLMTEKSANDLFNKYKKVKEDEGFELSAKEITSNKQKDEYHLDSERSVLKFGIETEGHSAIADAVSHVAKEIVEAQRGVKQDGTVALQHISDVANIVTGELVGGSSKFGFERNYETNKVKETSDIRTKIAGNITLSAHGGNLQLKNVESDANSKLTLQAKRNVDILDGETTRESTERQSRQKFAFGINSGCSVMSGGCNGGVSGSVDGNESFTTEKSVTHNNSLLRAKNLKIAAGKDLNLISSNIKADHLDLNIKGKTNIVSKQDSFDRLYRGFDFSASAGAALSSSTLVKGNGSFGAGYTHEVENRKLLNQQAGIVANRITGQIKDLDLVAAHFINKDENSGFRVSGNVTSQQLNDSHHKDGGSVGVSVGINERGASSFNVRGGRAEQKHYDAVQKSVISGINLKDNNVTGEIVDDLSKAKTVTRDDVYASTQFNFEVADLVELGEKAKSKLQSKFSKAVNNDAEQPTTTRISSEDVVEMVDNPLYGSNADVRKLRTLDEVGEGYSTLGDQNANKGRKLPNGSDDIYSLLGKVKVSGDEPVYDKVSAEGAYDLLGDSNANKGRTLRNNSDDLYSTVGDANSDISRIRSNVYDEIAAGPYSLLGRTKAAEEHIYEQIGEGPYSLLGNGSAVRNRTLGGESNSTYSTVGDANSDISRIRSNVYDEIVAGPYSLLGKPKAAEEHIYEQIGEGPYSLLGNGSAVRNRTLGGESDSPYSTVGDANSDISRIRSNVYDEIVAGPYSLLGRTKAAEEHIYEQIGEGPYSLLGNGSAVRNRTLGGESDSPYSLLGGEGTRNKVLADTIESIYSTLSRPQASSNLEMVDNPLYDSVRRSASDQLPELPTVRNLLNSDTEAGNGTYSEITSRTRNANDPLPPLPNEFRTRLSQGADLADHVYDTIGSIYSVLSKPKASSNLEMVDNPLYGSVRRAAGDQLPELPTVKTLLNKVEEVGNEIYSEITSKTRSANDPLPALPNFRLTQEVDTADHIYADINDVVNRANKAKRDLPATPEATPKVAVDGGDYATIGEVSPLQPRASRQQGSSDYEEIPLPQETAPQKTSPVKRTSAEGEDGYATIAEVLQPRAAKGQVSDYETIPLDEPSQAAVRTERSAVEGDYAEITSPSIQPRSARGQSGGEEFEPFPSEFSSEPQSPKRALPAENAVVNELGNELKARLKSKEDQANPAKAEVSEPIYATLDKSPEGLARAKAKGDEAAAANPIVKTRVEDDVAPELPARPSNLSDSISNETIAENGQSVALGTPKSAVAESNRNNNGNQKLQSEGAEGVSPKTKSEDKSWFAKVKDFFFAKSNKSQAKEAKSEQETVSKPNYDSLEDDLNLKNLLALEDKRGSSFEENVLKNPEFLAEAREIAKKYIPEATIKQMGNSPEFDEILTEGAKKVEKRINDALTFKPSVDEFNEIQGLVKNIQKGSAVDDLNAQTLAITEALADTSKTIQRNPKLKEEVQGAIEEFLKSSQGKELTVEMIEKLNHGLRPDEGSDRLLYKKENLTKENAVFSSPQASKIQLNETVDFINQAIKQNVEPSVLAGLVYQRLIAYHPFAEGNGRMARVVVNKILLDAGYPPFTKFSSEFETQIIPQTKATAKSATSAEVVKEFLTELGKKSSPQEGGANNQNGQATSPVTLKSKDVSEVENTQSADSLTIKQPEQGKAGGQLPSVPKVETSVNEVAPLSSVPAELKDAAGGNKKAAEKSEGATGVEKEKTTLFQRVKQFFTGSKSGAKPVAGDETANKVNYQDLEDNLNLKGLISLEDDRNANFESNVLKNEKFLDEAREISKKSIPEATVKQMSHLPEFDDILTEGAKKVESRINKAITFRPSVEEFSEIQDLVKTLPKTKVIEDLSTKTNEITEALAATSKTIQRTPELKEQLKTAIEDFLQNSQGKPLTVQMIENLNHGLRPDEGEGRLLYKKENLTKENAVFSSPEAAKIQLAETVDFINRAKNEGIEPSVVGALVYQRLIAYHPFAEGNGRMARVIVNKILLDAGYPAFTKFSDEFEPQIIPQTKASTKSATSSEVVVEFLKELAKKGSKEDNEQNLEKTDRTSTDLTESAVENSAALSSGTVRSATVSETVTETEQAKAKPVSDLVSSKDLVEQQRTVLQRIQDQFQPLKVKSKIDAVRSSVEEFGGEVSFKFAQSKGEVYKEIVKHIETQNGVCESTCAHWIAKNVNPTDENFFNTLYEGGKKGHLKKETIDSIKKLQTEFINSGSATQQFKLTDSWLQEQGVVPKEKKVADFVRRDEVSGTVSKNDVSSLVKAILDTGDDTAGVKKISINLEGGSHTVSAAVDGSKVTFFDPNFGEMTFPTHQQFENWLKNAFWQKSGYAGKQEGRRFFNVVNYKKNN

SEQ ID NO: 5 - protein, 338 aa, Histophilus somni
MNFKTTLIFTALSAMSICSFAQAEAKSAQGLTVYTYKSFSSDWGAGPKVKQGFEQQYPQCRVNYVSFDSSGVLFNRLRLEGKKTKADIVLGLSNHLLQEAIKSELFSEHKVDLNRLSLPISWQDRRFLPYDFGHYAFIYDKTKLVNPPHSLRELVERQDLRVIYQDPRTSSVGRGLIIWLNLVYPQQEVDRAWQTLAKHTVTVGKGWTESYGAFLKGEADLVLSYSTSPLYHLLNEQKDQYQATDFAEGGVLQVELAAKIANKENACGDLFMDYLISPQAQTHIVKNNVMLSVISDPIEPHYDALKQYQLKTNVFDSSNISAEQLKKWINQWQASLVK

SEQ ID NO: 6 - DNA, 40 bp, Histophilus Somni
cggaattccgatgaataagaattgttataagcttattttc

SEQ ID NO: 7 - DNA, 40 bp, Histophilus somni
gcgtcgacttactcatttgctatagtaattcttttggcgt

SEQ ID NO: 8 - DNA, 38 bp, Histophilus somni
gcggatccattacggcggataagtctatcacaataacc

SEQ ID NO: 9 - DNA, 41 bp, Histophilus somni
gcgtcgacttattcagaaaatcctccggttgaaccattttt

SEQ ID NO: 10 - DNA, 40 bp, Histophilus somni
gcgcggatccagtgaacgaatcactgtaggacaacataaa

SEQ ID NO: 11 - DNA, 40 bp, Histophilus somni
gcgcgtcgactcaagctttcacaaaacctgttttaccttt

SEQ ID NO: 12 - DNA, 38 bp, Histophilus somni
gcggatcccgtaattttaatacgacagatactcacaga

SEQ ID NO: 13 - DNA, 41 bp, Histophilus somni
gcgtcgacttatttaatttgtccggtaattctgttcgcaac

SEQ ID NO: 14 - DNA, 38 bp, Histophilus somni
gcggatccaaagatttggatcttgtagcggctcatttc

SEQ ID NO: 15 - DNA, 47 bp, Histophilus somni
gcgtcgacttaggcataaatatgatctgccgtatcaacttcctgagt

SEQ ID NO: 16 - DNA, 38 bp, Histophilus somni
gcggatccgacattaatgatgttgtaaatagagcgaat

SEQ ID NO: 17 - DNA, 41 bp, Histophilus somni
gcgtcgacttattcttctttcaattttggattacgttgaat

SEQ ID NO: 18 - DNA, 30 bp, Histophilus somni
agctggatccagtgaacgaatcactgtagg

SEQ ID NO: 19 - DNA, 34 bp, Histophilus somni
agctgaattcgaagctttcacaaaacctgtttta

SEQ ID NO: 20 - DNA, 29 bp, Histophilus somni
agctggatccgatttggatcttgtagcgg

SEQ ID NO: 21 - DNA, 32 bp, Histophilus somni
agctgaattcgaggcataaatatgatctgccg

SEQ ID NO: 22 - DNA, 34 bp, Histophilus somni
agctggatccatcgaaaagttaaatcatggatta

SEQ ID NO: 23 - DNA, 36 bp, Histophilus somni
agctgaattcagattattttttttgtagttgaccac

(19) United States
(12) Patent Application Publication
(10) Pub. No.: US 2010/0008942 A1
(43) Pub. Date: Jan. 14, 2010

(54) Histophilus Somni Polynucleotides, Polypeptides and Methods of Use

(76) Inventors: Lynette Corbeil, San Diego, CA (US); Jack Dixon, La Jolla, CA (US); Yuichi Tagawa, Ibaraki (JP)

Correspondence Address: SPENCER FANE BRITT & BROWNE LLP, 1 NORTH BRENTWOOD BLVD., SUITE 1000, ST. LOUIS, MO, 63105-3925, US

(21) Appl. No.: 12/442,095

(22) PCT Filed: Sep. 19, 2007
(86) PCT No.: PCT/US07/78949

§ 371 Date: Jul. 8, 2009

Related U.S. Application Data
(60) Provisional application No. 60/826,205, filed on Sep. 19, 2006.

(51) Int. Cl. A61K 39/02; A61K 31/7088; A61P 31/04; A61P 37/04

(52) U.S. Cl. 424/190.1; 514/44 R

(57) ABSTRACT

This invention provides, among other things, protein, polypeptides, and fragments thereof, derived from bacteria Histophilus somni. Also provided are nucleic acids encoding for such proteins, polypeptides, and/or fragments, as well as nucleic acids complementary thereto. Additionally, this invention provides antibodies which bind to the proteins, polypeptides, and/or fragments. This invention further provides expression vectors useful for making the proteins, polypeptides, fragments, and/or nucleic acids, for use as vaccines, diagnostic reagents, immunogenic compositions, and the like. Methods of making the compositions and methods of treatment with the compositions are also provided. This invention also provides methods of detecting the proteins, polypeptides, fragments, variants and/or nucleic acids as well as detecting antibodies against the proteins, polypeptides, variants and fragments.

### CROSS-REFERENCE TO RELATED APPLICATIONS

This application claims priority from U.S. Provisional Application Ser. No. 60/826,205 filed on Sep. 19, 2006, which is incorporated herein by reference in its entirety.

### STATEMENT REGARDING FEDERALLY SPONSORED RESEARCH OR DEVELOPMENT

This invention was made in part with Government support under National Institutes of Health Grant No. DK 188049, United States Department of Agriculture Grant No. 98-35204-6733 (Subcontract No. 2002-35204-11673) and United States Department of Agriculture Grant No. 2005-35204-16257. The Government has certain rights in the invention.

### INCORPORATION-BY-REFERENCE OF MATERIAL SUBMITTED ON A COMPACT DISC

The Sequence Listing, which is a part of the present disclosure, includes a computer file “10100-0082_ST25.txt” generated by U.S. Patent & Trademark Office PatentIn Version 3.4 software comprising nucleotide and/or amino acid sequences of the present invention. The subject matter of the Sequence Listing is incorporated herein by reference in its entirety.

### FIELD

This invention relates to antigens from the bacterial species Histophilus somni (also known as Haemophilus somnus).

### INTRODUCTION

Bacterial disease in livestock such as cattle, sheep, and bison, poses a serious problem for the livestock industry. In particular, livestock afflicted by diseases caused by Histophilus somni (H. somni; also known as Haemophilus somnus), such as septicemia, thrombotic meningoencephalitis, pneumonia or reproductive diseases, can be particularly devastating to the livestock industry. Prevention of diseases is a desired remedy to these threats to the livestock than intervention. Vaccination of the livestock is the only preventive method which may offer long-term protection through immunity.

Current vaccines are useful for prevention of H. somni septicemia and thrombotic meningoencephalitis but data on efficacy for prevention of pneumonia or reproductive failure is less convincing (Corbeil et al., 1995). In addition, adverse reactions are sometimes a problem with commercially available vaccines due to high levels of endotoxin in the vaccines. Antibiotic therapy (or even prevention) is often effective but resistant strains are a problem. Prevention by vaccination is more attractive.

Current diagnostic tools also pose a problem because many animals are asymptomatic carriers, thus positive cultures do not necessarily mean that the cause of disease has been identified. Serologic assays are not very specific so many false positives result. Accordingly, it is an object of the invention to overcome these and other problems associated with the related art.

A certain amount of sequence data is available for H. somni genes and proteins (e.g., J. Challacombe et al. Complete Genome Sequence of Haemophilus somnus (Histophilus somni) Strain 129Pt and Comparison to Haemophilus ducreyi 35000HP and Haemophilus influenzae Rd^{▾}. J. Bacteriol. 189(5):1890-1898 (2007); GenBank Accession No. AACJ00000000 a sequence for H. somni 129Pt; ibpA, ibpB and tbpA sequences having GenBank Accession No. AB087258, but are not complete. In addition, a Dissertation of Shivakumara Swamy Siddaramappa available at (with limited accessibility) http://scholar.lib.vt.edu/theses/available/etd-05022007-150407/ provides a sequence for H. somni virulent strain 2336, but this is not complete.

Providing additional sequences could provide an opportunity to identify secreted or surface-exposed proteins that are presumed targets for the immune system and which are not antigenically variable. It is therefore an object of the invention is to provide H. somni polynucleotide sequences which encode polypeptides that are antigenic or immunogenic.

### SUMMARY

The invention provides polypeptides and polynucleotides comprising the H. somni amino acid sequences disclosed herein, including IbpA, IbpB and TbpA.

The invention also provides proteins comprising sequences homologous (i.e., those having sequence identity) to the H. somni amino acid sequences, including IbpA, IbpB and TbpA, disclosed herein. Depending on the particular sequence, the degree of homology (sequence identity) is preferably greater than 50% (e.g., 60%, 70%, 80%, 90%, 95%, 99% or more). These proteins include mutants and allelic variants of the sequences disclosed in the examples. Typically, 50% identity or more between two proteins is considered to be an indication of functional equivalence. Identity between proteins is preferably determined by the Smith-Waterman homology search algorithm as implemented in MPsrch program (available at http://www.ebi.ac.uk/MPsrch/) using an affine gap search with parameters: gap penalty 12, gap extension penalty 1. Examples of microbes having IbpA polypeptides as defined below include P. multocida, H. ducreyi, Bordetella species (including B. pertussis), Yersinia species, S. pyogenes, and S. agalactiae.

The invention further provides proteins comprising fragments of the H. somni amino acid sequences, including IbpA, IbpB and TbpA, disclosed herein. The fragments should comprise at least n consecutive amino acids from the sequences and, depending on the particular sequence, n is 7 or more (e.g., 8, 10, 12, 14, 16, 18, 20 or more). In various embodiments, the fragments comprise an epitope from the sequence.

The proteins of the invention can be prepared by various means (e.g., recombinant expression, purification from cell culture, chemical synthesis, etc.) and in various forms (e.g., native, fusions, etc.). They are preferably prepared in substantially pure or isolated form (i.e., substantially free from other H. somni host cell proteins).

According to a further aspect, the invention provides antibodies which bind to these proteins. These may be polyclonal or monoclonal and may be produced by any suitable means.

According to a further aspect, the invention provides a polynucleotide comprising the H. somni nucleotide sequences, including IbpA, IbpB and TbpA, disclosed herein.

According to a further aspect, the invention comprises nucleic acids having sequence identity of greater than 50% (e.g., 60%, 70%, 80%, 90%, 95%, 99% or more) to the nucleic acid sequences herein.

According to a further aspect, the invention comprises nucleic acid that hybridizes to the sequences provided herein.

Polynucleotides comprising fragments of these sequences are also provided. These should comprise at least n consecutive nucleotides from the H. somni sequences, including ibpA, ibpB and tbpA, and depending on the particular sequence, n is 10 or more (e.g., 12, 14, 15, 18, 20, 25, 30, 35, 40 or more).

According to a further aspect, the invention provides nucleic acid encoding the proteins and protein fragments of the invention.

It should also be appreciated that the invention provides nucleic acid comprising sequences complementary to those described above (e.g., for probing purposes).

Polynucleotides of the invention can be prepared in many ways (e.g., by chemical synthesis, in part or in whole, from genomic or DNA libraries, from the organism itself, etc.) and can take various forms (e.g., single stranded, double stranded, vectors, probes, etc.).

According to a further aspect, the invention provides vectors comprising nucleotide sequences of the invention (e.g., expression vectors) and host cells transformed with such vectors.

According to a further aspect, the invention provides compositions comprising protein, antibody, and/or nucleic acid according to the invention. These compositions may be suitable as vaccines, for instance, or as diagnostic reagents or as immunogenic compositions.

The invention also provides nucleic acid, protein, or antibody according to the invention for use as medicaments (e.g., as vaccines) or as diagnostic reagents. It also provides the use of nucleic acid, protein, or antibody according to the invention in the manufacture of (i) a medicament for treating or preventing infection due to H. somni bacteria (ii) a diagnostic reagent for detecting the presence of H. somni bacteria or of antibodies raised against H. somni bacteria or (iii) for raising antibodies. The H. somni bacteria may be any species or strain, in particular H. somni 2336.

The invention also provides a method of treating an animal, comprising administering to the animal a therapeutically effective amount of polynucleotide, polypeptide, and/or antibody according to the invention.

According to further aspects, the invention provides various processes including a process for producing proteins of the invention is provided, comprising the step of culturing a host cell according to the invention under conditions which induce protein expression.

A process is provided for detecting polynucleotides of the invention comprising the steps of: (a) contacting a nucleic probe according to the invention with a biological sample under hybridizing conditions to form duplexes; and (b) detecting said duplexes.

A process for detecting polypeptides of the invention is provided, comprising the steps of: (a) contacting an antibody according to the invention with a biological sample under conditions suitable for the formation of an antibody-antigen complexes; and (b) detecting said complexes.

In addition, a method for detecting antibodies to the H. somni polypeptides, including IbpA, IbpB and TbpA, is provided.

The polypeptides of the invention can further be combined with other polypeptides and vaccine preparations known to those of skill in the art.

These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1. Plasmids inserts and location of ibpA and ibpB ORFs.

FIG. 2. Functional studies of bovine IgG2 Fc binding to H. somni IgBPs by Western blotting.

FIG. 3. Graphical depiction of H. somni consensus sequences.

FIG. 4. Schematic representation of the similarity of ibpA protein sequence to P. multoicida PfhB2, H. ducreyi LspA1, B. pertussis FhaB and Yersinia spp. YopT.

FIG. 5. Depiction of competitive inhibition of H. somni attachment to bovine pulmonary artery endothelial cells.

FIG. 6. Bovine IgG2 Fc binding to GST-fused truncated ibpA fragments by Western blotting.

FIG. 7. Depiction of vaccination and protection results in bar graph of mice against H. somni septicemia.

FIG. 8. Depiction of vaccination and protection results in bar graph of mice against H. somni septicemia.

FIG. 9. Sequence diagram for H. somni IbpB and IbpA.

FIG. 10. Line graph depicting clinical scores after challenge of vaccinated calves with H. somni.

FIG. 11. Bar graph depicting volume of pneumonic lesions at necropsy, expressed as percentage of lung affected (% lesions).

FIG. 12. Line graphs depicting IgG1 and IgG2 levels after IbpA and IbpA fragment challenge.

FIG. 13. Line graphs depicting IgG1 levels in ELISA after IbpA and IbpA fragment challenge.

FIG. 14. Line graphs depicting IgG2 levels in ELISA after IbpA and IbpA fragment challenge.

FIG. 15. Line graphs depicting IgGE levels in ELISA after IbpA and IbpA fragment challenge.

FIG. 16. Line graphs depicting whole cell antigen levels in ELISA after IbpA and IbpA fragment challenge.

### DETAILED DESCRIPTION

Abbreviations and Definitions

To facilitate understanding of the invention, a number of terms and abbreviations as used herein are defined below as follows:

Immune response activating amount: The term “immune response activating amount” refers to any amount which would activate a humoral and cellular immunity in an animal subject susceptible to H. somni and similar microbe infection.

Protective immune response: The term “protective immune response” can be shown by measuring antibody shown in animals, injecting an antigen, with or without an adjuvant.

ibpA Polynucleotide: By the terms “ibpA polynucleotide,” “ibpA gene,” “ibpA nucleic acid,” or “target,” is meant a native-encoding nucleic acid sequence, e.g., the native ibpA gene (SEQ ID NO: 1) and related fragments (SEQ ID NOs: 6-17). The terms encompass double-stranded DNA, single-stranded DNA, and RNA. The term also includes ibpB polynucleotide and tbpA polynucleotide as such polynucleotides are included in SEQ ID NO: 1.

Isolated: As used herein, the term “isolated,” when referred to a molecule, refers to a molecule that has been identified and separated and/or recovered from a component of its natural environment. Contaminant components of its natural environment are materials that interfere with diagnostic or therapeutic use.

Protein or Polypeptide: As used herein, “protein” or “polypeptide” refers to any peptide-linked chain of amino acids, regardless of length or post-translational modification, e.g., glycosylation or phosphorylation. The term used alone refers to a polypeptide of the invention including IbpA, IbpB and TbpA polypeptides, fragments and variants. An “isolated” polypeptide or protein is one that is substantially separated from other polypeptides in a cell or organism in which the polypeptide naturally occurs (e.g., 30, 40, 50, 60, 70, 80, 90, 95, 96, 97, 98, 99, 100% free of contaminants).

ibpA Polypeptide: By the terms “IbpA protein” or “IbpA polypeptide” refers to an expression product of a ibpA gene such as the native IbpA protein (SEQ ID NO: 2), or a protein that shares at least 65% (but preferably 75, 80, 85, 90, 95, 96, 97, 98, or 99%) amino acid sequence identity with one of the foregoing and displays a functional activity of a native IbpA protein. A “functional activity” of a protein is any activity associated with the physiological function of the protein. For example, functional activities of a native IbpA protein may include H. somni immunoglobulin binding protein activity.

IbpB and TbpA Polypeptide: By the terms “IbpB protein” or “TbpA protein” or “IbpB polypeptide” or “TbpA polypeptide” refers to an expression product of a ibpB or tbpA genes such as the native IbpB protein (SEQ ID NO: 3), TbpA protein (SEQ ID NO: 4), hypothetical protein (SEQ ID NO: 5) or a protein that shares at least 65% (but preferably 75, 80, 85, 90, 95, 96, 97, 98, or 99%) amino acid sequence identity with one of the foregoing and displays a functional activity of a native IbpB or TbpA protein. A “functional activity” of a protein is any activity associated with the physiological function of the protein. For example, functional activities of a native ibpB or tbpA proteins may include secretion of ibpA or thymine binding respectively.

Polynucleotide: The term “polynucleotide” used alone refers to a polynucleotide of the invention encoding a polypeptide including IbpA, IbpB and TbpA polypeptides, fragments and variants.

Polynucleotide Variant: A “polynucleotide variant” refers to any degenerate nucleotide sequence. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence. For example, a variant polynucleotide consisting of 80, 81, 82, 83, 84, 85, 86, 87, 88, 89, 90, 91, 92, 93, 94, 95, 96, 97, 98, and 99% to the polynucleotide consisting of ibpA.

Polynucleotide Fragment: A “polynucleotide fragment” of a ibpA polynucleotide is a portion of a ibpA polynucleotide that is less than full-length and comprises at least a minimum length capable of hybridizing specifically with a native ibpA polynucleotide under stringent hybridization conditions. The length of such a fragment is preferably at least 15 nucleotides, more preferably at least 20 nucleotides, and most preferably at least 30 nucleotides of a native ibpA polynucleotide sequence.

Polypeptide Variant: A “polypeptide variant” refers to a polypeptide of differs in amino acid sequence from the ibpA polypeptide. Generally, differences are limited so that the sequences of the reference polypeptide and the variant are closely similar overall and, in many regions, identical. A variant polypeptide may differ in amino acid sequence by one or more substitutions, additions, deletions in any combination. A substituted or inserted amino acid residue may or may not be one encoded by the genetic code.

Polypeptide Fragment: A “polypeptide fragment” refers to any polypeptide of a portion of a ibpA polypeptide that is less than full-length (e.g., a polypeptide consisting of 5, 10, 15, 20, 30, 40, 50, 75, 100 or more amino acids of a native ibpA protein), and preferably retains at least one functional activity of a native ibpA protein.

Sequence Identity: As used herein, “sequence identity” refers to the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. For example, if 9 positions in a sequence 10 nucleotides in length are identical to the corresponding positions in a second 10-nucleotide sequence, then the two sequences have 90% sequence identity. Percent sequence identity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). One of skill in the art will recognize that P. multocida, H. ducreyi, Bordetella species, Yersinia species, S. pyogenes, and S. agalactiae have sequence identity in IbpA, IbpB and TbpA of greater than 50%.

Vector: As used herein, the term “vector” refers to a nucleic acid molecule capable of transporting another nucleic acid to which it has been linked. One type of preferred vector is a plasmid, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors.”

IbpA-Specific Antibody: By the term “IbpA-specific antibody” refers to an antibody that binds a IbpA protein and displays no substantial binding to other naturally occurring proteins other than those sharing the same antigenic determinants as the IbpA protein. The term includes polyclonal and monoclonal antibodies as well as antibody fragments.

Bind, Binds or Interacts With: As used herein, “bind,” “binds,” or “interacts with” refers to that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ moles/liter for that second molecule.

Conserved: As used herein, a “conserved” H. somni amino acid fragment or protein, including IbpA, IbpB and TbpA, is one that is present in a particular H. somni protein in at least x % of H. somni. The value of x may be 50% or more, e.g., 66%, 75%, 80%, 90%, 95% or even 100% (i.e., the amino acid is found in the protein in question in all H. somni). In order to determine whether an amino acid is “conserved” in a particular H. somni protein, it is necessary to compare that amino acid residue in the sequences of the protein in question from a plurality of different H. somni (a reference population). The reference population may include a number of different H. somni species or may include a single species. The reference population may include a number of different serogroups of a particular species or a single serogroup. A preferred reference population consists of the 5 most common H. somni strains.

Heterologous: As used herein, the term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene.

Epitope: As used herein, the term “epitope” means antigenic determinant, and may elicit a cellular and/or humoral response.

Origin of Replication: As used herein, an “origin of replication” is a polynucleotide sequence that initiates and regulates replication of polynucleotides, such as an expression vector. The origin of replication behaves as an autonomous unit of polynucleotide replication within a cell, capable of replication under its own control. An origin of replication may be needed for a vector to replicate in a particular host cell. With certain origins of replication, an expression vector can be reproduced at a high copy number in the presence of the appropriate proteins within the cell. Examples of origins are the autonomously replicating sequences, which are effective in yeast; and the viral T-antigen, effective in COS-7 cells.

Mutant: As used herein, a “mutant” sequence is defined as a DNA, RNA or amino acid sequence differing from but having homology with the native or disclosed sequence. Depending on the particular sequence, the degree of homology (sequence identity) between the native or disclosed sequence and the mutant sequence is preferably greater than 50% (e.g., 60%, 70%, 80%, 90%, 95%, 99% or more) which is calculated as described above.

Allelic variant: As used herein, an “allelic variant” of a nucleic acid molecule, or region, for which nucleic acid sequence is provided herein is a nucleic acid molecule, or region, that occurs at essentially the same locus in the genome of another or second isolate, and that, due to natural variation caused by, for example, mutation or recombination, has a similar but not identical nucleic acid sequence. A coding region allelic variant typically encodes a protein having similar activity to that of the protein encoded by the gene to which it is being compared. An allelic variant can also comprise an alteration in the 5′ or 3′ untranslated regions of the gene, such as in regulatory control regions.

Controlled-Release Component: As used herein, the term “controlled-release component” refers to an agent that facilitates the controlled-release of a compound including, but not limited to, polymers, polymer matrices, gels, permeable membranes, liposomes, microspheres, or the like, or any combination thereof. Methods for producing compounds in combination with controlled-release components are known to those of skill in the art.

Pharmaceutically Acceptable: As used herein, the term “pharmaceutically acceptable” means approved by a regulatory agency of the Federal or a state government or listed in the U.S. Pharmacopeia or other generally recognized pharmacopeia for use in animals.

Pharmaceutically Acceptable Carrier: As used herein, the term “pharmaceutically acceptable carrier” refers to a diluent, adjuvant, excipient, or vehicle with which a compound is administered. Such carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when a compound is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A compound, if desired, can also combine minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compounds in combination with carriers are known to those of skill in the art.

Pharmaceutically Acceptable Salt: As used herein, the term “pharmaceutically acceptable salt” includes those salts of a pharmaceutically acceptable compound formed with free amino groups such as those derived from hydrochloric, phosphoric, acetic, oxalic, tartaric acids, and those formed with free carboxyl groups such as those derived from sodium, potassium, ammonium, calcium, ferric hydroxides, isopropylamine, triethylamine, 2-ethylamino ethanol, histidine, and procaine. If the compound is basic, salts may be prepared from pharmaceutically acceptable non-toxic acids including inorganic and organic acids. Such acids include acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Particularly preferred are besylate, hydrobromic, hydrochloric, phosphoric and sulfuric acids. If the compound is acidic, salts may be prepared from pharmaceutically acceptable organic and inorganic bases. Suitable organic bases include, but are not limited to, lysine, N,N′-dibenzylethylenediamine, chloroprocaine, choline, diethanolamine, ethylenediamine, meglumine (N-methylglucamine) and procaine. Suitable inorganic bases include, but are not limited to, alkaline and earth-alkaline metals such as aluminum, calcium, lithium, magnesium, potassium, sodium and zinc. Methods for synthesizing such salts are known to those of skill in the art.

Pro-drug: As used herein, the term “pro-drug” refers to any compound which releases an active drug in vivo when such a compound is administered to an animal subject. Pro-drugs can be prepared, for example, by functional group modification of an active drug. The functional group may be cleaved in vivo to release the active drug compound. Pro-drugs include, for example, compounds in which a group that may be cleaved in vivo is attached to a hydroxy, amino or carboxyl group in the active drug. Examples of pro-drugs include, but are not limited to esters (e.g., acetate, methyl, ethyl, formate, and benzoate derivatives), carbamates, amides and ethers. Methods for synthesizing such pro-drugs are known to those of skill in the art.

Therapeutically Effective Amount: As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition. This can be an immune response activating amount.

Histophilus Somni Antigens, Compositions and Methods of Use

This invention provides Histophilus somni polypeptide sequences and nucleotide sequences encoding the polypeptide sequences. With these disclosed sequences, nucleic acid probe assays and expression cassettes and vectors can be produced. The expression vectors can be transformed into bacterial or host cells to produce proteins. The purified or isolated polypeptides (which may also be chemically synthesized) can be used to detect antibodies to detect H. somni proteins or to produce antibodies against H. somni proteins. Also, the host cells or extracts can be utilized for biological assays to isolate agonists or antagonists. In addition, with these sequences one can search to identify open reading frames and identify amino acid sequences. The proteins may also be used in immunogenic compositions, antigenic compositions and as vaccine compositions.

The practice of the present invention will employ, unless otherwise indicated, conventional techniques of molecular biology, microbiology, recombinant DNA, and immunology, which are within the skill of the art. Such techniques are explained fully in the literature, e.g., Sambrook Molecular Cloning: A Laboratory Manual, Second Edition (1989); DNA Cloning, Volumes I and II (D. N Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification: Principles and Practice, Second Edition (Springer-Verlag, N.Y.), and Handbook of Experimental Immunology, Volumes I-IV (D. M. Weir and C. C. Blackwell eds 1986). Standard abbreviations for nucleotides and amino acids are used in this specification.

The methods and compositions of the present invention are provided as a result of the discovery that both the HMW and p76 IgBPs of H. somni are encoded by one large ORF (ibpA) with many potential translational initiation sites. Alternatively, ibpA and ibpB may be transcribed together. The ibpA gene and the flanking ibpB gene are a two-partner secretion pathway for large virulence exoproteins and their transporter proteins in various Gram-negative bacteria recently described by Jacob-Dubuisson et al. (29). Several of these exoproteins are fibrillar structures such as FHA of B. pertussis (32) and HMW1 and 2 of nontypeable H. influenzae (45, 46) as well as the H. somni IgBPs (18). The FHA protein processed from its precursor FhaB protein is a filamentous structure on the surface of B. pertussis, much like the IgBP surface fibrils of H. somni (18). FHA has been reported to have repeats in its N-terminal region (32, 47) and a later study has shown that tandem 19-residue repeats identified in the FHA sequence may contribute to the formation of the filamentous structure of FHA (48, 49). Likewise, the 22-residue repeats of ibpA may contribute to the fibrillar structure which we reported previously (18). Another factor which may be important in the fibrillar structure of ibpA is the EQ rich domain predicted to be coiled-coil structure, since coiled-coil α-helices are known to be important in the structure of bacterial fibrillar proteins such as streptococcal M proteins (50). Analysis of the P. multocida genome demonstrated that two large ORFs, pfhB1 and pfhB2 (22), encoding large exoproteins were similar to B. pertussis FhaB and contained a homologous sequence to H. somni p76 IgBP. The present discovery provides that these two P. multocida predicted gene products, especially the PfhB2 protein, showed high similarity to IbpA protein in their entire sequences, suggesting that both the P. multocida and H. somni genes may be derived from a common ancestor gene or a horizontal gene transfer from other common bacteria or from each other. The C terminal portion of IbpA has has been previously shown to include two insertion sequence like segments which were direct repeats (21). Similar large repeats were found in PfhB1 and PfhB2 proteins (22).

Functions of IbpA were addressed initially by studying Ig Fc binding, since that was the first function identified for this protein (16, 17). Bovine IgG2 Fc binding appears to involve the IbpA EQ rich domain (aa 1116-1255) which has homology to S. pyogenes surface IgBPs (41, 51) as well as an IbpA sequence at aa 3,354-3,698 within the p76 IgBP protein sequence which had homology with the IgA binding β antigen of S. agalactiae (52). The recombinant GST-ibpA3 fragment (aa 972-1,515) and the recombinant p76 (18) strongly bind bovine anti-DNP IgG2. These regions of H. somni IbpA which are homologous to streptococcal surface IgBP antigens may play roles in inhibition of complement mediated killing of H. somni that has been associated with the presence of these IgBPs (21). The Fc binding of IgG2 to H. somni may prevent the appropriate configuration for C1_{q} binding and the activation of the complement cascade.

Several binding motifs found in IbpA protein may be involved in adhesion of H. somni to endothelial cells. H. somni infection is characterized by pneumonia and septicemia with localization in many tissues resulting in meningoencephalitis, abortion, myocarditis or arthritis. It is thought that the organisms invade across the alveoli to the capillaries and, after hematogenous spread, move back across the endothelium from the lumenal surface of the vessels to the target tissue. Vasculitis is characteristic of H. somni infection (2, 5, and 53). B. pertussis FHA is reported to promote invasion of respiratory epithelial cell through the interaction of its RGD sequence with host cell α5β1 integrin (54). Others have shown that the RGD motif and the heparin binding domain are involved in attachment to endothelial cells (55-59). Competitive inhibition studies show attachment to bovine endothelial cells by heparin or dextran sulfate but not RGDS peptide suggest that bovine pulmonary artery endothelial cell adherence could be mediated by the IbpA heparin binding domain. It must be borne in mind, however, that heparin binding domains of other H. somni surface proteins may have contributed (if such domains exist). Binding of B. pertussis via RGD sequence of FHA to the CR3 integrin on macrophage cell surface is reported to result in phagocytosis without generation of an oxidative burst and survival of the bacteria in phagocytes (60, 61). The TK-D motif of FHA also has been implicated in interaction of FHA with CR3 integrin (38) so this may also modify macrophage killing. Similarly, unopsonized H. somni bacteria are taken up by bovine macrophages but are not killed (12, 62). Therefore, several motifs of ibpA are involved in the vasculitis, thrombosis and inhibition of macrophage killing characteristic of H. somni infection (1, 2, 12, 53, 62)

The immunigenicity of IbpA was studied and is shown to be similar to B. pertussis FHA which provides a similar fibrillar structure and sequence to H. somni IgBPs and is also a component of some of the new acellular pertussis vaccines (31, 32, 47). The peptide expressed by pGST-ibpA3 bound IgG2 Fcs most strongly and was recognized by convalescent phase serum. Accordingly, the present invention provides that IbpA polypeptides and fragments elicit immune protection in calves against intrabronchial challenge with a high dose of H. somni. In addition, the present invention provides novel methods to increase immune response in animals, particularly bison, bovine and sheep, and related vaccine compositions.

Vaccines

Vaccines according to the invention may either be prophylactic (i.e., to prevent infection) or therapeutic (i.e., to treat disease after infection).

Such vaccines comprise immunizing antigen(s), immunogen(s), polypeptide(s), protein(s) or nucleic acid, usually in combination with “pharmaceutically acceptable carriers,” which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (see Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (2) saponin adjuvants, such as Stimulon™ (Cambridge Bioscience, Worcester, Mass.) may be used or particles generated therefrom such as ISCOMs (immunostimulating complexes); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12, etc.), interferons (e.g., gamma interferon), macrophage colony stimulating factor (M-CSF), tumor necrosis factor (TNF), etc.; (5) detoxified mutants of a bacterial ADP-ribosylating toxin such as a cholera toxin (CT), a pertussis toxin (PT), or an E. coli heat-labile toxin (LT), particularly Lt-K63, LT-R72, CT-S109, PT-K9/G129; and (6) other substances that act as immunostimulating agents to enhance the effectiveness of the composition.

As mentioned above, muramyl peptides include, but are not limited to, N-acetyl-muramyl-L-threonyl-Disoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-s-n-glycero-3-hydroxyphosphoryloxy)-ethylamine (MTP-PE), etc.

The vaccine compositions comprising immunogenic compositions (e.g., which may include the antigen, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles. Alternatively, vaccine compositions comprising immunogenic compositions may comprise an antigen, polypeptide, protein, protein fragment or nucleic acid in a pharmaceutically acceptable carrier.

More specifically, vaccines comprising immunogenic compositions comprise an immune response activating amount of the immunogenic polypeptides, as well as any other of the above-mentioned components, as needed. It is expected that the amount will fall in a relatively broad range that can be determined through routine trials.

Typically, the vaccine compositions or immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

The immunogenic compositions are conventionally administered parenterally, e.g., by injection, either subcutaneously, intramuscularly, or transdermally/transcutaneously. Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents.

As an alternative to protein-based vaccines, DNA vaccination may be employed (e.g., Robinson & Torres (1997) Seminars in Immunology 9:271-283; Donnelly et al. (1997) Annu Rev Immunol 15:617-648).

Gene Delivery Vehicles

Vehicles for delivery of constructs including a coding sequence of a therapeutic of the invention, to be delivered to the animal for expression in the animal, can be administered either locally or systemically. These constructs can utilize viral or non-viral vector approaches in in vivo or ex vivo modality. Expression of such coding sequence can be induced using endogenous animal or heterologous promoters. Expression of the coding sequence in vivo can be either constitutive or regulated.

The invention includes gene delivery vehicles capable of expressing the contemplated nucleic acid sequences. The gene delivery vehicle is preferably a viral vector and, more preferably, a retroviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector. See generally, Jolly (1994) Cancer Gene Therapy 1:51-64; Kimura (1994) Human Gene Therapy 5:845-852; Connelly (1995) Human Gene Therapy 6:185-193; and Kaplitt (1994) Nature Genetics 6:148-153.

Retroviral vectors are well known in the art and we contemplate that any retroviral vector is employable in the invention, including B, C and D type retroviruses, xenotropic retroviruses (for example, NZB-XI, NZB-X2 and NZB9-I (see O'Neill (1985) J. Virol. 53:160) polytropic retroviruses, e.g., MCF and MCF-MLV (see Kelly (1983) J. Virol. 45:291), spumaviruses and lentiviruses, See RNA Tumor Viruses, Second Edition, Cold Spring Harbor Laboratory, 1985.

Portions of the retroviral vector may be derived from different retroviruses. For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

These recombinant retroviral vectors may be used to generate transduction competent retroviral vector particles by introducing them into appropriate packaging cell lines. Retrovirus vectors can be constructed for site-specific integration into host cell DNA by incorporation of a chimeric integrase enzyme into the retroviral particle. It is preferable that the recombinant viral vector is a replication defective recombinant virus.

Packaging cell lines suitable for use with the above-described retrovirus vectors are well known in the art, are readily prepared, and can be used to create producer cell lines (also termed vector cell lines or “VCLs”) for the production of recombinant vector particles. Preferably, the packaging cell lines are made from parent cell lines which do not require inactivation.

Preferred retroviruses for the construction of retroviral vectors include Avian Leukosis Virus, Bovine Leukemia Virus, Murine Leukemia Virus, Mink-Cell Focus-Inducing Virus, Murine Sarcoma Virus, Reticuloendotheliosis Virus and Rous Sarcoma Virus. Particularly preferred Murine Leukemia Viruses include 4070A and 1504A (Hartley and Rowe (1976) J. Virol. 19:19-25), Abelson (ATCC No. VR-999), Friend (ATCC No. VR-245), Graffi, Gross (ATCC Nol VR-590), Kirsten, Harvey Sarcoma Virus and Rauscher (ATCC No. VR-998) and Moloney Murine Leukemia Virus (ATCC No. VR-190). Such retroviruses may be obtained from depositories or collections such as the American Type Culture Collection (“ATCC”) in Rockville, Md. or isolated from known sources using commonly available techniques.

Delivery of the compositions of this invention into cells is not limited to the above mentioned viral vectors. Other delivery methods and media may be employed such as, for example, nucleic acid expression vectors, polycationic condensed DNA linked or unlinked to killed adenovirus alone. Additional approaches are described in Philip (1994) Mol Cell Biol 14:2411-2418 and in Woffendin (1994) Proc Natl Acad Sci 91:1581-1585.

Particle-mediated gene transfer may be employed. Briefly, the sequence can be inserted into conventional vectors that contain conventional control sequences for high level expression, and then incubated with synthetic gene transfer molecules such as polymeric DNA-binding cations like polylysine, protamine, and albumin, linked to cell targeting ligands such as asialoorosomucoid, as described in Wu & Wu (1987) J. Biol. Chem. 262:4429-4432, insulin as described in Hucked (1990) Biochem Pharmacol 40:253-263, galactose as described in Plank (1992) Bioconjugate Chem 3:533-539, lactose or transferrin.

Naked DNA may also be employed. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes can act as gene delivery vehicles. On non-viral delivery, the nucleic acid sequences encoding a polypeptide can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al (1994) Proc. Natl. Acad. Sci. USA 91(24):11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152.

A polynucleotide composition can comprise therapeutically effective amount from about 0.001 mg/kg to 50 mg/kg, in certain aspects 0.01 mg/kg to about 10 mg/kg, and in other aspects 0.1 mg/kg to 1 mg/kg of the polynucleotide in the individual to which it is administered.

Delivery Methods

Once formulated, the polynucleotide compositions of the invention can be administered (1) directly to the subject; (2) delivered ex vivo, to cells derived from the subject; or (3) in vitro for expression of recombinant proteins. The subjects to be treated can be animals (i.e., mammals and birds).

Direct delivery of the compositions will generally be accomplished by injection, either subcutaneously, intraperitoneally, intravenously or intramuscularly or delivered to the interstitial space of a tissue. Other modes of administration include oral and pulmonary administration, suppositories, and transdermal applications, needles, and gene guns or hyposprays. Dosage treatment may be a single dose schedule or a multiple dose schedule.

Methods for the ex vivo delivery and reimplantation of transformed cells into a subject are known in the art. Examples of cells useful in ex vivo applications include, for example, stem cells, particularly hematopoietic, lymph cells, macrophages, dendritic cells, or tumor cells.

Generally, delivery of nucleic acids for both ex vivo and in vitro applications can be accomplished by the following procedures, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Polynucleotide and Polypeptide Pharmaceutical Compositions

In addition to the pharmaceutically acceptable carriers and salts described above, the following additional agents can be used with polynucleotide and/or polypeptide compositions.

A. Polypeptides

One example are polypeptides which include, without limitation: asioloorosomucoid (ASOR); transferrin; asialoglycoproteins; antibodies; antibody fragments; ferritin; interleukins; interferons, granulocyte, macrophage colony stimulating factor (GM-CSF), granulocyte colony stimulating factor (G-CSF), macrophage colony stimulating factor (M-CSF), stem cell factor and erythropoietin. Viral antigens, such as envelope proteins, can also be used. Also, proteins from other invasive organisms, such as the 17 amino acid peptide from the circumsporozoite protein of plasmodium falciparum known as RII.

B. Hormones and Vitamins

Other groups that can be included are, for example: hormones, steroids, androgens, estrogens, thyroid hormone, or vitamins, folic acid.

C. Polyalkylenes and Polysaccharides

Also, polyalkylene glycol can be included with the desired polynucleotides or polypeptides. In a preferred embodiment, the polyalkylene glycol is polyethlylene glycol. In addition, mono-, di-, or polysaccharides can be included. In a preferred embodiment of this aspect, the polysaccharide is dextran or DEAE-dextran. Also, chitosan and poly(lactide-co-glycolide).

D. Lipids and Liposomes

The desired polynucleotide or polypeptide can also be encapsulated in lipids or packaged in liposomes prior to delivery to the subject or to cells derived therefrom.

Lipid encapsulation is generally accomplished using liposomes which are able to stably bind or entrap and retain nucleic acid. The ratio of condensed polynucleotide to lipid preparation can vary but will generally be around 1:1 (mg DNA:micromoles lipid), or more of lipid. For a review of the use of liposomes as carriers for delivery of nucleic acids, see, Hug and Sleight (1991) Biochim. Biophys. Acta. 1097:1-17; Straubinger (1983) Meth. Enzymol. 101:512-527.

Liposomal preparations for use in the present invention include cationic (positively charged), anionic (negatively charged) and neutral preparations. Cationic liposomes have been shown to mediate intracellular delivery of plasmid DNA (Feigner (1987) Proc. Natl. Acad. Sci. USA 84:7413-7416); mRNA (Malone (1989) Proc. Natl. Acad. Sci. USA 86:6077-6081); and purified transcription factors (Debs (1990) J. Biol. Chem. 265:10189-10192), in functional form.

Cationic liposomes are readily available. For example, N(1-2,3-dioleyloxy)propyl)-N,N,N-triethylammonium (DOTMA) liposomes are available under the trademark Lipofectin, from GIBCO BRL, Grand Island, N.Y. (See, also, Feigner supra). Other commercially available liposomes include transfectace (DDAB/DOPE) and DOTAP/DOPE (Boerhinger). Other cationic liposomes can be prepared from readily available materials using techniques well known in the art.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphosphatidyl ethanolamine (DOPE), among others. These materials can also be mixed with the DOTMA and DOTAP starting materials in appropriate ratios. Methods for making liposomes using these materials are well known in the art.

The liposomes can comprise multilamellar vesicles (MLVs), small unilamellar vesicles (SUVs), or large unilamellar vesicles (LUVs). The various liposome-nucleic acid complexes are prepared using methods known in the art. See e.g., Straubinger (1983) Meth. Immunol. 101:512-527; Szoka (1978) Proc. Natl. Acad. Sci. USA 75:4194-4198; Papahadjopoulos (1975) Biochim. Biophys. Acta 394:483; Wilson (1979) Cell 17:77); Deamer & Bangham (1976) Biochim. Biophys. Acta 443:629; Ostro (1977) Biochem. Biophys. Res. Commun. 76:836; Fraley (1979) Proc. Natl. Acad. Sci. USA 76:3348); Enoch & Strittmatter (1979) Proc. Natl. Acad. Sci. USA 76:145; Fraley (1980) J. Biol. Chem. (1980) 255:10431; Szoka & Papahadjopoulos (1978) Proc. Natl. Acad. Sci. USA 75:145; and Schaefer-Ridder (1982) Science 215:166.

E. Lipoproteins

In addition, lipoproteins can be included with the polynucleotide or polypeptide to be delivered. Examples of lipoproteins to be utilized include: chylomicrons, HDL, IDL, LDL, and VLDL. Mutants, fragments, or fusions of these proteins can also be used. Also, modifications of naturally occurring lipoproteins can be used, such as acetylated LDL. These lipoproteins can target the delivery of polynucleotides to cells expressing lipoprotein receptors. Preferably, if lipoproteins are included with the polynucleotide to be delivered, no other targeting ligand is included in the composition.

Naturally occurring lipoproteins comprise a lipid and a protein portion. The protein portion are known as apoproteins. At the present, apoproteins A, B, C, D, and E have been isolated and identified. At least two of these contain several proteins, designated by Roman numerals, AI, AII, AIV; CI, CII, CIII.

A lipoprotein can comprise more than one apoprotein. For example, naturally occurring chylomicrons comprises of A, B, C, and E. Over time these lipoproteins lose A and acquire C and E apoproteins. VLDL comprises A, B, C, and E apoproteins, LDL comprises apoprotein B; and HDL comprises apoproteins A, C, and E. The amino acid of these apoproteins are known and are described in, for example, Breslow (1985) Annu Rev. Biochem 54:699; Law (1986) Adv. Exp Med. Biol. 151:162; Chen (1986) J. Biol. Chem. 261:12918; Kane (1980) Proc. Natl. Acad. Sci. USA 77:2465; and Utermann (1984) Hum. Genet. 65:232.

Lipoproteins contain a variety of lipids including, triglycerides, cholesterol (free and esters), and phospholipids. The composition of the lipids varies in naturally occurring lipoproteins. For example, chylomicrons comprise mainly triglycerides. A more detailed description of the lipid content of naturally occurring lipoproteins can be found, for example, in Meth. Enzymol. 128 (1986). The composition of the lipids are chosen to aid in conformation of the apoprotein for receptor binding activity. The composition of lipids can also be chosen to facilitate hydrophobic interaction and association with the polynucleotide binding molecule.

Naturally occurring lipoproteins can be isolated from serum by ultracentrifugation, for instance. Such methods are described in Meth. Enzymol. (supra); Pitas (1980) J. Biochem. 255:5454-5460 and Mahey (1979) J. Clin. Invest. 64:743-750.

Lipoproteins can also be produced by in vitro or recombinant methods by expression of the apoprotein genes in a desired host cell. See, for example, Atkinson (1986) Annu. Rev. Biophys. Chem. 15:403 and Radding (1958) Biochim. Biophys. Acta. 30:443.

Lipoproteins can also be purchased from commercial suppliers, such as Biomedical Technologies, Inc., Stoughton, Mass., USA.

Further description of lipoproteins can be found in Zuckermann et al. PCT Appln. No. US/9714465.

F. Polycationic Agents

Polycationic agents can be included, with or without lipoprotein, in a composition with the desired polynucleotide or polypeptide to be delivered.

Polycationic agents, typically, exhibit a net positive charge at physiological relevant pH and are capable of neutralizing the electrical charge of nucleic acids to facilitate delivery to a desired location. These agents have both in vitro, ex vivo, and in vivo applications. Polycationic agents can be used to deliver nucleic acids to a living subject either intramuscularly, subcutaneously, etc.

The following are examples of useful polypeptides as polycationic agents: polylysine, polyarginine, polyornithine, and protamine. Other examples include histones, protamines, serum albumin, DNA binding proteins, non-histone chromosomal proteins, coat proteins from DNA viruses, such as X174, transcriptional factors also contain domains that bind DNA and therefore may be useful as nucleic aid condensing agents. Briefly, transcriptional factors such as C/CEBP, c-jun, c-fos, AP-1, AP-2, AP-3, CPF, Prot-1, Sp-1, Oct-1, Oct-2, CREP, and TFIID contain basic domains that bind DNA sequences.

Organic polycationic agents include: spermine, spermidine, and putrescence.

The dimensions and of the physical properties of a polycationic agent can be extrapolated from the list above, to construct other polypeptide polycationic agents or to produce synthetic polycationic agents.

Synthetic Polycationic Agents

Synthetic polycationic agents which are useful include, for example, DEAE-dextran, polybrene, Lipofect, and lipofectAMINE, are monomers that form polycationic complexes when combined with polynucleotides or polypeptides.

Immunodiagnostic Assays

H. somni antigens of the invention can be used in immunoassays to detect antibody levels (or, conversely, anti-H. somni antibodies can be used to detect antigen levels). Antibodies to H. somni proteins within biological samples, including for example, blood or serum samples, can be detected. Design of the immunoassays is subject to a great deal of variation, and a variety of these are known in the art. Protocols for the immunoassay may be based, for example, upon competition, or direct reaction, or sandwich type assays. Protocols may also, for example, use solid supports, or may be by immunoprecipitation. Most assays involve the use of labeled antibody or polypeptide; the labels may be, for example, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

Kits suitable for immunodiagnosis and containing the appropriate labeled reagents are constructed by packaging the appropriate materials, including the compositions of the invention, in suitable containers, along with the remaining reagents and materials (for example, suitable buffers, salt solutions, etc.) required for the conduct of the assay, as well as suitable set of assay instructions.

Nucleic Acid Hybridization

“Hybridization” refers to the association of two nucleic acid sequences to one another by hydrogen bonding. Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook et al.

“Stringency” refers to conditions in a hybridization reaction that favor association of very similar sequences over sequences that differ. For example, the combination of temperature and salt concentration should be chosen that is approximately 120 to 200° C. below the calculated Tm of the hybrid under study. The temperature and salt conditions can often be determined empirically in preliminary experiments in which samples of genomic DNA immobilized on filters are hybridized to the sequence of interest and then washed under conditions of different stringencies. See Sambrook et al. at page 9.50.

Variables to consider when performing, for example, a Southern blot are (1) the complexity of the DNA being blotted and (2) the homology between the probe and the sequences being detected. The total amount of the fragment(s) to be studied can vary from a magnitude of 10, from 0.1 to 1 μg for a plasmid or phage digest to 10⁻⁹ to 10⁻⁸ g for a single copy gene in a highly complex eukaryotic genome. For lower complexity polynucleotides, substantially shorter blotting, hybridization, and exposure times, a smaller amount of starting polynucleotides, and lower specific activity of probes can be used. For example, a single-copy yeast gene can be detected with an exposure time of only 1 hour starting with 1 μg of yeast DNA, blotting for two hours, and hybridizing for 4-8 hours with a probe of 10⁸ cpm/μg. For a single-copy animal gene a conservative approach would start with 10 μg of DNA, blot overnight, and hybridize overnight in the presence of 10% dextran sulfate using a probe of greater than 10⁸ cpm/μg, resulting in an exposure time of ˜24 hours.

Several factors can affect the melting temperature (Tₘ) of a DNA-DNA hybrid between the probe and the fragment of interest, and consequently, the appropriate conditions for hybridization and washing. In many cases the probe is not 100% homologous to the fragment. Other commonly encountered variables include the length and total G+C content of the hybridizing sequences and the ionic strength and formamide content of the hybridization buffer. The effects of all of these factors can be approximated by a single equation: Tₘ=81+16.6(log₁₀C₁)+0.4(%(G+C))−0.6(% formamide)−600/n−1.5(% mismatch) where Cᵢ is the salt concentration (monovalent ions) and n is the length of the hybrid in base pairs (slightly modified from Meinkoth & Wahl (1984) Anal. Biochem. 138: 267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (i.e., stringency), it becomes less likely for hybridization to occur between strands that are nonhomologous, and as a result, background decreases. If the radiolabeled probe is not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology between the probe and the target fragment are not known, the simplest approach is to start with both hybridization and wash conditions which are nonstringent. If non-specific bands or high background are observed after autoradiography, the filter can be washed at high stringency and reexposed. If the time required for exposure makes this approach impractical, several hybridization and/or washing stringencies should be tested in parallel.

Nucleic Acid Probe Assays

Methods such as PCR, branched DNA probe assays, or blotting techniques utilizing nucleic acid probes according to the invention can determine the presence of cDNA or mRNA. A probe is said to “hybridize” with a sequence of the invention if it can form a duplex or double-stranded complex, which is stable enough to be detected.

The nucleic acid probes will hybridize to the H. somni nucleotide sequences of the invention (including both sense and antisense strands). Though many different nucleotide sequences will encode the amino acid sequence, the native H. somni sequence is preferred because it is the actual sequence present in cells. mRNA represents a coding sequence and so a probe should be complementary to the coding sequence; single-stranded cDNA is complementary to mRNA, and so a cDNA probe should be complementary to the noncoding sequence.

The probe sequence need not be identical to the H. somni sequence (or its complement)—some variation in the sequence and length can lead to increased assay sensitivity if the nucleic acid probe can form a duplex with target nucleotides, which can be detected. Also, the nucleic acid probe can include additional nucleotides to stabilize the formed duplex. Additional H. somni sequence may also be helpful as a label to detect the formed duplex. For example, a non-complementary nucleotide sequence may be attached to the 5′ end of the probe, with the remainder of the probe sequence being complementary to a H. somni sequence. Alternatively, non-complementary bases or longer sequences can be interspersed into the probe, provided that the probe sequence has sufficient complementarity with the H. somni sequence in order to hybridize therewith and thereby form a duplex which can be detected.

The exact length and sequence of the probe will depend on the hybridization conditions, such as temperature, salt condition and the like. For example, for diagnostic applications, depending on the complexity of the analyte sequence, the nucleic acid probe typically contains at least 10-20 nucleotides, preferably 15-25, and more preferably at least 30 nucleotides, although it may be shorter than this. Short primers generally require cooler temperatures to form sufficiently stable hybrid complexes with the template.

Probes may be produced by synthetic procedures, such as the triester method of Matteucci et al. (J. Am. Chem. Soc. (1981) 103:3185), or according to Urdea et al. (Proc. Natl. Acad. Sci. USA (1983) 80:7461), or using commercially available automated oligonucleotide synthesizers.

The chemical nature of the probe can be selected according to preference. For certain applications, DNA or RNA are appropriate. For other applications, modifications may be incorporated, e.g., backbone modifications, such as phosphorothioates or methylphosphonates, can be used to increase in vivo half-life, alter RNA affinity, increase nuclease resistance, etc. (e.g., see Agrawal & Iyer (1995) Carr. Opin. Biotechnol. 6:12-19; Agrawal (1996) TIBTECH 14:376-387); analogues such as peptide nucleic acids may also be used (e.g., see Corey (1997) TIBTECH 15:224-229; Buchardt et al. (1993) TIBTECH 11:384-386).

Alternatively, the polymerase chain reaction (PCR) is another well-known means for detecting small amounts of target nucleic acids. Two “primer” nucleotides hybridize with the target nucleic acids and are used to prime the reaction. The primers can comprise sequence that does not hybridize to the sequence of the amplification target (or its complement) to aid with duplex stability or, for example, to incorporate a convenient restriction site. Typically, such sequence will flank the desired H. somni sequence.

A thermostable polymerase creates copies of target nucleic acids from the primers using the original target nucleic acids as a template. After a threshold amount of target nucleic acid are generated by the polymerase, they can be detected by more traditional methods, such as Southern blots. When using the Southern blot method, the labeled probe will hybridize to the H. somni sequence (or its complement).

Also, mRNA or cDNA can be detected by traditional blotting techniques described in Sambrook et al (supra). mRNA, or cDNA generated from mRNA using a polymerase enzyme, can be purified and separated using gel electrophoresis. The nucleic acids on the gel are then blotted onto a solid support, such as nitrocellulose. The solid support is exposed to a labeled probe and then washed to remove any unhybridized probe. Next, the duplexes containing the labeled probe are detected. Typically, the probe is labeled with a radioactive moiety.

Antibodies

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, altered antibodies, univalent antibodies, Fab proteins, and single domain antibodies.

Antibodies against the proteins of the invention are useful for affinity chromatography, immunoassays, and distinguishing/identifying H. somni proteins. Antibodies elicited against the proteins of the present invention bind to antigenic polypeptides or proteins or protein fragments that are present and specifically associated with strains of H. somni. In some instances, these antigens may be associated with specific strains, such as those antigens specific for the H. somni strains. The antibodies of the invention may be immobilized to a matrix and utilized in an immunoassay or on an affinity chromatography column, to enable the detection and/or separation of polypeptides, proteins or protein fragments or cells comprising such polypeptides, proteins or protein fragments. Alternatively, such polypeptides, proteins or protein fragments may be immobilized so as to detect antibodies bindably specific thereto.

These antibodies may be used to detect H. somni antigens in tissues of infected amimals for diagnisis.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antisera is obtained by bleeding the immunized animal into a glass or plastic container, incubating the blood at 25° C. for one hour, followed by incubating at 4° C. for 2-18 hours. The serum is recovered by centrifugation (e.g., 1000 g for 10 minutes). About 20-50 ml per bleed may be obtained from rabbits.

Monoclonal antibodies are prepared using the standard method of Kohler & Milstein (Nature(1975) 256:495-96), or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen, B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected MAb-secreting hybridomas are then cultured either in vitro (e.g., in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly ³²P and ¹²⁵I), electron-dense reagents, enzymes, and ligands having specific binding partners. Enzymes are typically detected by their activity. For example, horseradish peroxidase is usually detected by its ability to convert 3,3′,5,5′-tetramethylbenzidine TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with ¹²⁵I, or with an anti-biotin MAb labeled with HRP. Other permutations and possibilities will be readily apparent to those of ordinary skill in the art, and are considered as equivalents within the scope of the invention.

Antigens, immunogens, polypeptides, proteins or protein fragments of the present invention elicit formation of specific binding partner antibodies. These antigens, immunogens, polypeptides, proteins or protein fragments of the present invention comprise immunogenic compositions of the present invention. Such immunogenic compositions may further comprise or include adjuvants, carriers, or other compositions that promote or enhance or stabilize the antigens, polypeptides, proteins or protein fragments of the present invention. Such adjuvants and carriers will be readily apparent to those of ordinary skill in the art.

Antibodies of the invention can be used to assay the IbpA protein in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting ibpA protein expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (35S), tritium (³H), indium (¹¹²1n), and technetium (⁹⁹Tc); luminescent labels, such as luminol; and fluorescent labels, such as fluorescein and rhodamine, and biotin.

Pharmaceutical Compositions

Pharmaceutical compositions can comprise (include) either polypeptides, antibodies, or nucleic acid of the invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician.

For purposes of the present invention, a theraptueically effective dosage can be from about 0.001 mg/kg to 50 mg/kg, in certain aspects 0.01 mg/kg to about 10 mg/kg, and in other aspects 0.1 mg/kg to 1 mg/kg of the polypeptide in the individual animal to which it is administered. In certain aspects, the therapeutically effective dosage can be 0.2 mg/kg of the polypeptide in the individual animal to which it is administered. For purposes of the present invention, a therapeutically effective dosage can be from about 0.01 mg/kg to 50 mg/kg or 0.05 mg/kg to about 10 mg/kg of the polynucleotides in the individual animal to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the compositions of the invention can be administered directly to the subject. The subjects to be treated can be animals.

Direct delivery of the compositions will generally be accomplished by injection, either subcutaneously, intraperitoneally, intravenously or intramuscularly or delivered to the interstitial space of a tissue. Other modes of administration include oral and pulmonary administration, suppositories, and transdermal or transcutaneous applications, needles, and gene guns or hyposprays. Dosage treatment may be a single dose schedule or a multiple dose schedule.

Expression Systems

The H. somni nucleotide sequences can be expressed in a variety of different expression systems; for example, those used with mammalian cells, plant cells, baculoviruses, bacteria, and yeast.

i. Mammalian Systems

Mammalian expression systems are known in the art. A mammalian promoter is any DNA sequence capable of binding mammalian RNA polymerase and initiating the downstream (3′) transcription of a coding sequence (e.g., structural gene) into mRNA. A promoter will have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation (Sambrook et al. (1989) “Expression of Cloned Genes in Mammalian Cells” In Molecular Cloning: A Laboratory Manual, 2nd ed.).

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallothionein gene, also provide useful promoter sequences. Expression may be either constitutive or regulated (inducible). Depending on the promoter selected, many promoters may be inducible using known substrates, such as the use of the mouse mammary tumor virus (MMTV) promoter with the glucocorticoid responsive element (GRE) that is induced by glucocorticoid in hormone-responsive transformed cells.

The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter (Maniatis et al. (1987) Science 236:1237; Alberts et al. (1989) Molecular Biology of the Cell, 2nd ed.). Enhancer elements derived from viruses may be particularly useful, because they usually have a broader host range. Examples include the SV40 early gene enhancer (Dijkema et al (1985) EMBO J. 4:761) and the enhancer/promoters derived from the long terminal repeat (LTR) of the Rous Sarcoma Virus (Gorman et al. (1982b) Proc. Natl. Acad. Sci. 79: 6777) and from human cytomegalovirus (Boshart et al. (1985) Cell 41: 521). Additionally, some enhancers are regulatable and become active only in the presence of an inducer, such as a hormone or metal ion (Sassone-Corsi and Borelli (1986) Trends Genet. 2: 215; Maniatis et al. (1987) Science 236: 1237).

A DNA molecule may be expressed intracellularly in animal cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide.

Alternatively, foreign proteins can also be secreted from the cell into the growth media by creating chimeric DNA molecules that encode a fusion protein comprised of a leader sequence fragment that provides for secretion of the foreign protein in animal cells. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus tripartite leader is an example of a leader sequence that provides for secretion of a foreign protein in animal cells.

Usually, transcription termination and polyadenylation sequences recognized by animal cells are regulatory regions located 3′ to the translation stop codon and thus, together with the promoter elements, flank the coding sequence. The 3′ terminus of the mature mRNA is formed by site-specific post-transcriptional cleavage and polyadenylation (Birnstiel et al. (1985) Cell 41: 349; Proudfoot and Whitelaw (1988) ‘Termination and 3′ end processing of eukaryotic RNA. In Transcription and splicing (ed. B. D. Hames and D. M. Glover); Proudfoot (1989) Trends Biochem. Sci. 14: 105). These sequences direct the transcription of an mRNA which can be translated into the polypeptide encoded by the DNA. Examples of transcription terminator/polyadenylation signals include those derived from SV40 (Sambrook et al (1989) “Expression of cloned genes in cultured mammalian cells.” In Molecular Cloning: A Laboratory Manual).

Usually, the above described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (e.g., plasmids) capable of stable maintenance in a host, such as animal cells or bacteria. Mammalian replication systems include those derived from animal viruses, which require trans-acting factors to replicate. For example, plasmids containing the replication systems of papovaviruses, such as SV40 (Gluzman (1981) Cell 23:175) or polyomavirus, replicate to extremely high copy number in the presence of the appropriate viral T antigen. Additional examples of mammalian replicons include those derived from bovine papillomavirus and Epstein-Barr virus. Additionally, the replicon may have two replication systems, thus allowing it to be maintained, for example, in mammalian cells for expression and in a prokaryotic host for cloning and amplification. Examples of such mammalian-bacteria shuttle vectors include pMT2 (Kaufman et al. (1989) Mol. Cell. Biol. 9:946) and pHEBO (Shimizu et al. (1986) Mol. Cell. Biol. 6:1074).

The transformation procedure used depends upon the host to be transformed. Methods for introduction of heterologous polynucleotides into mammalian cells are known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are known in the art and include many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (e.g., Hep G2), and a number of other cell lines.

ii. Plant Cellular Expression Systems

There are many plant cell culture and whole plant genetic expression systems known in the art. Additional examples of genetic expression in plant cell culture has been described by Zenk, Phytochemistry 30: 38613863 (1991). Descriptions of plant protein signal peptides may be found in addition to the references described above in Vaulcombe et al., Mol. Gen. Genet. 209:33-40 (1987); Chandler et al., Plant Molecular Biology 3:407-418 (1984); Rogers, J. Biol. Chem. 260:3731-3738 (1985); Rothstein et. al., Gene 55:353-356 (1987); Whittier et al., Nucleic Acids Research 15:2515-2535 (1987); Wirsel et al., Molecular Microbiology 3:3-14 (1989); Yu et al., Gene 122:247-253 (1992). A description of the regulation of plant gene expression by the phytohormone, gibberellic acid and secreted enzymes induced by gibberellic acid can be found in R. L. Jones and J. MacMillin, Gibberellins: in: Advanced Plant Physiology, Malcolm B. Wilkins, ed., 1984 Pitman Publishing Limited, London, pp. 21-52. References that describe other metabolically-regulated genes: Sheen, Plant Cell, 2:1027-1038 (1990); Maas et al., EMBO J. 9:3447-3452 (1990); Benkel and Hickey, Proc. Natl. Acad. Sci. 84:1337-1339 (1987).

Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host. The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilmink and Dons, 1993, Plant Mol Biol Reptr, 11(2):165-185.

Sequences suitable for permitting integration of the heterologous sequence into the plant genome are also recommended. These might include transposon sequences and the like for homologous recombination as well as Ti sequences which permit random insertion of a heterologous expression cassette into a plant genome. Suitable prokaryote selectable markers include resistance toward antibiotics such as ampicillin or tetracycline. Other DNA sequences encoding additional functions may also be present in the vector, as is known in the art.

The nucleic acid molecules of the subject invention may be included into an expression cassette for expression of the protein(s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector.

A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested. Typically secretion in seeds are across the aleurone or scutellar epithelium layer into the endosperm of the seed. While it is not required that the protein be secreted from the cells in which the protein is produced, this facilitates the isolation and purification of the recombinant protein.

Since the ultimate expression of the desired gene product will be in a eucaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code, Reed and Maniatis, Cell 41:95-105, 1985.

The vector can be microinjected directly into plant cells by use of micropipettes to mechanically transfer the recombinant DNA. Crossway, Mol. Gen. Genet, 202:179-185, 1985. The genetic material may also be transferred into the plant cell by using polyethylene glycol, Krens, et al., Nature, 296, 72-74, 1982. Another method of introduction of nucleic acid segments is high velocity ballistic penetration by small particles with the nucleic acid either within the matrix of small beads or particles, or on the surface, Klein, et al., Nature, 327, 70-73, 1987 and Knudsen and Muller, 1991, Planta, 185:330-336 teaching particle bombardment of barley endosperm to create transgenic barley. Yet another method of introduction would be fusion of protoplasts with other entities, either minicells, cells, lysosomes or other fusible lipid-surfaced bodies, Fraley, et al., Proc. Natl. Acad. Sci. USA, 79, 1859-1863, 1982.

The vector may also be introduced into the plant cells by electroporation. (Fromm et al., Proc. Natl. Acad. Sci. USA 82:5824, 1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersion, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

iii. Baculovirus Systems

The polynucleotide encoding the protein can also be inserted into a suitable insect expression vector, and is operably linked to the control elements within that vector. Vector construction employs techniques which are known in the art. Generally, the components of the expression system include a transfer vector, usually a bacterial plasmid, which contains both a fragment of the baculovirus genome, and a convenient restriction site for insertion of the heterologous gene or genes to be expressed; a wild-type baculovirus with a sequence homologous to the baculovirus-specific fragment in the transfer vector (this allows for the homologous recombination of the heterologous gene in to the baculovirus genome); and appropriate insect host cells and growth media.

After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and methods for baculovirus/insect cell expression systems are commercially available in kit form from, inter alia, Invitrogen, San Diego Calif. (“MaxBac” kit). These techniques are generally known to those skilled in the art and fully described in Summers and Smith, Texas Agricultural Experiment Station Bulletin No. 1555 (1987) (hereinafter “Summers and Smith”).

Prior to inserting the DNA sequence encoding the protein into the baculovirus genome, the above described components, comprising a promoter, leader (if desired), coding sequence of interest, and transcription termination sequence, are usually assembled into an intermediate transplacement construct (transfer vector). This construct may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extrachromosomal element (e.g., plasmids) capable of stable maintenance in a host, such as a bacterium. The replicon will have a replication system, thus allowing it to be maintained in a suitable host for cloning and amplification.

Currently, the most commonly used transfer vector for introducing foreign genes into AcNPV is pAc373. Many other vectors, known to those of skill in the art, have also been designed. These include, for example, pVL985 (which alters the polyhedrin start codon from ATG to ATT, and which introduces a BamHI cloning site 32 basepairs downstream from the ATT; see Luckow and Summers, Virology (1989) 17:31.

The plasmid usually also contains the polyhedrin polyadenylation signal (Miller et al. (1988) Ann. Rev. Microbiol., 42:177) and a prokaryotic ampicillin-resistance (amp) gene and origin of replication for selection and propagation in E. coli.

Baculovirus transfer vectors usually contain a baculovirus promoter. A baculovirus promoter is any DNA sequence capable of binding a baculovirus RNA polymerase and initiating the downstream (5′ to 3′) transcription of a coding sequence (e.g., structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein, Friesen et al., (1986) “The Regulation of Baculovirus Gene Expression,” in: The Molecular Biology of Baculoviruses (ed. Walter Doerfler); and the gene encoding the p10 protein, Vlak et al., (1988), J. Gen. Virol. 69:765.

DNA encoding suitable signal sequences can be derived from genes for secreted insect or baculovirus proteins, such as the baculovirus polyhedrin gene (Carbonell et al. (1988) Gene, 73: 409). Alternatively, since the signals for animal cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin can also be used to provide for secretion in insects.

A recombinant polypeptide or polyprotein may be expressed intracellularly or, if it is expressed with the proper regulatory sequences, it can be secreted. Good intracellular expression of nonfused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is co-transformed with the heterologous DNA of the transfer vector and the genomic DNA of wild type baculovirus—usually by cotransfection. The promoter and transcription termination sequence of the construct will usually comprise a 2-5 kb section of the baculovirus genome. Methods for introducing heterologous DNA into the desired site in the baculovirus virus are known in the art. (See Summers and Smith supra; Ju et al. (1987); Smith et al., Mol. Cell. Biol. (1983) 3:2156; and Luckow and Summers (1989)). For example, the insertion can be into a gene such as the polyhedrin gene, by homologous double crossover recombination; insertion can also be into a restriction enzyme site engineered into the desired baculovirus gene. Miller et al., (1989), Bioessays 4:91. The DNA sequence, when cloned in place of the polyhedrin gene in the expression vector, is flanked both 5′ and 3′ by polyhedrin-specific sequences and is positioned downstream of the polyhedrin promoter.

The newly formed baculovirus expression vector is subsequently packaged into an infectious recombinant baculovirus. Homologous recombination occurs at low frequency (between about 1% and about 5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 μM in size, are highly refractile, giving them a bright shiny appearance that is readily visualized under the light microscope. Cells infected with recombinant viruses lack occlusion bodies. To distinguish recombinant virus from wild-type virus, the transfection supernatant is plaqued onto a monolayer of insect cells by techniques known to those skilled in the art. Namely, the plaques are screened under the light microscope for the presence (indicative of wild-type virus) or absence (indicative of recombinant virus) of occlusion bodies. Current Protocols in Microbiology Vol. 2 (Ausubel et al. eds) at 16.8 (Supp. 10, 1990); Summers and Smith, supra; Miller et al. (1989).

Recombinant baculovirus expression vectors have been developed for infection into several insect cells. For example, recombinant baculoviruses have been developed for, inter alia: Aedes aegypti, Autographa californica, Bombyx mori, Drosophila melanogaster, Spodoptera frugiperda, and Trichoplusia ni (Carbonell et al., (1985) J. Virol. 56:153; Wright (1986) Nature 321:718; Smith et al., (1983) Mol. Cell. Biol. 3:2156; and see generally, Fraser, et al. (1989) In Vitro Cell. Dev. Biol. 25:225).

Cells and cell culture media are commercially available for both direct and fusion expression of heterologous polypeptides in a baculovirus/expression system; cell culture technology is generally known to those skilled in the art. See, e.g., Summers and Smith supra.

The modified insect cells may then be grown in an appropriate nutrient medium, which allows for stable maintenance of the plasmid(s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, e.g., HPLC, affinity chromatography, ion exchange chromatography, etc.; electrophoresis; density gradient centrifugation; solvent extraction, or the like. As appropriate, the product may be further purified, as required, so as to remove substantially any insect proteins which are also secreted in the medium or result from lysis of insect cells, so as to provide a product which is at least substantially free of host debris, e.g., proteins, lipids and polysaccharides.

In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the art, based upon what is known in the art.

iv. Bacterial Systems

Bacterial expression techniques are known in the art. A bacterial promoter is any DNA sequence capable of binding bacterial RNA polymerase and initiating the downstream (3′) transcription of a coding sequence (e.g., structural gene) into mRNA. A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) (Raibaud et al. (1984) Annu. Rev. Genet. 18:173). Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) (Chang et al. (1977) Nature 198:1056), and maltose. Additional examples include promoter sequences derived from biosynthetic enzymes such as tryptophan (trp) (Goeddel et al. (1980) Nuc. Acids Res. 8:4057; Yelverton et al. (1981) Nucl. Acids Res. 9:731). The betalactamase (bla) promoter system (Weissmann (1981) “The cloning of interferon and other mistakes.” In Interferon 3 (ed. I. Gresser)), bacteriophage lambda PL (Shimatake et al. (1981) Nature 292:128) and T5 promoter systems also provide useful promoter sequences.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter. For example, the tac promoter is a hybrid trp-lac promoter comprised of both trp promoter and lac operon sequences that is regulated by the lac repressor (Amann et al. (1983) Gene 25:167; de Boer et al. (1983) Proc. Natl. Acad. Sci. 80:21). Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system (Studier et al. (1986) J. Mol. Biol. 189:113; Tabor et al. (1985) Proc Natl. Acad. Sci. 82: 1074). In addition, a hybrid promoter can also be comprised of a bacteriophage promoter and an E. coli operator region.

In addition to a functioning promoter sequence, an efficient ribosome binding site is also useful for the expression of foreign genes in prokaryotes. In E. coli, the ribosome binding site is called the Shine-Dalgarno (SD) sequence and includes an initiation codon (ATG) and a sequence 3-9 nucleotides in length located 3-11 nucleotides upstream of the initiation codon (Shine et al. (1975) Nature 254: 34). The SD sequence is thought to promote binding of mRNA to the ribosome by the pairing of bases between the SD sequence and the 3′ end of E. coli 16S rRNA (Steitz et al. (1979) “Genetic signals and nucleotide sequences in messenger RNA.” In Biological Regulation and Development: Gene Expression (ed. R. F. Goldberger)). To express eukaryotic genes and prokaryotic genes with weak ribosome-binding site, it is often necessary to optimize the distance between the SD sequence and the ATG of the eukaryotic gene (Sambrook et al. (1989) “Expression of cloned genes in Escherichia coli.” In Molecular Cloning: A Laboratory Manual).

A DNA molecule may be expressed intracellularly. A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo or in vitro incubation with a bacterial methionine N-terminal peptidase.

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example, the bacteriophage lambda cell gene can be linked at the 5′ terminus of a foreign gene and expressed in bacteria. The resulting fusion protein preferably retains a site for a processing enzyme (factor Xa) to cleave the bacteriophage protein from the foreign gene (Nagai et al. (1984) Nature 309:810). Fusion proteins can also be made with sequences from the lacZ (Jia et al. (1987) Gene 60:197), trpE (Allen et al. (1987) J Biotechnol. 5:93; Makoff et al. (1989) J. Gen. Microbiol. 135:11), and Chey genes. The DNA sequence at the junction of the two amino acid sequences may or may not encode a cleavable site. Another example is a ubiquitin fusion protein. Such a fusion protein is made with the ubiquitin region that preferably retains a site for a processing enzyme (e.g., ubiquitin specific processing-protease) to cleave the ubiquitin from the foreign protein. Through this method, native foreign protein can be isolated (Miller et al. (1989) Bio/Technology 7:698).

Alternatively, foreign proteins can also be secreted from the cell by creating chimeric DNA molecules that encode a fusion protein comprised of a signal peptide sequence fragment that provides for secretion of the foreign protein in bacteria. The signal sequence fragment usually encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) (Masui et al. (1983), in: Experimental Manipulation of Gene Expression; Ghrayeb et al. (1984) EMBO J. 3:2437) and the E. coli alkaline phosphatase signal sequence (phoA) (Oka et al. (1985) Proc. Natl. Acad. Sci. 82:7212). As an additional example, the signal sequence of the alpha-amylase gene from various Bacillus strains can be used to secrete heterologous proteins from B. subtilis (Palva et al. (1982) Proc. Natl. Acad. Sci. USA 79:5582).

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E. coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (e.g., plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recombinations between homologous DNA in the vector and the bacterial chromosome. For example, integrating vectors constructed with DNA from various Bacillus strains integrate into the Bacillus chromosome. Integrating vectors may be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline (Davies et al. (1978) Annu. Rev. Microbiol. 32:469). Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable marker that is either maintained in a replicon or developed into an integrating vector, as described above.

Expression and transformation vectors, either extra-chromosomal replicons or integrating vectors, have been developed for transformation into many bacteria. For example, expression vectors have been developed for, inter alia, the following bacteria: Bacillus subtilis (Palva et al. (1982) Proc. Natl. Acad. Sci. USA 79:5582), Escherichia coli (Shimatake et al. (1981) Nature 292:128; Amann et al. (1985) Gene 40:183; Studier et al. (1986) J. Mol. Biol. 189:113), Streptococcus cremoris (Powell et al. (1988) Appl. Environ. Microbiol. 54:655); Streptococcus lividans (Powell et al. (1988) Appl. Environ. Microbiol. 54:655), Streptomyces lividans.

Methods of introducing exogenous DNA into bacterial hosts are well-known in the art, and usually include either the transformation of bacteria treated with CaCl₂ or other agents, such as divalent cations and DMSO. DNA can also be introduced into bacterial cells by electroporation. Transformation procedures usually vary with the bacterial species to be transformed. (See, e.g., use of Bacillus: Masson et al. (1989) FEMS Microbiol. Lett. 60:273; Palva et al. (1982) Proc. Natl. Acad. Sci. USA 79:5582; use of Campylobacter: Miller et al. (1988) Proc. Natl. Acad. Sci. 85:856; and Wang et al. (1990) J. Bacteriol. 172:949; use of Escherichia coli: Cohen et al. (1973) Proc. Natl. Acad. Sci. 69:2110; Dower et al. (1988) Nucleic Acids Res. 16:6127; Kushner (1978) “An improved method for transformation of Escherichia coli with ColEI-derived plasmids. In Genetic Engineering: Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; use of Lactobacillus: Chassy et al. (1987) FEMS Microbiol. Lett. 44:173; use of Pseudomonas: Fiedler et al. (1988) Anal. Biochem 170:38; use of Staphylococcus: Augustin et al. (1990) FEMS Microbiol. Lett. 66:203; use of Streptococcus: Barany et al. (1980) J. Bacteriol. 144:698; Harlander (1987) “Transformation of Streptococcus lactis by electroporation, in: Streptococcal Genetics (ed. J. Ferretti and R. Curtiss II); Perry et al. (1981) Infect. Immun. 32:1295; Powell et al. (1988) Appl. Environ. Microbiol. 54:655; Somkuti et al. (1987) Proc. 4th Evr. Cong. Biotechnology 1:412.

v. Yeast Expression

Yeast expression systems are also known to one of ordinary skill in the art. A yeast promoter is any DNA sequence capable of binding yeast RNA polymerase and initiating the downstream (3′) transcription of a coding sequence (e.g., structural gene) into mRNA. A promoter will have a transcription initiation region which is usually placed proximal to the 5′ end of the coding sequence. This transcription initiation region usually includes an RNA polymerase binding site (the “TATA Box”) and a transcription initiation site. A yeast promoter may also have a second domain called an upstream activator sequence (UAS), which, if present, is usually distal to the structural gene. The UAS permits regulated (inducible) expression. Constitutive expression occurs in the absence of a UAS. Regulated expression may be either positive or negative, thereby either enhancing or reducing transcription.

Yeast is a fermenting organism with an active metabolic pathway, therefore sequences encoding enzymes in the metabolic pathway provide particularly useful promoter sequences. Examples include alcohol dehydrogenase (ADH), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK). The yeast PHO5 gene, encoding acid phosphatase, also provides useful promoter sequences (Myanohara et al. (1983) Proc. Natl. Acad. Sci. USA 80:1).

In addition, synthetic promoters which do not occur in nature also function as yeast promoters. For example, UAS sequences of one yeast promoter may be joined with the transcription activation region of another yeast promoter; creating a synthetic hybrid promoter. Examples of such hybrid promoters include the ADH regulatory sequence linked to the GAP transcription activation region. Other examples of hybrid promoters include promoters which consist of the regulatory sequences of either the ADH2, GAL4, GAL10, OR PHO5 genes, combined with the transcriptional activation region of a glycolytic enzyme gene such as GAP or PyK. Furthermore, a yeast promoter can include naturally occurring promoters of non-yeast origin that have the ability to bind yeast RNA polymerase and initiate transcription. Examples of such promoters include, inter alia, (Cohen et al. (1980) Proc. Natl. Acad. Sci. USA 77:1078; Henikoff et al. (1981) Nature 283:835; Hollenberg et al. (1981) Curr. Topics Microbiol. Immunol. 96:119; Hollenberg et al. (1979) “The Expression of Bacterial Antibiotic Resistance Genes in the Yeast Saccharomyces cerevisiae,” in: Plasmids of Medical, Environmental and Commercial Importance (eds. K. N. Timmis and A. Puhler); Mercerau-Puigalon et al. (1980) Gene 11:163; Panthier et al. (1980) Curr. Genet. 2:109).

A DNA molecule may be expressed intracellularly in yeast. A promoter sequence may be directly linked with the DNA molecule, in which case the first amino acid at the N-terminus of the recombinant protein will always be a methionine, which is encoded by the ATG start codon. If desired, methionine at the N-terminus may be cleaved from the protein by in vitro incubation with cyanogen bromide.

Fusion proteins provide an alternative for yeast expression systems, as well as in mammalian, plant, baculovirus, and bacterial expression systems. Usually, a DNA sequence encoding the N-terminal portion of an endogenous yeast protein, or other stable protein, is fused to the 5′ end of heterologous coding sequences. Upon expression, this construct will provide a fusion of the two amino acid sequences. For example, the yeast superoxide dismutase (SOD) gene, can be linked at the 5′ terminus of a foreign gene and expressed in yeast. The DNA sequence at the junction of the two amino acid sequences may or may not encode a cleavable site. Another example is a ubiquitin fusion protein. Such a fusion protein is made with the ubiquitin region that preferably retains a site for a processing enzyme (e.g., ubiquitin-specific processing protease) to cleave the ubiquitin from the foreign protein. Through this method, therefore, native foreign protein can be isolated.

Alternatively, foreign proteins can also be secreted from the cell into the growth media by creating chimeric DNA molecules that encode a fusion protein comprised of a leader sequence fragment that provide for secretion in yeast of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted yeast proteins, such as the yeast invertase gene and the A-factor gene. Alternatively, leaders of non-yeast origin, such as an interferon leader, exist that also provide for secretion in yeast.

Usually, transcription termination sequences recognized by yeast are regulatory regions located 3′ to the translation stop codon, and thus together with the promoter flank the coding sequence. These sequences direct the transcription of an mRNA which can be translated into the polypeptide encoded by the DNA. Examples of transcription terminator sequence and other yeast-recognized termination sequences, such as those coding for glycolytic enzymes.

Usually, the above described components, comprising a promoter, leader (if desired), coding sequence of interest, and transcription termination sequence, are put together into expression constructs. Expression constructs are often maintained in a replicon, such as an extrachromosomal element (e.g., plasmids) capable of stable maintenance in a host, such as yeast or bacteria. The replicon may have two replication systems, thus allowing it to be maintained, for example, in yeast for expression and in a prokaryotic host for cloning and amplification. Examples of such yeast-bacteria shuttle vectors include YEp24 (Botstein et al. (1979) Gene 8: 17-24), pCI/1 (Brake et al. (1984) Proc. Natl. Acad. Sci USA 81:4642-4646), and YRp17 (Stinchcomb et al. (1982) J. Mol. Biol. 158:157). In addition, a replicon may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Enter a high or low copy number vector may be selected, depending upon the effect of the vector and the foreign protein on the host. See, e.g., Brake et al., supra.

Alternatively, the expression constructs can be integrated into the yeast genome with an integrating vector. Integrating vectors usually contain at least one sequence homologous to a yeast chromosome that allows the vector to integrate, and preferably contain two homologous sequences flanking the expression construct. Integrations appear to result from recombinations between homologous DNA in the vector and the yeast chromosome (Orr-Weaver et al. (1983) Methods in Enzymol. 101: 228-245). An integrating vector may be directed to a specific locus in yeast by selecting the appropriate homologous sequence for inclusion in the vector. See Orr-Weaver et al., supra. One or more expression construct may integrate, possibly affecting levels of recombinant protein produced (Rine et al. (1983) Proc. Natl. Acad. Sci. USA 80:6750). The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of yeast strains that have been transformed. Selectable markers may include biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a suitable selectable marker may also provide yeast with the ability to grow in the presence of toxic compounds, such as metal. For example, the presence of CUP1 allows yeast to grow in the presence of copper ions (Butt et al. (1987) Microbiol, Rev. 51: 351).

Alternatively, some of the above described components can be put together into transformation vectors. Transformation vectors are usually comprised of a selectable marker that is either maintained in a replicon or developed into an integrating vector, as described above.

Expression and transformation vectors, either extrachromosomal replicons or integrating vectors, have been developed for transformation into many yeasts. For example, expression vectors and methods of introducing exogenous DNA into yeast hosts have been developed for, inter alia, the following yeasts: Candida albicans (Kurtz, et al. (1986) Mol. Cell. Biol. 6: 142); Candida maltosa (Kunze, et al. (1985) J. Basic Microbiol. 25: 141); Hansenula polymorpha (Gleeson, et al. (1986) J. Gen. Microbiol. 132:3459; Roggenkamp et al. (1986) Mol. Gen. Genet. 202: 302); Kluyveromyces fragilis (Das, et al. (1984) J. Bacteriol. 158:1165); Kluyveromyces lactis (De Louvencourt et al. (1983) J. Bacteriol. 154:737; Van den Berg et al. (1990) Bio/Technology 8:135); Pichia guillerimondii (Kunze et al. (1985) J. Basic Microbiol. 25: 141); Pichia pastoris (Cregg, et al. (1985) Mol. Cell. Biol. 5:3376); Saccharomyces cerevisiae (Hinnen et al. (1978) Proc. Natl. Acad. Sci. USA 75:1929; Ito et al. (1983) J. Bacteriol. 153:163); Schizosaccharomyces pombe (Beach and Nurse (1981) Nature 300:706); and Yarrowia lipolytica (Davidow, et al. (1985) Curr. Genet. 10: 380471 Gaillardin, et al. (1985) Curr. Genet. 10: 49).

Methods of introducing exogenous DNA into yeast hosts are well-known in the art, and usually include either the transformation of spheroplasts or of intact yeast cells treated with alkali cations. Transformation procedures usually vary with the yeast species to be transformed. See, e.g., (Kurtz et al. (1986) Mol. Cell. Biol. 6:142; Kunze et al. (1985) J. Basic Microbiol. 25: 141; Candida); (Gleeson et al. (1986) J. Gen. Microbiol. 132:3459; Roggenkamp et al. (1986) Mol. Gen. Genet. 202:302; Hansenula); (Das et al. (1984) J. Bacteriol. 158:1165; De Louvencourt et al. (1983) J. Bacteriol. 154:1165; Van den Berg et al. (1990) Bio/Technology 8: 135; Kluyveromyces); (Cregg et al. (1985) Mol. Cell. Biol. 5:3376; Kunze et al. (1985) J. Basic Microbiol. 25:141; Pichial; (Hinnen et al. (1978) Proc. Natl. Acad. Sci. USA 75;1929; Ito et al. (1983) J. Bacteriol. 153:163; Saccharomyces); Beach and Nurse (1981) Nature 300:706; Schizosaccharomyces); (Davidow et al. (1985) Curr. Genet. 10:39; Gaillardin et al. (1985) Curr. Genet. 10:49; Yarrowia).

### Examples

Aspects of the present teachings may be further understood in light of the following examples, which should not be construed as limiting the scope of the present teachings in any way.

### Example 1

### H. somni Genetic and Functional Studies

A. Bacterial Strains and Plasmids

The virulent H. somni IgBP-positive strain 2336 (from a pneumonic calf) and the commensal IgBP-negative strain 1 P (from an asymptomatic carrier) have been described previously (11, 13, and 19). H. somni was grown on brain heart infusion (BHI) agar (Difco Laboratories, Detroit, Mich.) supplemented with 5% bovine blood and 0.5% yeast extract (Difco Laboratories) or Columbia blood agar base (Difco Laboratories) supplemented with 5% sheep defibrinated blood in candle jars or under 7% CO₂ in a CO₂ incubator at 37° C. For broth culture, BHI broth (Difco Laboratories) supplemented with 0.1% Tris and 0.01% thiamine monophosphate (63) or Columbia broth supplemented similarly with Tris and thiamine monophosphate was used with shaking at 37° C. E. coli was grown in Luria-Bertani (LB) broth or on LB agar plates at 37° C.

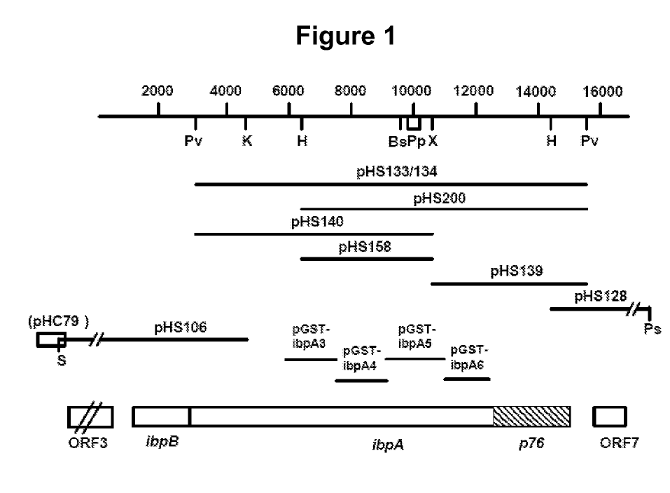

Plasmids used in this study (FIG. 1) included pHS133, pHS134, pHS140, pHS139 and pHS158 as previously described (19). The plasmid pHS158 contains the same insert as pHS155 (19), but in the opposite orientation. To construct pHS200, a 4.7 kb XbaI fragment was isolated from pHS139 and ligated to pHS158 digested with XbaI. The plasmid pHS132 was obtained by ligating a 3.8 kb BglII fragment into pUC19 in the previous subcloning study (19). To determine the sequence upstream from pHS140 and downstream of pHS139, plasmids pHS106 and pHS128 were constructed by ligating an approximately 8 kb KpnI-SalI fragment from the 5′ end and 4 kb HindIII-PstI central portion of pHS1 insert (20) into pUC18, respectively. All of the above plasmids were transformed into E. coli DH5α. An additional plasmid was constructed to read the sequence of the sense strand between BsilWI and XbaI sites by deleting the 5′ repetitive sequences, including six PpuMI sites, of the XbaI site. After electroporating pHS140 into E. coli G2929, the plasmid pHS140 without dcm methylation in the resultant transformant was digested with PpuMI, the approximately 3 kb DNA fragment was isolated, self-ligated and then transformed into E. coli DH5α. Standard recombinant DNA methods including restriction enzyme digestions, ligation reactions, and agarose gel electrophoresis were performed as described by Sambrook et al. (64).

B. DNA Sequencing

Both strands of the ibpA and ibpB loci were sequenced. To obtain the sequence of the antisense strand of the DNA insert in pHS158, a nested set of exonuclease III deletions were made using the Exonuclease III-Mung Bean Nuclease Deletion kit (Epicentre Technologies, Madison, Wis.) as suggested by the manufacturer. DNA sequencing was performed by the dideoxy method with Sequenase Version 2.0 DNA Sequencing kit (U.S. Biochemicals, Cleveland, Ohio) as suggested by the manufacturer. Sequencing of the sense strand of pHS158 DNA insert and the both strands of the 5′ region of pHS140 DNA insert was carried out at the DNA Sequencing Facility, Iowa State University, Ames, Iowa, with templates from pHS158, pHS140 and the 3 kb plasmid constructed from pHS140 by PpuMI digestion. Sequencing of a part of pHS1 and pHS134 DNA inserts was performed by using a model 377 automated DNA sequencer (Applied Biosystems Japan, Inc., Tokyo, Japan) to read a joining region between pHS158 and pHS139 DNA inserts and the 3′ region from XbaI site in the pHS134 DNA insert. Sequencing the 5′ and 3′ portion of the ibpA ORF was also performed similarly with templates from pHS1 as well as pHS106 and pHS128. Both strands of the DNA constituting the ibpA and ibpB loci were sequenced in their entirety.

C. Sequence Data Analysis

Sequence data were analyzed by DNASIS-Mac (Hitachi Software Engineering Co., Ltd., Tokyo, Japan) and Genetyx-Mac (Software Development Co. Ltd., Tokyo, Japan) as well as programs from various servers, including BESTFIT in GCG Sequence Analysis Software Package (36) and BLAST (28) (DNA Information and Stock Center, National Institute of Agrobiological Resources, Japan), SOSUI (23) (Tokyo University of Agriculture and Technology, Japan), COILS (40) (EMBnet, Switzerland) as well as SignalP (26) (Center for Biological Sequence Analysis, Denmark).

D. Expression and Purification of Fusion Proteins

The regions of 1,290 bp (nt 2,806-4,095, encoding aa 1-430 of ibpA), 1,623 bp (nt 4,096-5,718, aa 431-971), 1,632 bp (nt 5,719-7,350, aa 972-1,515 ), 1,665 bp (nt 7,351-9,015, aa 1,516-2,070), 1,983 bp (nt 9,013-10,995, aa 2,070-2,730), 1,413 bp (nt 10,996-12,408, aa 2,731-3,201) were amplified by PCR from pHS106 or pHS134 as template with primers

GSTP1F
(5′-CGGAATTCCGATGAATAAGAATTGTTATAAGCTTATTTTC-3′;
SEQ ID NO: 6),
GSTP1R
(5′-GCGTCGACTTACTCATTTGCTATAGTAATTCTTTTGGCGT-3′;
SEQ ID NO: 7),
GSTP2F
(5′-GCGGATCCATTACGGCGGATAAGTCTATCACAATAACC-3′;
SEQ ID NO: 8),
GSTP2R
(5′-GCGTCGACTTATTCAGAAAATCCTCCGGTTGAACCATTTTT-3′;
SEQ ID NO: 9),
GSTP3F
(5′-GCGCGGATCCAGTGAACGAATCACTGTAGGACAACATAAA-3′;
SEQ ID NO: 10),
GSTP3R
(5′-GCGCGTCGACTCAAGCTTTCACAAAACCTGTTTTACCTTT-3′;
SEQ ID NO: 11),
GSTP4F
(5′-GCGGATCCCGTAATTTTAATACGACAGATACTCACAGA-3′;
SEQ ID NO: 12),
GSTP4R
(5′-GCGTCGACTTATTTAATTTGTCCGGTAATTCTGTTCGCAAC-3′;
SEQ ID NO: 13),
GSTP5F
(5′-GCGGATCCAAAGATTTGGATCTTGTAGCGGCTCATTTC-3′;
SEQ ID NO: 14),
GSTP5R
(5′-GCGTCGACTTAGGCATAAATATGATCTGCCGTATCAACTTCCTGA
GT-3′; SEQ ID NO: 15),
GSTP6F
(5′-GCGGATCCGACATTAATGATGTTGTAAATAGAGCGAAT-3′;
SEQ ID NO: 16),
GSTP6R
(5′-GCGTCGACTTATTCTTCTTTCAATTTTGGATTACGTTGAAT-3′;
SEQ ID NO: 17),

respectively. The primers contained BamHI, EcoRI or SalI restriction enzyme sites (underlined in the primer sequences) to facilitate the insertion of the amplified product into the expression vector. The PCR products were cloned into vector pCRII using the TA Cloning kit (Invitrogen Corp., Carlsbad, Calif.) as suggested by the manufacturer. The cloned fragments were digested with both EcoRI and SalI or both BamHI and SalI, purified by using the QIAquick Gel Extraction kit (Qiagen K.K., Tokyo, Japan), and then ligated in frame to the gene encoding glutathione-S-transferase (GST) in vector pGEX-KG (65). E. coli DH5α transformants with each expression plasmid were screened for the expression of GST-fused protein by analysis on SDS-PAGE. The first two plasmid constructs that encoded N-terminal aa 1-430 or aa 431-971 were unstable in E. coli and failed to express GST-fused recombinant fragments. The last four plasmid constructs successfully expressed GST-fused recombinant fragments and were designated as pGST-ibpA3, pGST-ibpA4, pGST-ibpA5 and pGST-ibpA6. Selected clones, that expressed fusion proteins with appropriate size, were used for large scale expression. Forty milliliters of an overnight culture was inoculated into 450 ml of LB broth containing ampicillin at 100 μg/ml. The culture was incubated with shaking vigorously at 37° C. until an absorbance of 0.6 at 600 nm was reached. IPTG was then added at a final concentration of 0.5 mM and the culture was incubated for an additional 3 hours. Bacterial cells were harvested by centrifugation and resuspended in 50 ml of PBST (PBS containing 1% Triton X-100, aprotinin 20 μg/ml, leupeptin 5 μg/ml and 5 mM EDTA). Bacterial cells were sonicated and then the bacterial lysate was centrifuged at 10,000×g for 10 min at 4° C. The supernatant containing soluble GST-fusion protein was filtered through a 0.45 μm membrane filter, and then applied to a GST-Sepharose CL4B (Amersham Biotech, Tokyo, Japan) affinity column. The column was washed with PBST followed by Tris buffer [50 mM Tris-HCl (pH 8.0)-50 mM NaCl-1 mM EDTA-1 mM DTT] to remove unbound materials in the supernatant and then eluted with Tris buffer containing 10 mM glutathione (reduced type). The eluted fractions containing the GST-fused protein were pooled and concentrated by ultrafiltration (Centricon YM-30; Millipore, Bedford, Mass.).

E. Antibodies

Bovine IgG2 antibody to the hapten dinitrophenol (DNP) has been described previously (18). The IgG2 anti-DNP was absorbed sequentially with boiled extracts and pellets from H. somni 1P lacking IgBPs and with E. coli DH5α. Absorbed IgG2 anti-DNP contained approximately 100 μg of IgG2/ml. Convalescent phase serum and pre-infection serum from a calf (9001) with experimental H. somni induced pneumonia were described previously (42).

F. SDS-PAGE and Western Blotting

Whole bacterial cells and a concentrated culture supernatant were prepared as described previously (16, 43). In brief, recombinant E. coli clones, grown overnight in LB broth containing ampicillin at 100 μg/ml, were adjusted to an absorbance of 0.4 at 600 nm. Then 1.5 ml of this suspension was centrifuged at 13,000×g for 5 min to collect bacterial cell pellets. H. somni bacterial cells were harvested from agar plate cultures, rinsed in sterile PBS, and then resuspended in sterile distilled water for stock cell suspension. H. somni culture supernatant was separated from overnight broth culture of strain 2336 by centrifugation at 10,000×g for 10 min, filtered through a 0.45 μm filter, and dialyzed against 10 mM Tris-HCl, pH 7.5. The dialyzed supernatant was lyophilized and resuspended in 10 mM Tris-HCl, pH 7.5, to one-fiftieth the original volume. The bacterial cell pellets, the bacterial cell suspension and the concentrated culture supernatant were then stored at −70° C. until use.

Western blotting was done as described previously (11) with modifications. In brief, whole cells, concentrated culture supernatant and GST-fused ibpA fragments were mixed with 2× SDS sample buffer and solubilized at 100° C. for 5 min. Samples were then loaded onto 8% SDS-PAGE gels (prepared with standard acrylamide solution—acrylamide/bisacrylamide at 37.5:1) or 12% gels [prepared with acrylamide/bisacrylamide at 222:1 to enhance electrophoretic transfer (66)]. After electrophoresis, antigens were electrotransferred to nitrocellulose sheets in transfer buffer composed of 25 mM Tris, 192 mM glycine, 0.02% SDS, and 20% methanol as described previously (67). The blots of E. coli recombinants were reacted with absorbed IgG2 anti-DNP (100 μg/ml). The blots were reacted either with bovine anti-DNP IgG2 or with pre- or post infection serum from a calf (#9001) with experimental H. somni pneumonia as previously described (42). This was followed by a 1:1,000 dilution of peroxidase-conjugated goat anti-bovine IgG (Kirkegaard and Perry Laboratories, Gaithersburg, Md.). Color was developed with 4-chloro-1-naphthol plus hydrogen peroxide solution.

G. Inhibition of Adherence to Bovine Endothelial Cells

Bovine pulmonary artery endothelial cells were obtained from Drs C J Czuprynski and M J Sylte at the University of Wisconsin, Madison. Cell cultures and adherence assays were done as previously described (43). In brief, H. somni cells were incubated in the presence or absence of inhibitors with Formalin fixed endothelial cells in microtiter wells (Costar Corp., Cambridge Mass.) at an MOI of approximately 300:1. Inhibitors were added to wells [final concentrations of 4, 2, 1 and 0.5 mM concentrations of RGDS, RGES, heparin (sodium salt), dextran sulfate (approximate molecular weight of 10,000), dextran (approximate molecular weight of 9,500), all from Sigma-Aldrich (St. Louis, Mo.)] followed by addition of bacterial suspension of H. somni strain 2336 grown for 5.5 hours on BHITT plates. Plates were incubated for 1.5 hours at 37% and 6.6% CO₂. After washing extensively and fixing with 5% Formalin, the plate was blocked overnight with 3% gelatin in PBS with 0.02% azide and washed again 5 times. The plate was developed with monoclonal antibody 3G9 supernatant at a 1:10 dilution for 1 hour at 37° C. Monoclonal antibody 3G9 was previously shown to recognize H. somni 39K antigen and not to react with other bacteria (68). Plates were washed again, reacted with anti-mouse IgM, IgG and IgA horseradish peroxidase conjugate (Zymed Labs inc., San Francisco, Calif.) followed by TMB (Tetramethylbenzidine/hydrogen peroxide) substrate (KPL). Adherence of H. somni was determined by reading at A450/A650 in a dual wavelength microplate reader (Molecular Devices Corp., Menlo Park, Calif.).

H. Calf Immunization and Challenge

Six to eight-week-old calves were immunized with 500 μg of purified GST-fused recombinant protein or with 150 μg of purified GST recombinant protein in Freund's complete adjuvant subcutaneously at four sites and then boosted similarly with antigen in Freund's incomplete adjuvant two times at three-week-intervals. Two weeks after the second boosting, calves were sedated by injecting xylazine (0.1 mg/kg body weight) intramuscularly and then challenged intrabronchially with 10¹⁰ CFU of H. somni 2336 by using a fiberopticscope (Olympus, Tokyo, Japan). This is a very large challenge dose, since we previously showed that 10⁷ H. somni organisms cause pneumonia in 6-8 week old calves (53). Serum was collected for antibody analysis at 8 weeks after the first immunization, just before challenge. The following clinical signs were monitored twice daily following the challenge: temperature, heart rate, respiratory rate, cough, apathy, anorexia, dyspnea, nasal discharge, increased bronchial sounds as described by Primal et al. (69). Each sign was assigned a numerical value according to the following scale: for temperature, 0=<39.4° C., 1=39.5-40.0° C., 2=40.1-40.6° C., and 3=>40.6° C.; for heart rate, 0=60-90/min, 1=91-120/min, 2=121-150/min, and 3=>150/min; for respiratory rate, 0=36/min, 1 =37-60/min, 2=61-78/min, and 3=>79/min; and for all other subjective parameters, 0=normal, 1 =mild, 2=moderate and 3=marked severity of clinical signs. These numerical values were added together for each calf at each observation time. The mean of all values for all observation times equaled the mean clinical score. The necropsy for the calf that died was performed immediately. Euthanasia and necropsy for the remaining calves were done at 5 days after challenge. Input of image of consolidated pneumonic lung and whole lung at both dorsal and ventral surfaces was performed through scanning positive films photographed at necropsy. The pneumonic lesion and whole lung areas were measured by a computer analysis system (Ultimage v. 2.6; Solution Systems, Chiba, Japan). The ratio of total involved area to total lung area from both dorsal and ventral sides was determined. Tissue samples for bacteriological tests were obtained from several sites of affected region of the lung and homogenized samples were serially diluted and inoculated on blood agar plates to estimate CFU.

Results

Analysis of ibpA Expression and IgG2 Binding Function

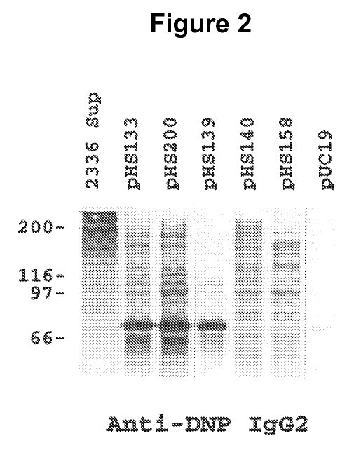

Subclones of ibpA (see FIG. 1) were expressed in E. coli and analyzed by Western blotting with anti-DNP IgG2 to determine which expressed fragments bound IgG2 by the Fc portion (i.e. were IgBPs) (FIG. 2).

In FIG. 1, restriction site abbreviations are: B, BamHI; Bs, BsiWI; H, HindIII; K, KpnI; Pp, PpuMI; Ps, PstI; Pv, PvuII; S, SalI; X, XbaI. A box at the Pp site was used to show six repetitive PpuMI sites at the position. A PvuII fragment from an H. somni cosmid clone was subcloned into HincII site in pUC19 (19), creating a new PpuMI site in the 5′ end of the insert. The SalI site was in the sequence of cosmid vector pHC79. The ibpA ORF began at a putative initiation ATG codon at nucleotide position (nt) of 2,806-2,808 and continuing for 12,285 bp (including the sequence labeled p76). The ibpB ORF started with a putative initiation ATG codon at nt 1,021-1,023 and ended at a TGA stop codon at 2,779-2,781. Nucleotide numbers are in accordance with the sequence including the ibpA and ibpB ORFs deposited in DDBJ (GenBank Accession No. AB087258).

In FIG. 2, antigens include H. somni concentrated culture supernatant from strain 2336 and whole bacterial lysates from recombinant E. coli clones with pHS133, pHS200, pHS140, pHS158, pHS139, and pUC19. Culture supernatant of strain 2336 (containing HMW-igBPs but not p76) and whole recombinant antigens were electrotransferred to a nitrocellulose membrane. The blot was reacted with bovine IgG2 anti-DNP at 100 μg/ml to assess Fc binding. Molecular mass standards (in kilodaltons) are noted on the left.

The anti-DNP IgG2 was used because we previously showed by lack of competitive inhibition by DNP-albumin or DNP (11, 68) that anti-DNP IgG2 reacts only via non-immune Fc binding to IgBPs Results showed that H. somni 2336 shed a series of HMW IgBPs into the culture supernatant (FIG. 2). The p76 IgBP was not detected in the supernatant as it is not shed, but remains on the cell surface as reported previously (11). Recombinant E. coli clones harboring pHS133 or pHS200 expressed both truncated HMW and the p76 IgBPs, but those harboring pHS139 expressed only the p76 IgBP and its truncated peptides. E. coli with pHS140 or pHS158, which contain the 7.7 kb DNA fragment or 4.2 kb DNA fragment upstream of pHS139 insertion sequence respectively, expressed only HMW IgBPs, while the clone harboring pHS 158 expressed only somewhat truncated HMW IgBPs (FIG. 2). Our efforts to construct an E. coli clone harboring plasmid with full ibpA ORF failed because insertion of full ibpA sequence to pUC18 produced only unstable clones with deleted insert of variable sizes. Although E. coli clones harboring pHS140, pHS200 or pHS133 all expressed HMW IgBPs above the 200 kDa marker, only the clone with pHS133 faintly expressed IgBPs of near sizes as high molecular mass as the H. somni 2336 culture supernatant. The gene expressing the full series of IgBPs is referred to as ibpA.

Nucleotide Sequence

Sequence analysis of the ibpA gene and its 5′- and 3′-flanking region, including the insert in pHS140 as well as upstream (pHS106) and downstream (pHS128) regions, identified a large ORF beginning at an ATG putative initiation codon [nucleotide (nt) 2,806-2,808] and continuing for 12,285 bp to the 3′-terminus of the p76 gene (nt 15,090) (FIG. 1). All nucleotide sequence numbers are based on the submitted nt sequence-accession number AB087258. The sequence analysis also revealed two additional ORFs within the 5′-flanking region of ibpA as well as an additional ORF at 3′-flanking region of ibpA. A proximal ORF, 5′ of ibpA was designated ibpB gene and starts with an ATG codon at nt 1,021-1,023 and ends with a TGA stop codon at nt 2,779-2,781. Upstream of the ibpB gene, a partial ORF (ORF3 in FIG. 1) was located on the opposite strand and was similar to a Pasteurella multocida ORF of unknown function (22). Within the ibpA 3′-flanking region an additional ORF (ORF7 in FIG. 1) was identified. The predicted protein encoded by this ORF had 64% identity to the P. multocida thiamin binding protein A (22). The putative ibpA initiation codon (nt 2,806-2,808) is located 24 bp from the termination codon of ibpB gene and is preceded by a purine-rich potential Shine-Dalgarno (SD) region (AGGAAG; nt 2,795-2,800). A putative −10 (TGGAGT; nt 2,711-2,716) and −35 (TTGACT; nt 2,689-2,694) promoter sequences for ibpA falls within the ibpB coding region. The ibpB gene was preceded by a putative SD region (AAGGA; nt 1,007-1,011) and potential promoter sequences, −10 (TATAAT; nt 983-988) and −35 (TTACCA; nt 957-962). Examination of the ibpA sequence revealed 18 additional putative ATG codons in the same frame and putative SD region and promoter sequences upstream from at least 12 of 18 putative ATG codons. Further analysis of the ibpA nucleotide sequence revealed that there were two sets of tandem repeats in addition to the two large direct repeats previously reported in the p76 gene (21). The first set of four (approximately 200-bp) repeating units begins at nt position 9,553 and ends with the last unit at nt 10,386. The similarities among three 210-bp units were over 95% but a fourth repeat of 204 bp was less similar at 58%. The two 285-bp repeating units begin at nt 10,432 and end with the second unit at nt 10,992. The similarity between these two 285-bp units was 73%.

Predicted Amino Acid Sequence

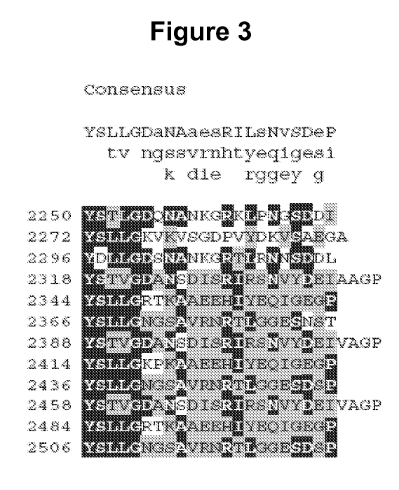

The ibpA ORF was predicted to encode 4,095 amino acid (aa) residues, with a calculated molecular weight of 450,052. The isoelectric point calculated from the predicted amino acid sequence was 5.37. Using the program SOSUI (23), the derived amino acid sequence of the ORF was predicted to be that of a soluble protein with an average hydrophobicity of −0.699 and with no long hydrophobic transmembrane domain. The predicted amino acid sequence contained nine cysteines, five of which were in the N-terminal putative signal peptide. Therefore, the flexibility of the polypeptide chain is not limited by disulfide bonds. The features found in the N-terminal sequence of ibpA include an unusually large putative signal peptide sequence at aa 1 to 97, similar to that found in Bordetella pertussis-filamentous hemagglutinin (FHA) precursor protein FhaB (24). This region also includes a 22-residue-long sequence highly preserved among secreted proteins of various other Gram-negative pathogens, a positively charged region (aa 41-55), and a hydrophobic segment (aa 56-91) as well as a putative cleavage site aa after residue 97 (S). The putative cleavage site fulfilled the (−6, −3, −1) rule pertaining to protein precursors processed by peptidase of the Lep type (25) Amino acid residue repeats of the same pattern with the nucleotide repeats were identified. In addition, approximately 22 residue repeats were revealed in the region of the first set of approximately 200-bp nucleotide repeats (FIG. 3). In FIG. 3, the amino acid sequence of the approximately 22-residue repeats from the approximately 200-bp repeats of the ibpA ORF and the consensus sequences. The major consensus sequences shown in upper case (5 or more of 12 residues) are shaded in black and the other consensus sequences shown in lower case are shaded in grey. These 22 residue repeats were of note because of their possible functional role as discussed below.

The predicted protein product of the ibpB ORF comprised 586 aa and had a calculated molecular weight of 66,383. Signal P (26) predicted that N-terminus of the ibpB had characteristics of prokaryotic signal sequence and the most likely cleavage site was between aa 30 (A) and 31 (Q). The presence of a C-terminal phenylalanine (F) is consistent with the prediction that the ibpB gene product is an integral outer membrane protein (27).

Similarity of ibpA and ibpB to other bacterial proteins

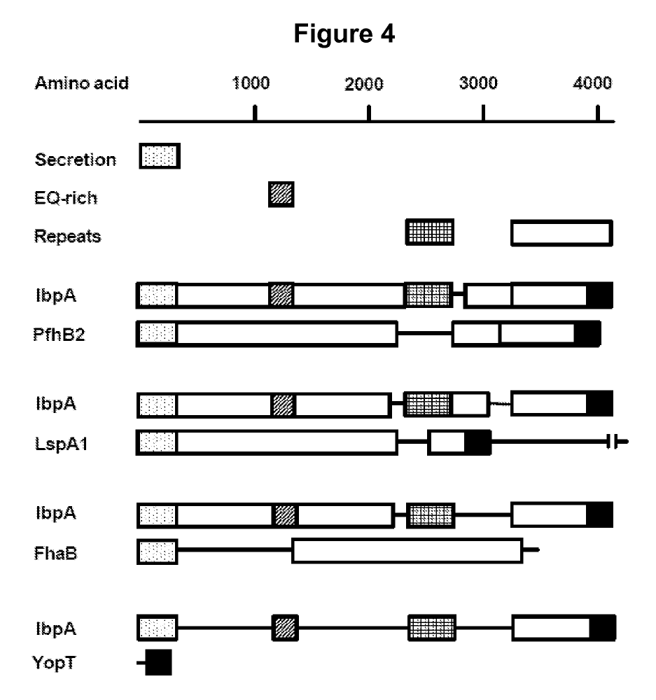

A BLAST search (28) of the combined nonredundant GenBank/EMBL/DDBJ protein databases revealed that the primary amino acid sequences of the predicted ibpA and ibpB proteins demonstrated homology to large exoproteins and their transporter proteins of Gram-negative bacteria that belong to the two-partner secretion pathway family (29). The large predicted or confirmed exoproteins related to ibpA include P. multocida PfhB1 (2,615 aa) and PfhB2 (3,919 aa) (22), Haemophilus ducreyi LspA1 (4,152 aa) and LspA2 (4,919 aa) (30), B. pertussis FhaB (3,591 aa) (31, 32) and others. Homologous regions among some of these proteins are summarized in FIG. 4.

In FIG. 4, boxes indicate homologous sequences between two predicted amino acid sequences demonstrated by a BLAST homology search. Shaded areas indicate putative functional regions or p76 gene region: dotted, secretion domain; slashes, EQ-rich domain; crosshatched, repeat regions corresponding to 210- and 285-bp repeating units; grey, p76 gene region; black, YopT family conserved region.

The greatest identity and similarity was observed with PfhB2 (53.6% and 61.4%, respectively). The similarity extended over the entire deduced amino acid sequences, however, a region with abundance of glutamic acid (E) and glutamine (Q) residues (EQ-rich domain, see below in this section) and the repeat regions corresponding to the 210- and 285-bp repeating units are unique in ibpA. The N-terminal secretion domain of approximately 400 aa was conserved among several large exoproteins (FIG. 4). Two secretion motifs, NPNL and NPNGI found in a family of secreted proteins (29) were conserved also (Table 1). The ibpA C-terminal region of approximately 200 aa residues is also homologous with YopT of Yersinia spp (33) and the sequence is conserved in the LspAl and LspA2 of H. ducreyi as well as the PfhB1 and PfhB2 of P. multocida but it is not present in the FhaB of B. pertussis (FIG. 4).

ibpA was demonstrated to have homologous regions to FhaB as shown in FIG. 4. Low level homology regions were detected for several known functional domains such as heparin binding domain (aa 525-975 of ibpA and aa 442-863 of FhaB) (34), and the carbohydrate recognition domain (aa 1307-1471 of ibpA and aa 1,141-1,279 of FhaB) (35) at 40.8% and 39.3% similarity respectively, as calculated based on sequence comparison data determined by the BESTFIT program (36). The FhaB hemagglutination domain consists of the C-terminal region of the heparin binding domain of FhaB and FhaB aa 1,655-2,111 (37). The N-terminal half of the whole ibpA sequence was overlapped in part with FhaB aa 1,655-2,111. Motifs demonstrated in ibpA are shown in Table 1. Some of these motifs are conserved among large exoproteins examined. The integrin binding motif, RGD, was unique in ibpA and FhaB but the TK-D sequence, with a possible role in integrin recognition (38), was found in all related large exoproteins listed in Table 1.

A BLAST homology search demonstrated a domain in the ibpA with homology to streptococcal M proteins at aa 1,112 to 1,255. The predicted 4,095 aa residues of ibpA were searched using the program COILS (39) because M proteins are known to have a coiled-coil α-helix structure (40). A highly predicted coiled-coil α-helix structure was demonstrated at aa 1,116-1,255 of ibpA. This region had an abundance of glutamic acid (E) and glutamine (Q) residues, so was designated as an EQ-rich domain. At the ibpA EQ-rich domain, homology was detected with Streptococcus pyogenes Enn18, Enn64/14 and Sir22 IgBPs. These three S. pyogenes IgBPs have been demonstrated to have a highly conserved 35 aa IgG3 binding sequence within an EQ-rich central conserved core region of each protein (41). Further comparison of surface IgBPs of S. pyogenes with the ibpA protein sequence demonstrated alignment of the IgG3 binding consensus sequence and ibpA at variable aa positions within aa 1,139-1221. Thus, the ibpA EQ-rich domain appears to include Ig-binding sequences like S. pyogenes IgBP EQ-rich domains. In addition to ibpA EQ-rich domain, a homologous sequence to one of the two binding regions of the IgA-binding β antigen of S. agalactiae (52) was found at aa 3,354-3,698 of ibpA.

Role of Putative Adhesin Motifs in Attachment of H. Somni to Bovine Endothelial Cells

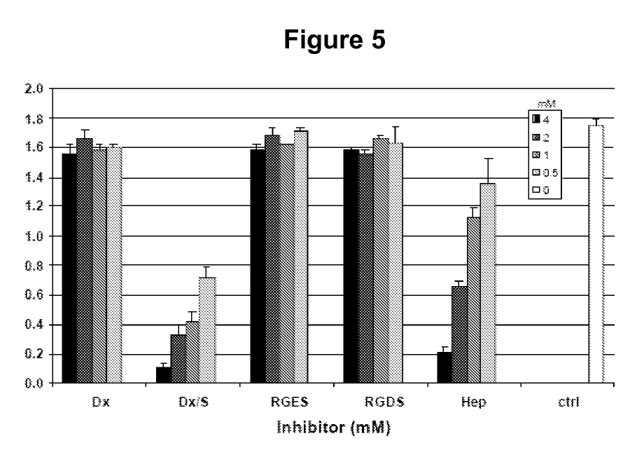

Competitive inhibition studies were done to test the role of adhesin motifs in binding of H. somni to bovine pulmonary artery endothelial cells because H. somni causes vasculitis of medium to large arteries in pneumonic lung. Adhesion to the target endothelial cells is likely to be the first step in pathogenesis. No inhibition of attachment was detected with the RGDS peptide in comparison with the negative control peptide, RGES (FIG. 5). However, when heparin or dextran sulfate (a comparable sulfated polysaccharide) was compared to the non-sulfated control, dextran, substantial inhibition was noted (FIG. 5). A dose response was detected, even down to 0.5 mM.

In FIG. 5, inhibitors include RGDS and its control peptide, RGES, as well as heparin (Hep) or the comparable sulfated polysaccharide, dextran sulfate (Dx/S) and its control, dextran (Dx). A control with no inhibitor (ctrl) is also included.

Demonstration of an Ig-Binding Region

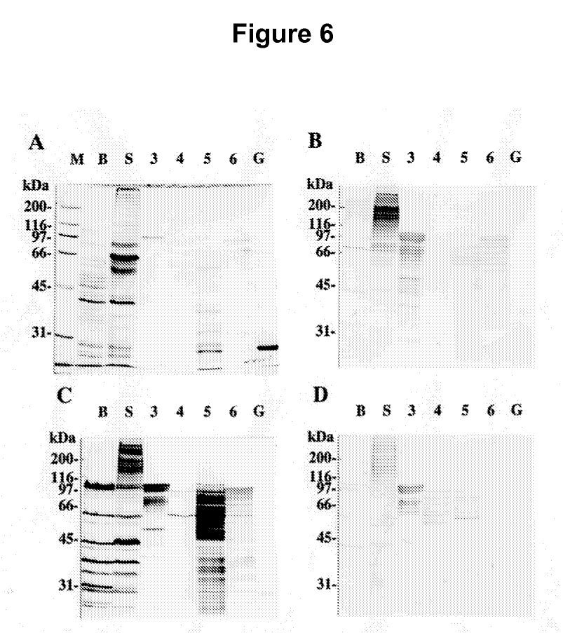

In order to localize the bovine IgG2 Fc-binding domain of ibpA, a series of expression plasmids for various GST-fused recombinant fragments was constructed but successful expression was observed only from pGST-ibpA3 (aa 972-1,515), pGST-ibpA4 (aa 1,516-2,070), pGST-ibpA5 (aa 2,070-2,730) and pGST-ibpA6 (aa 2,731-3,201). Western blot analysis of these purified GST-fused truncated fragments with bovine anti-DNP IgG2 revealed that GST-ibpA3 (aa 972-1,515), which includes the EQ-rich domain, reacted most strongly (of the fragments tested) with bovine IgG2 anti DNP (FIG. 6B).

In FIG. 6, antigens include H. somni 2336 whole bacteria (lane B), H. somni 2336 culture supernatant (lane S) and purified GST-ibpA fragments corresponding to amino acid sequence positions at 972-1,515, ibpA3, (lane 3); 1,516-2,070, ibpA4, (lane 4); 2,070-2,730, ibpA5, (lane 5) and 2731-3,201, ibpA6, (lane 6) as well as purified GST (lane G) as a control antigen. Antigens were electrophoresed on 12% SDS-PAGE low-bis gels and electrotransferred to nitrocellulose membranes. (A) Coomassie brilliant blue-stained gel. (B) Blot reacted with bovine anti-DNP IgG2 at 100 μg/ml to assess Fc binding. (C) Blot reacted with calf convalescent serum at 1:400 dilution, from calf #9001 with experimental H. somni pneumonia. (D) Blot reacted with calf preinfection serum 9001 at 1:400 dilution.

Taking the above results together, the EQ-rich domain appears to be a primary contributor to the Ig-binding activity of ibpA. Since there was no expressed GST-fused recombinant fragment of the 3,354-3,698 aa region homologous with the IgA binding protein of S. agalactiae, its role in Ig binding was not tested in the experiment reported above. However, this sequence was included in our previous study showing that the p76 peptide was an IgBP (18). Thus, putative Ig binding domains are found in both the N-terminal portion and the C-terminal portion of the ibpA molecule.

Immune Responses to the Truncated GST-ibpA Fragments

Western blots were done with pre- and post-infection sera from calves with H. somni induced pneumonia in a previous experiment (42). GST-ibpA3 and GST-ibpA5 reacted weakly with the control, pre-infection serum (perhaps due to IgG2 Fc binding) but strongly with the convalescent, post-infection serum (FIG. 6 C and D). This indicates that the calf did have a humoral immune response to ibpA3 and ibpA5. Convalescent phase serum also reacted with many other bands in H. somni whole cell pellets and in the culture supernatant fraction, as would be expected. In the latter, the high molecular weight (HMW) bands corresponding to HMW-IgBPs (17), reacted strongly. The peptides encoded by GST-ibpA3 and GST-ibpA5 were of lower MW than the HMW bands in the H. somni supernatant, but the truncated recombinant IgBPs were of the same multibanded character as the native IgBPs.

Preliminary Protection Experiment

In order to gain preliminary information on immune protection by ibpA3, a very small number of calves were immunized and challenged. Calves 9902 and 9906, vaccinated with the purified GST-ibpA3 fragment (designated “immunized calves”), survived the intrabronchial challenge with H. somni, showing only mild clinical signs of respiratory disease up to days 3-4 after challenge and then no clinical signs (Table 2). Of two control calves vaccinated with the purified recombinant GST protein alone, calf 9904 died on day 3 after challenge with marked severe respiratory disease. Control calf 9908 survived but showed severe to moderate clinical signs by day 2 after challenge. At necropsy, H. somni CFU numbers were more than ten times less in lung lesions of immunized calves than in those from control calves. Also, the lung lesion area was smaller in immunized calves than in control calves (mean % of pneumonic lung=7 vs. 34). An antibody response to H. somni HMW-IgBPs in concentrated culture supernatant was detected by Western blotting with serum from immunized calves (Table 2). Immunization of calves with GST protein elicited an antibody response to GST, which cross-reacted weakly with GST-ibpA3 but not with H. somni HMW-IgBPs. There was no detectable antibody to GST-ibpA3 or GST in sera of calves prior to immunization. From these results, it appears that the immune response to HMW-IgBPs detected in calves immunized with the GST-ibpA3 was protective against intrabronchial challenge with H. somni.

### Example 2

### Protection of Mice Against H. Somni Septicemia by Vaccination With Native IgBPs or Recombinant Subunits of Immunoglobulin Binding Proteins (IgBPs)

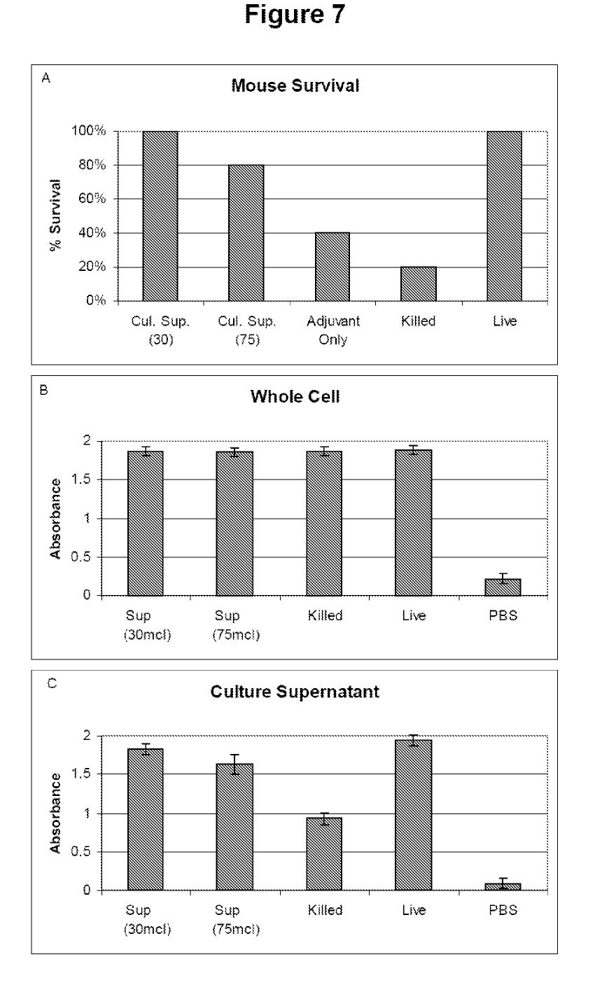

A mouse model of H. somni septicemia was used to test protective effects of IgBPs. Five to six week old NIH Swiss Webster female mice were immunized with modified H. somni culture supernatant (primarily IgBPs, a 60 K shed antigen and outer membrane blebs) or killed cells in Quil A adjuvant. Adjuvant alone was the negative control and live H. somni (convalescent mice) was the positive control. All were immunized 2× at a 3 week interval and were challenged 2 weeks after the second immunization. The modified culture supernatant at 30 or 75 μl per mouse protected as well as the live bacteria but the formalin killed H. somni protected no better than adjuvant alone (FIG. 7a).

In FIG. 7, mice (5/group) were vaccinated twice with culture supernatant at 30 μl or 75 μl, killed cells (2×108), live organisms (1×108), or Quil A adjuvant only. Two weeks after the second immunization, mice were challenged with 1×108 virulent H. somni organisms mixed with FCS. Percent survival is shown in A and the level of serum antibodies against the whole cell antigen (B) or culture supernatant antigen (B) by ELISA. Mouse serum was diluted 1:10,000.

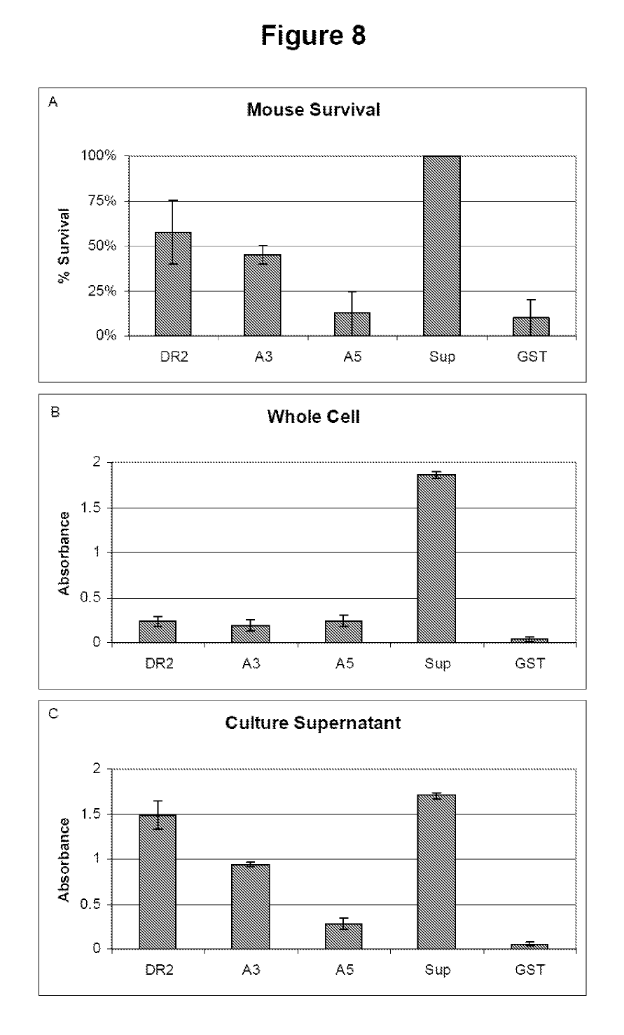

Antibody analysis by ELISA indicated that all vaccines induced high antibody responses against whole cell antigen but only live H. somni (convalescent mice) or the culture supernatant induced antibody levels above an absorbance of 1.0 (FIGS. 7b & c). Since the culture supernatant containing IgBPs was protective, we then tested the ability of recombinant IgBP subunits (approximately 40 μg of A3, A5 or DR2) to protect (see Tagawa et al. Microbial Pathogenesis 39:159-170, 2005). The experiment was done twice and results pooled. Again, the culture supernatant protected almost all mice and the negative control (GST and Quil A or Quil A alone) did not protect (FIG. 8a).

In FIG. 8, Mice (4-5/group; 2 separate trials) were vaccinated twice with recombinant IgBP subunits (approximately 40 μg of A3, A5 or DR2), culture supernatant at 30 μl, or Quil A adjuvant with GST. Two weeks after the second immunization, mice were challenged with 1×108 virulent H. somni organisms mixed with FCS. Percent survival is shown in A and the level of serum antibodies against the whole cell (B) or culture supernatant (C) by ELISA. Mouse serum was diluted 1:10,000.

The culture supernatant induced high levels of antibody against either whole cells or culture supernatant antigen but the recombinant peptides only induced antibody reactive with the culture supernatant antigen in ELISA (FIGS. 8b and c). The level of antibody response to each of the 3 recombinant peptides was directly related to the amount of protection. It may be that larger amounts of recombinant peptides would protect better or it may be that full length native IgBPs, the 60 K antigen and outer membrane blebs protects better due to the wider complement of protective epitopes. At any rate, it is clear that vaccination with culture supernatant (prepared under our specific protocol) is very effective at protecting against septicemia.

### Example 3

### Protection of Calves Against Histophilus Somni Induced Pneumonia By Vaccinating With Recombinant IbpA Subunits

Histophilus somni is one of the common causes of the Bovine Respiratory Disease (BRD) complex yet protection by vaccines is controversial. Although many whole cell vaccines protect against thrombotic meningoencephalitis, protection against pneumonia is less well documented. Some field studies show medium protection, others show no protection. Furthermore, adverse reactions to whole cell vaccines are a sometimes seen. To address these problems, we developed a bovine vaccine composed of recombinant subunits of the virulence factor (and antigen) IbpA or Immunoglobulin Binding Protein (IgBP) found on the surface of virulent H. somni.

Methods:

Calves: Healthy 5-6 week old calves were received from a nearby dairy, stabilized for a few days and vaccinated with recombinant subunits as GST fusion proteins (200 micrograms per dose) or with culture supernatant (positive control at 150 microliters of a 10× concentrated dialysed supernantant ) or GST alone (negative control at 67 micrograms which equals the amount of GST in 200 micrograms of fusion protein). Each of the five treatment groups consisted of 6 calves. Each dose in 1 ml was emulsified with 1 ml of Complete Freund's Adjuvant and given subcutaneously. Three weeks later calves were immunized a second time with the same dose but in Incomplete Freund's Adjuvant. Two weeks after the last vaccination, calves were challenged intrabronchially with 5×109 Colony Forming Units (CFU) of H. somni strain 2336. Clinical signs were monitored before and throughout the immunization and twice daily after challenge. Calves were necropsied at day 4 after challenge by a veterinary pathologist (Lisa LaFranco, DVM, PhD. ACVP Diplomate), assisted by the rest of the research team. Gross lung lesion volumes were estimated by a standardized protocol and samples were taken for bacterial culture and histopathology. Samples for viral serology, H. somnus antibody assays were taken throughout the experiment.

Recombinant IbpA Subunit Fusion Protein Production:

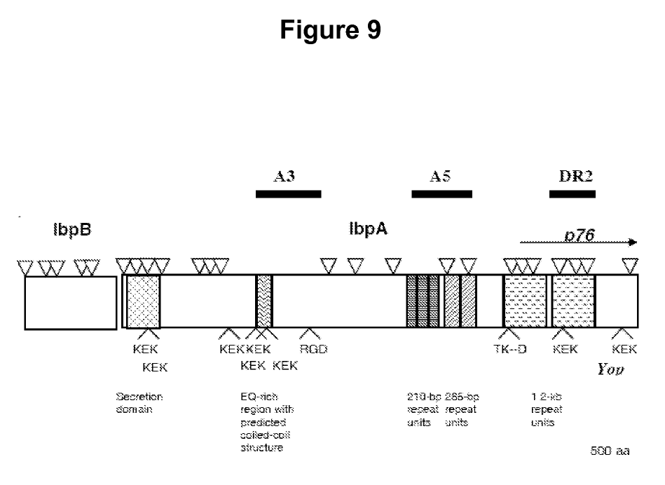

The approximate location of each of the IbpA subunits is shown in FIG. 9.

In FIG. 9, multiple ATG translational start sites are shown by small triangles, the p76 gene with a horizontal arrow (labeled p76) and RGD, TKXXD and KEK motifs by open arrows. Sets of repeats are shaded similarly. The 1.2 kb repeat units (DR1 & DR2) are upstream from the cysteine proteinase catalytic domain near the C terminus. Apporximate locations of IbpA3, IbpA5 and IbpA DR2 are indicated.

Previously described plasmids pHS 140 and pHS 134 were used for cloning the A3 and A5 coding regions of the IbpA gene of Histophilus somni. pHS 139 was used as a template for the DR2 sequence.

The forward and reverse primers for A3:

(SEQ ID NO: 18)

AGCTGGATCCAGTGAACGAATCACTGTAGG,

(SEQ ID NO: 19)

AGCTGAATTCGAAGCTTTCACAAAACCTGTTTTA;
A5:

(SEQ ID NO: 20)

AGCTGGATCCGATTTGGATCTTGTAGCGG,

(SEQ ID NO: 21)

AGCTGAATTCGAGGCATAAATATGATCTGCCG;
for DR2:

(SEQ ID NO: 22)

AGCTGGATCCATCGAAAAGTTAAATCATGGATTA,

(SEQ ID NO: 23)

AGCTGAATTCAGATTATTTTTTTTGTAGTTGACCAC.

The PCR-amplified A3 and A5 fragments were ligated into pET-GSTx and the amplified DR2 fragment was ligated into pET41a (Novagen, Darmstadt, Germany) using the Rapid DNA Ligation Kit (Roche, Nutley, N.J.). Both vectors encoded a glutathione-S-transferase (GST) tag at the N-terminus of the inserted DNA sequence, and a His tag at the C-terminus. Escherichia coli BL21 DE3 codon plus cells transformed with each of the plasmids was used to innoculate 1 liter of LB media containing ampicillian (GSTx) or kanamyacin (pET41a) and chloramphenicol and were grown at 37° C. to an OD600 of 0.6, at which time protein expression was induced by addition of 0.4 mM isopropyl-D-thiogalactopyranoside (IPTG). After induction the culture was left at 25° C. ON and harvested by centrifugation. The cells were resuspended in 50 ml of PBS containing 1% triton and 5 mM DTT (lysis buffer) and were lysed using a French Press. The lysate was cleared by centrifugation at 15,000×g for 30 min at 4° C. and the GST tagged proteins were attached to the glutathione agarose (Sigma, San Louis, Miss.) by rotation for one hour at 4° C. The protein-loaded beads were washed by rotation at 4° C. with lysis buffer (4×10 ml) and wash buffer, 50 mM Tris pH 8.0, 100 mM NaCl, 5 mM DTT, (3×10 ml) for 10 minutes each wash. The protein was eluted twice with 20 mM glutathione in wash buffer for 30 min at 40 C. Protein concentrations were determined with the BioRad Protein Assay (BioRad Laboratory Inc., Hercules, Calif.). Typically, 5-10 μg of protein was added to SDS-PAGE loading buffer followed by denaturation for 5 min at 100° C. and separation using SDS-PAGE electrophoresis. Proteins were visualized using Coomassie staining.

Antibody Assays:

Serum was collected before immunization and throughout the experiment. Bronchio-alveolar secretions were flushed at necropsy. Isotypic antibody levels were quantitated by ELISA against whole cells, culture supernatant, and recombinant IbpA fusion proteins (A3, A5 and DR2) as well as the negative control antigen, GST.

Results:

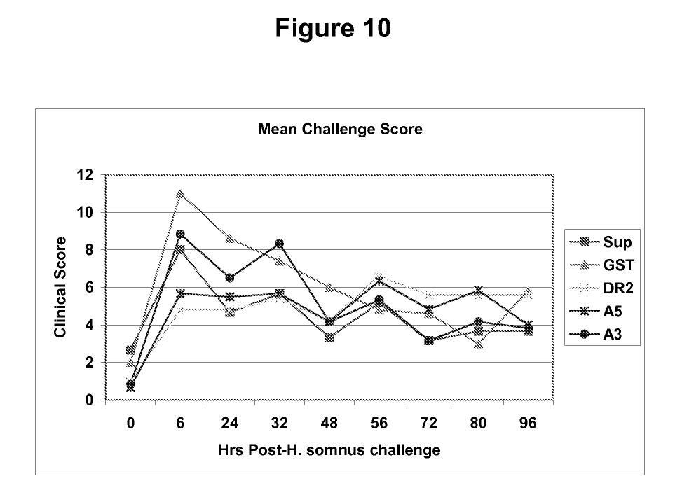

Clinical scores were recorded twice a day after challenge. Peak scores were usually obtained within 6 hours but the calves returned to approximately a score of 6 or less by the next day except for the GST immunized negative control group and perhaps the IbpA3 immunized group (FIG. 10). In FIG. 10, a score of 6 or less was considered to be mild and 12 was very severe.

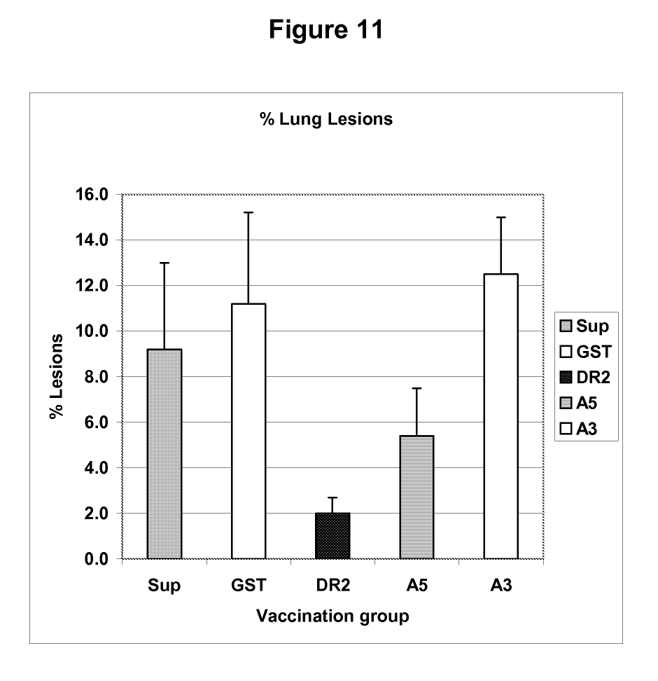

The DR2 immunized group had the lowest clinical scores at 6 hours after challenge and generally stayed below a clinical score of 6 (the cutoff). The volume of lesions (expressed as percentage of the lung at necropsy or “percent lesions”) was a more revealing measure of protection (FIG. 11). The IbpA subunit DR2 vaccinated animals had a significantly lower amount of pneumonic lung than the GST control immunized animals (p<0.05) even though the variability was quite large. The positive control group (culture supernatant immunized) was not significantly different from the negative control group (GST immunized). This positive control supernatant antigen protected mice very well, so it is not clear why the positive control calves were not well protected. In any case, The DR2 group was protected in that their lung lesions were significantly lower than the negative control vaccine group and protected better than the positive control antigen.

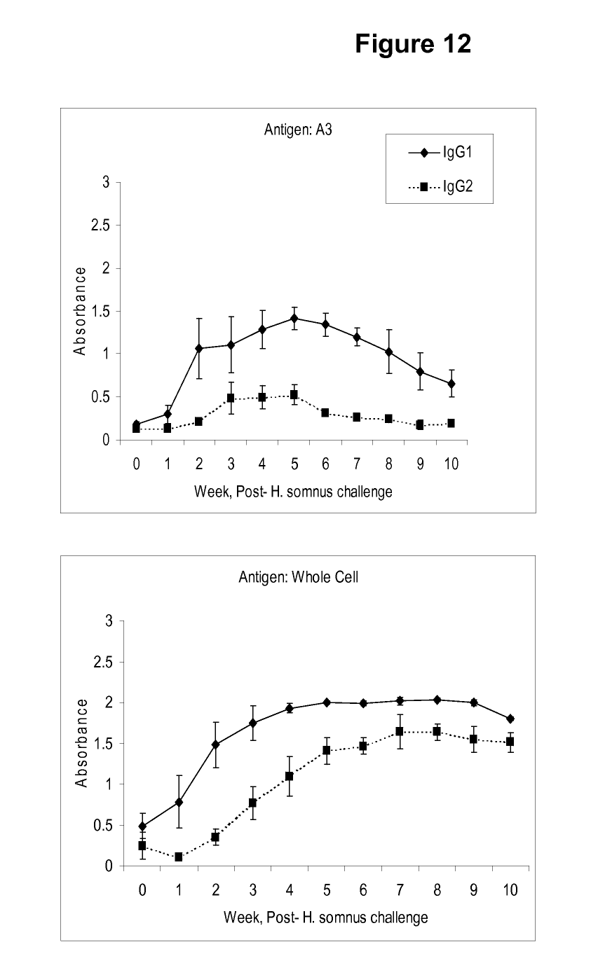

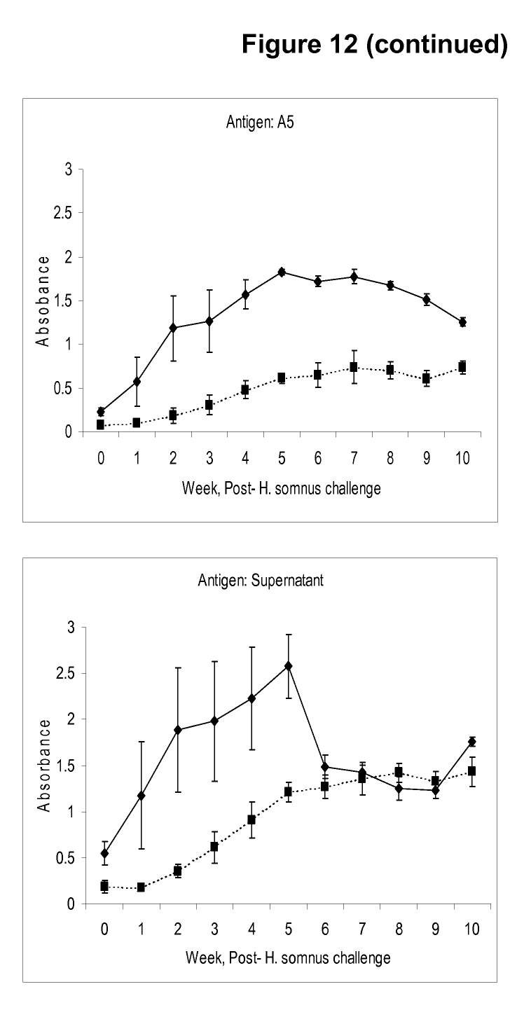

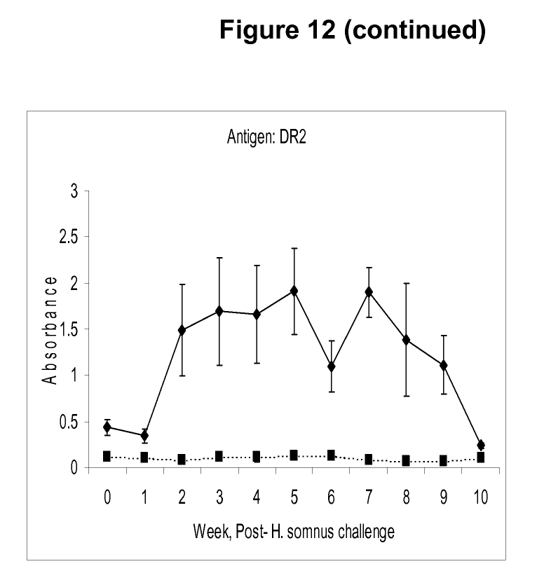

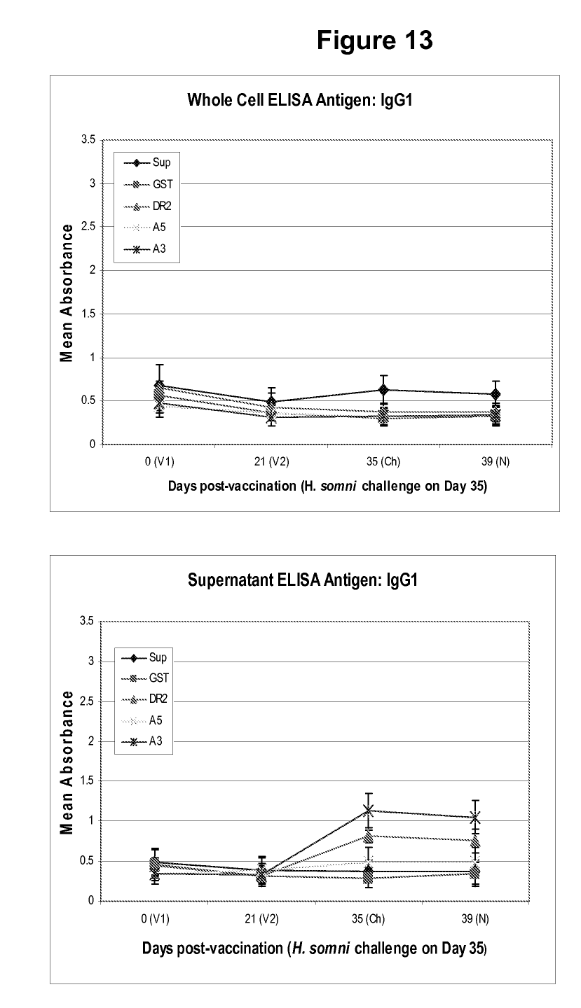

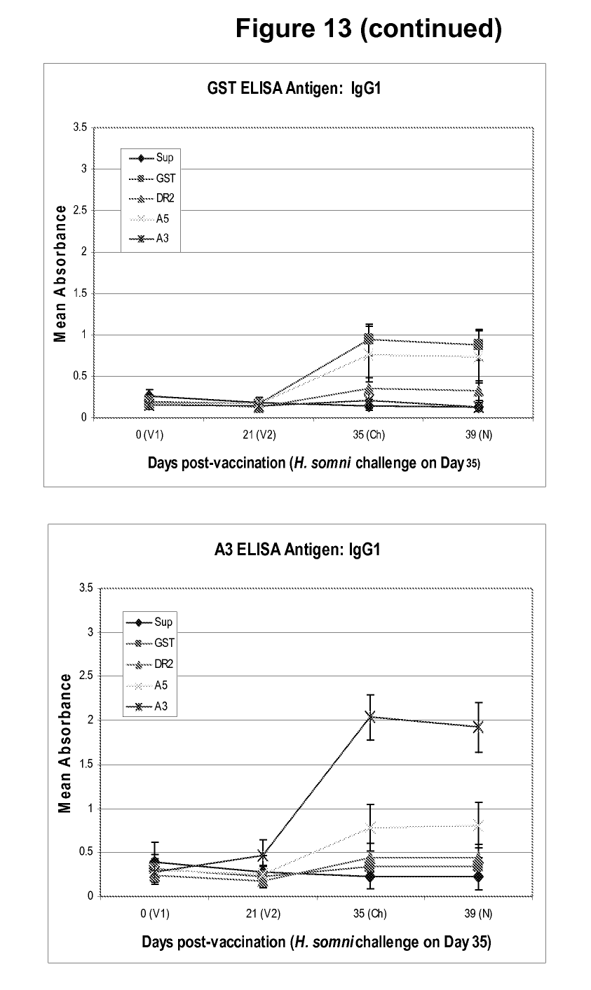

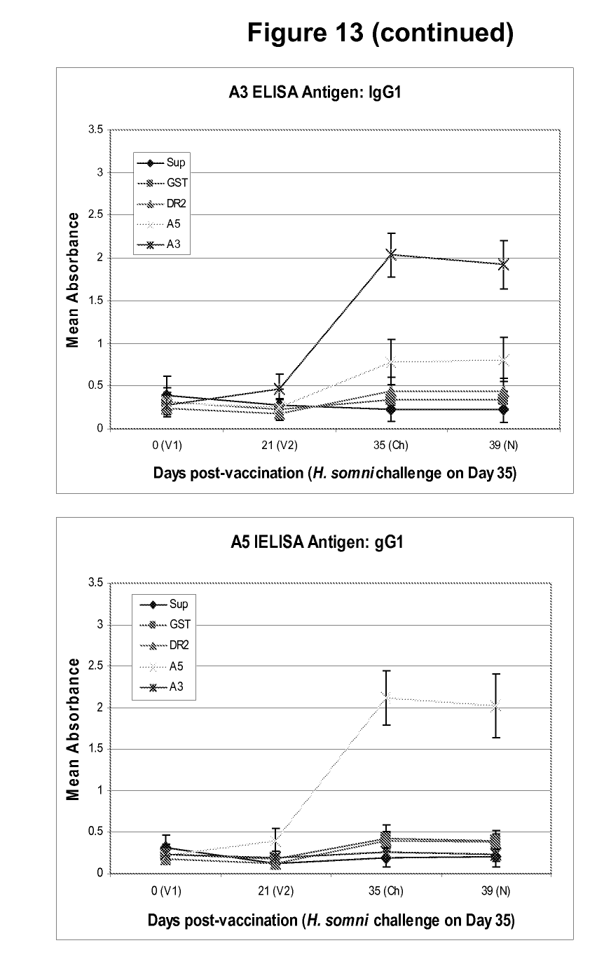

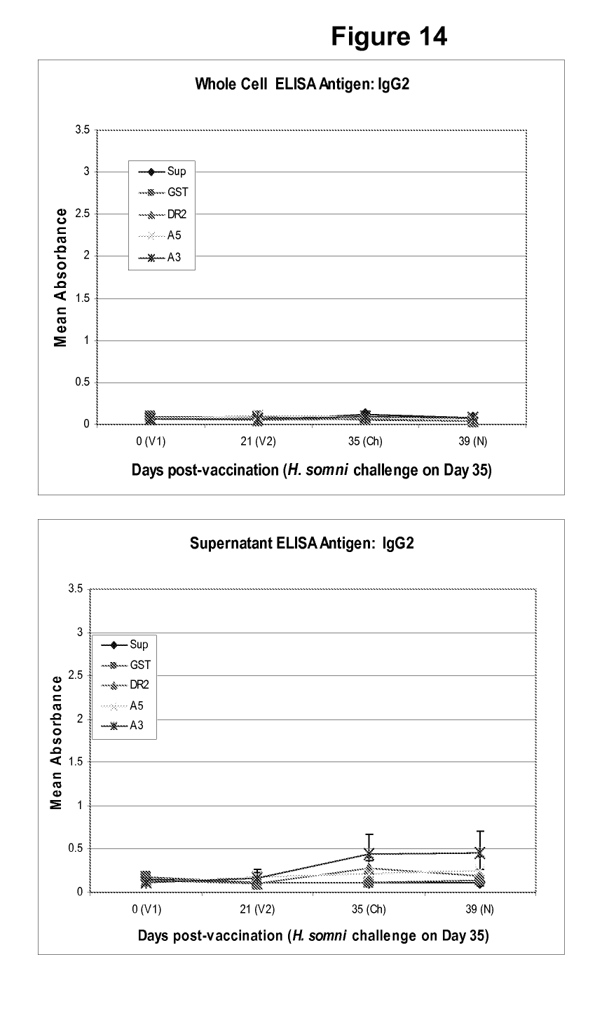

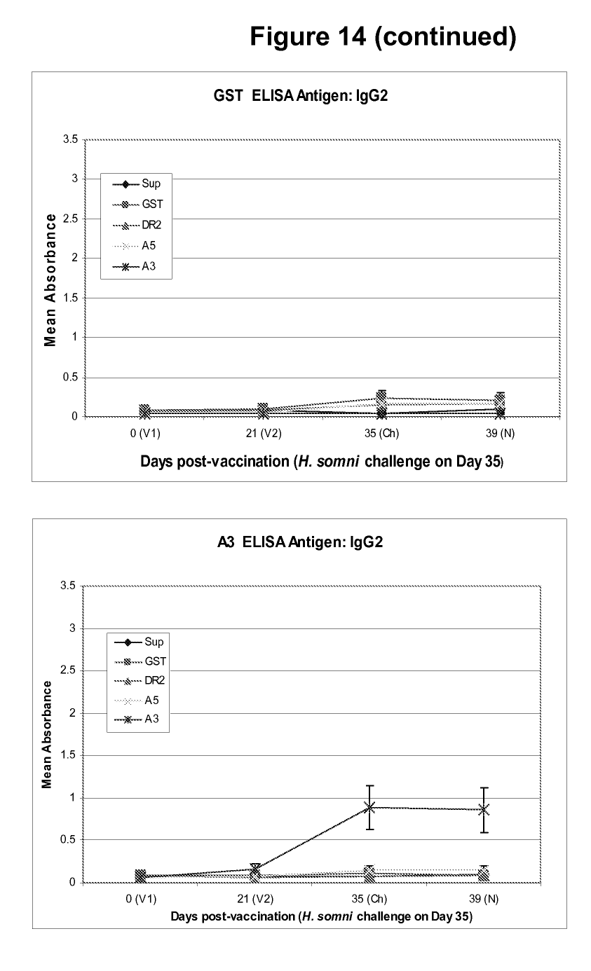

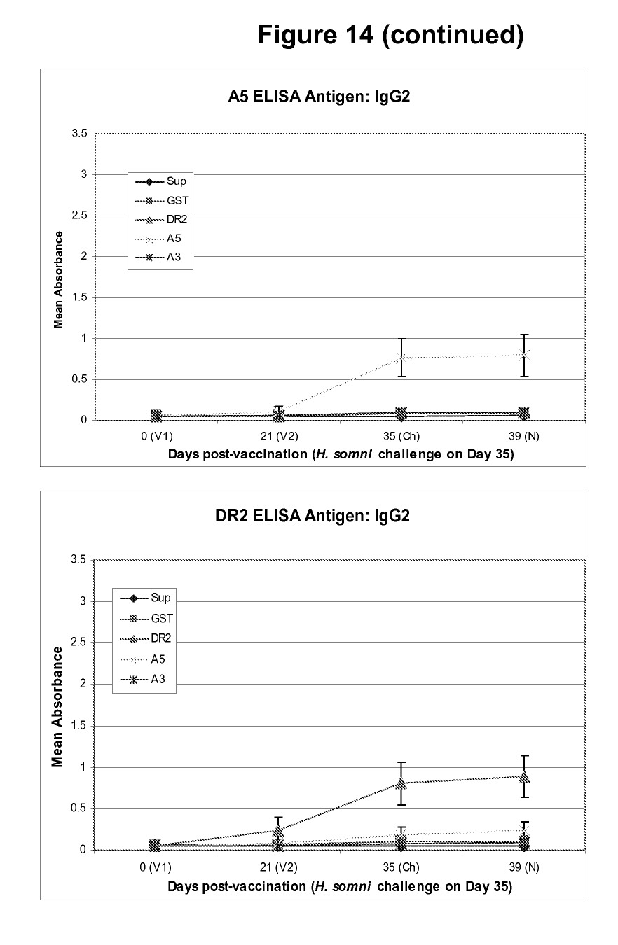

Antibody studies confirmed that the protection was related to specific antibodies. Both IgG1 and IgG2 antibodies were specific for the immunizing antigen in each group. (FIGS. 12, 13 and 14). The IgG1 antibody level was highest for the DR2 immunized group. IgG2 levels were similar in each group for the immunization antigen in each case. Interestingly, the group immunized with supernatant did not respond well to the supernatant antigen on the ELISA plate. In case this was due to use of too low an antigen dose, we immunized two additional calves with 10 times more supernatant antigen than the amount used in the rest of the trail. The antibody levels to supernantant antigen in those two calves were also low. The lack of a good antibody response in these positive control supernatant immunized calves is consistent with the poor protection of the same calves.

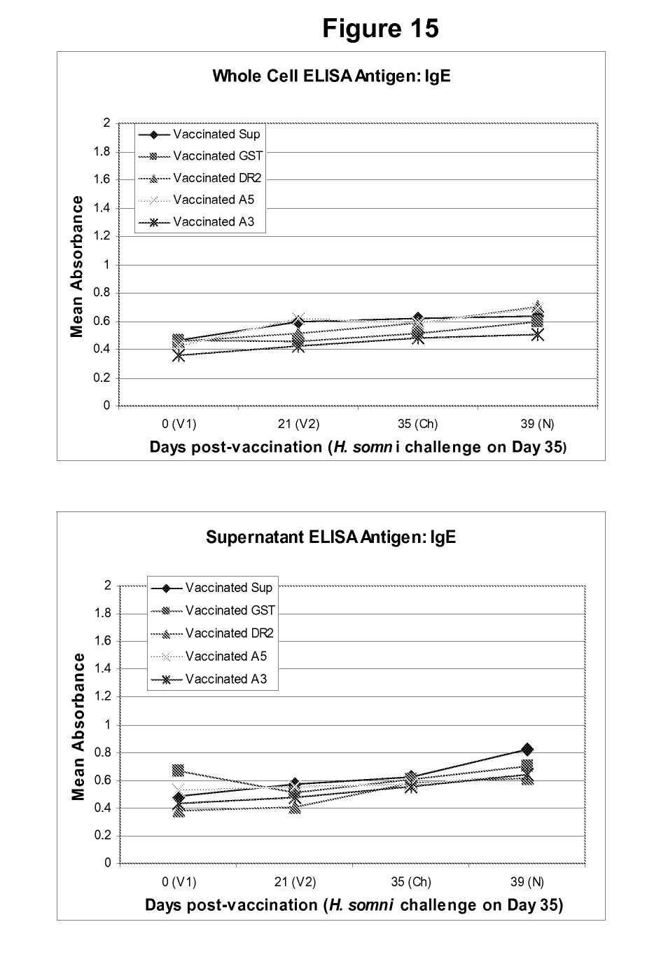

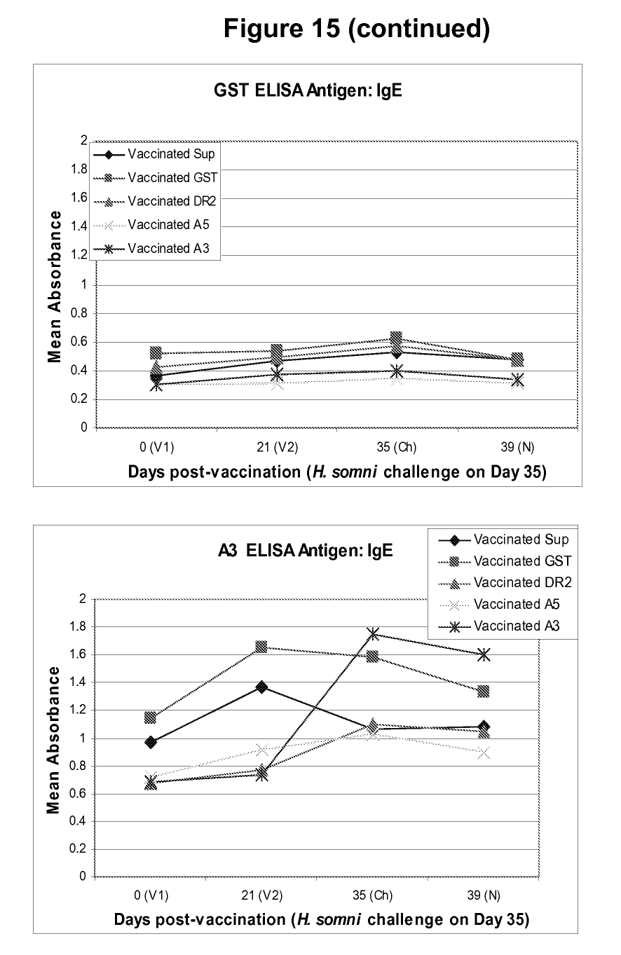

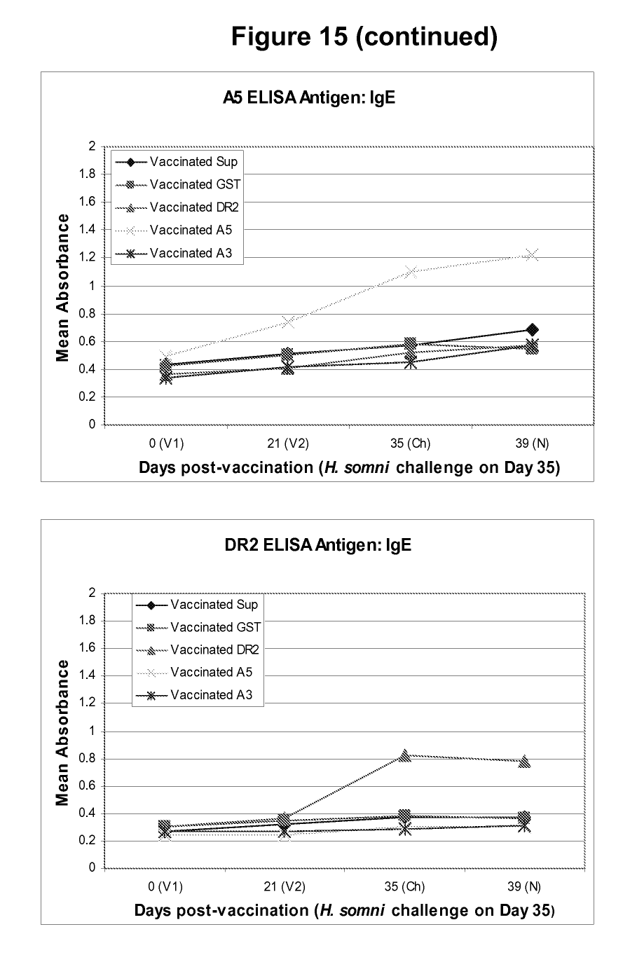

IgE antibody levels have been associated with adverse reactions and with worse clinical signs of longer duration in calves with H. somni pneumonia. Therefore, we measured IgE antibody levels in serum as well as the IgG responses. Although all groups had IgE responses to H. somni, the DR2 antigen elicited the lowest responses (FIG. 15).

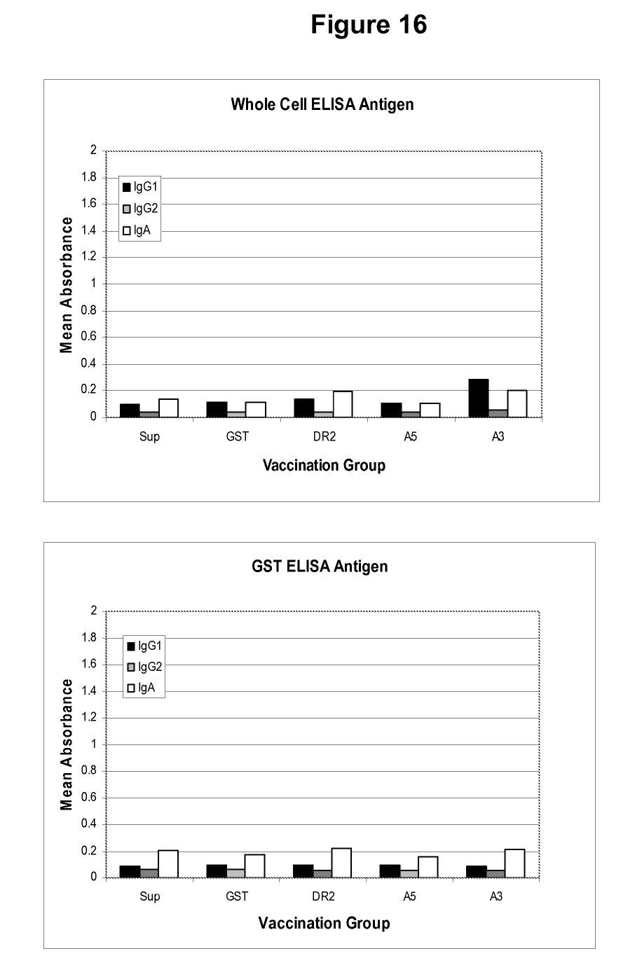

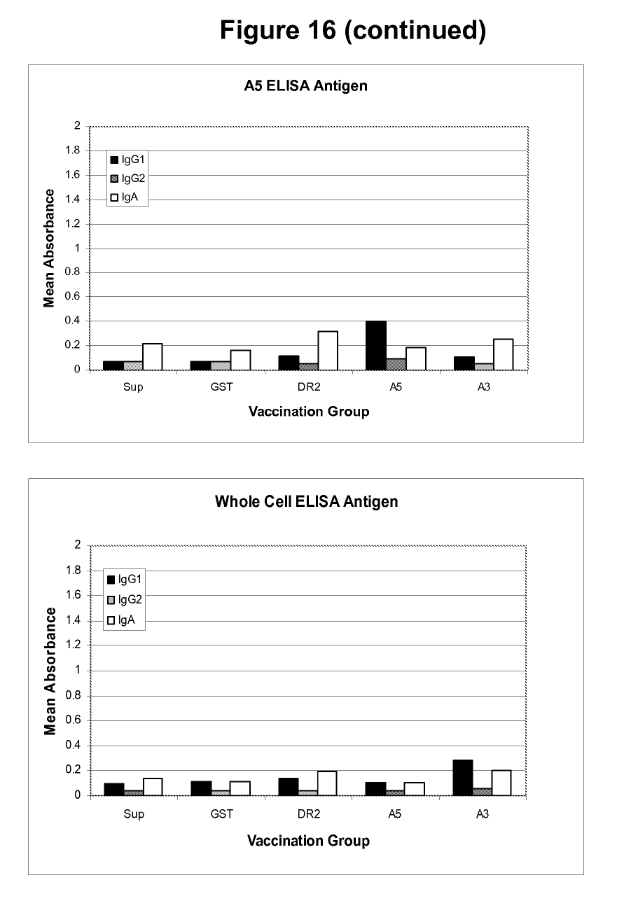

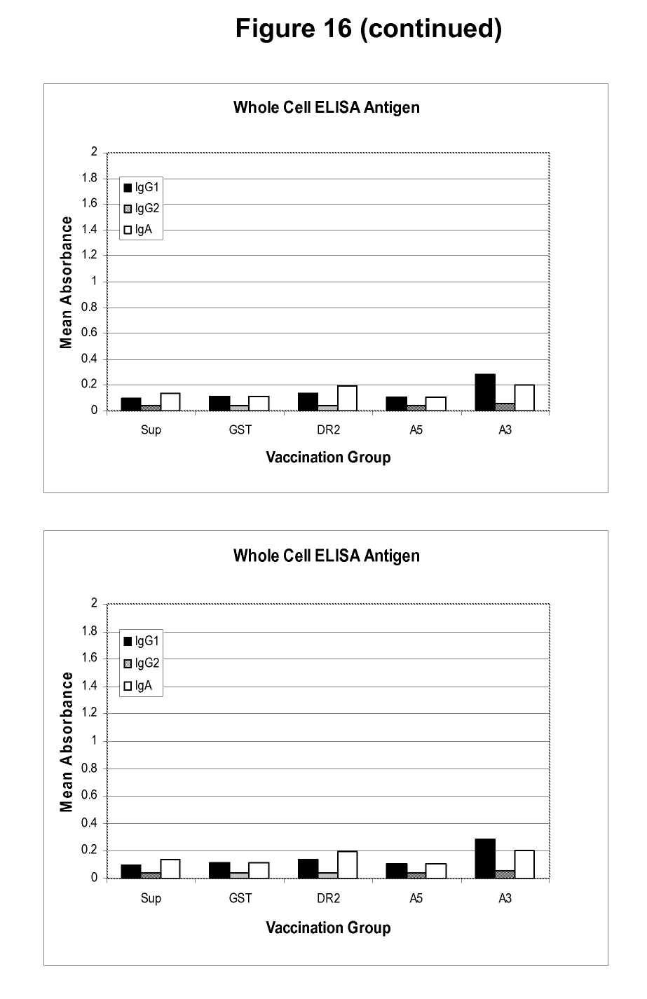

Antibodies (IgG1, IgG2 and IgA) in lung lavage fluid collected at necropsy was also assayed by ELISA against each of the standard antigens. Again, each immunization group had the highest ELISA readings when tested against the antigen used for immunization (FIG. 16).

Conclusions:

Immunization of calves with IbpA DR2 protects against H. somni induced bovine pneumonia. This protection is associated with specific serum IgG1 and IgG2 antibody responses and IgG1, IgG2 and IgA responses in bronchial secretions.

### Example 4

### Immune Responses of Infected Calves to H. Somni IbpA Subunits and Control Antigens

Isotypic antibody responses of calves with H. somni pneumonia were studied throughout the infection period to determine the kinetics of the IgG1 and IgG2 responses to IbpA subunits A3, A5, and DR2 as well as control whole cell and supernatant antigens. This establishes the relative responses of each isotype to each antigen. The results will contribute to the understanding of protective immunity, since 3 of the 5 calves were challenged at 10 weeks post infection with 10× the original dose and found to be resistant to pneumonia. The study should also provide insight into which antigens are most useful for immunodiagnosis.

Methods:

Calves: Healthy 8 -12 week old calves were obtained from nearby farms. After stabilization, each calf was infected intrabronchially with H. somni strain 2336. Serum was collected weekly for antibody analysis and bronchio-alveolar lavage fluid was cultured for H. somni. Clinical signs were monitored.

Antibody analysis: Serum antibody was quantitated by ELISA with IbpA subunits or control antigens on the solid phase and developed with anti-bovine IgG1 or IgG2 conjugates.

Results:

Calves responded to all antigens but backgrounds and kinetics differed (FIG. 9). Backgrounds were highest with the control whole cell and culture supernatant antigens for both IgG1 and IgG2 responses. Since these calves were between 8 and 12 weeks old, maternal immunity would had waned and active immunity was low. Therefore, the background antibody levels to whole cell and supernatant in serum of these calves should be lower than that of older calves, suggesting that the background in serum from older calves would be even higher. IgG2 antibody levels began to increase by week 2 but were never as high as IgG1 responses. Interestingly, there was no detectable IgG2 response to the IbpADR2 antigen. IgG1 levels increased by week 1 in all cases, except for the DR2 antigen. The A5 antigen had low backgrounds for both IgG1 and IgG2. It also detected a substantial increase in IgG1 antibody levels by week one. The IgG1 antibody level to the A5 antigen was almost as high the response to whole cells at week 2 and beyond but the background antibody level to A5 was much lower than with whole cell or supernatant antigens. Similar results were obtained with 24 additional calves in that IgG1 responses to the A5 antigen were as increased by week 2 as did the response to whole cells and supernatant antigens but without the high backgrounds with the latter two antigens.

Conclusions:

IbpA5 may be a useful diagnostic antigen when anti-bovine IgG1 conjugates are used to develop the assay.

TABLE 1
Motifs found among large exoproteins; ibpA of H. somni,
FHA of Bordetella pertussis, LspA1/LspA2 of Haemophilus ducreyi
and PfhB1/PfhB2 of Pasteurella multocida

| Motif | ibpA^{a} | FHA | LspA1 | LspA2 | PfhB1 | PfhB2 |
|---|---|---|---|---|---|---|
| NPNL | 198-201 | + | + | + | + | + |
| NPNGI | 238-242 | + | + | + | + | + |
| RGD | 1445-1447 | + | − | − | − | − |
| TK--D | 3029-3033 | + | + | + | + | + |
| | and | | | | | |
| | 3744-3748 | | | | | |

^{a}Residue numbers of predicted amino acid sequence of the ibpA ORF.

TABLE 2
Protection of calves immunized with the GST-ibpA3 against intrabronchial challenge of H. somni 2336

| | | | Intensity of antigen bands detected | | | | Result of H. somni challenge | | |
|---|---|---|---|---|---|---|---|---|---|
| | | | in Western blot^{a} | | | | | Isolation of | |
| | | | | | HMW-IgBPs | | Mean | H. somni from | Lung |
| Exp. | Calf | | GST- | | from | Challenge | clinical | lung lesion | lesion |
| No. | No. | Immunization | ibpA3 | GST | H. somni | dose | score | (CFU/g) | (%) |
| 1 | 9902 | GST-ibpA3 | +++ | + | +++ | 1.6 × 10¹⁰ | 3.9 | 5.2 × 10⁷ | 11 |
| | 9904 | GST | + | +++ | − | 1.6 × 10¹⁰ | 16.0 | 8.4 × 10⁸ | 54 |
| 2 | 9906 | GST-ibpA3 | +++ | + | +++ | 2.0 × 10¹⁰ | 2.1 | 1.3 × 10⁷ | 3 |
| | 9908 | GST | + | +++ | − | 2.0 × 10¹⁰ | 6.0 | 3.3 × 10⁸ | 14 |

^{a}Western blot was performed for sera collected on 0, 3, 6 and 8 weeks after first immunization. The antibody reactions to immunization antigens were not detected with sera on week 0. Immune responses to immunization antigens were detected and the data shown were for sera collected on week 8. Band intensity is as follows; +++: strong, ++: moderate, +: weak, and −: no reaction.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### Other References Include

Citation of a reference herein shall not be construed as an admission that such is prior art to the present invention. Specifically intended to be within the scope of the present invention, and incorporated herein by reference in its entirety, is the following publications: Tagawa et al. Genetic and functional analysis of Haemophilus somnus high molecular weight-immunoglobulin binding proteins. Microb. Pathog. 2005 November-December;39(5-6):159-70; and Geertsema et al. Bovine plasma proteins increase virulence of Haemophilus somnus in mice. Microb. Pathog. 2007 January;42(1):22-8. Other publications incorporated herein by reference in their entirety include:

1. Harris F W, Janzen E D. The Haemophilus somnus disease complex (hemophilosis): a review. Can. Vet. J. 1989; 30:816-822.

2. Humphrey J, Stephens L. Haemophilus somnus: a review. Vet. Bull. 1983; 53:987-1004.

3. Kwiecien J M, Little P B. Haemophilus somnus and reproductive disease in the cow. Can. Vet. J. 1991; 32:595-601.

4. Miller R B, Lein D H, McEntee K E, Hall C E, Shaw S. Haemophilus somnus infection of the reproductive tract of cattle: a review. J. Am. Vet. Med. Assoc. 1983; 187:1390-1392.

5. Stephens L R, Little P B, Wilkie B N, Barnum D A. Infectious thromboembolic meningoencephalism in cattle: a review. J. Am. Vet. Med. Assoc. 1981; 178:378-384.

6. Ward A C S, Corbeil L B, Mickelsen W D, Sweet V F. A selective medium for gram-negative pathogens from bovine respiratory and reproductive tracts. Am. Assoc. Vet. Lab. Diag. 1983; 26:103-112.

7. Ward G E, Nivard J R, Maheswaran S K. Morphologic features, structure, and adherence to bovine turbinate cells of three Haemophilus somnus variants. Am. J. Vet. Res. 1984; 45:336-338.

8. Thompson K G, Little P B. Effect of Haemophilus somnus on bovine endothelial cells in organ culture. Am. J. Vet. Res. 1981; 42:748-754.

9. Corbeil L B, Gogolewski R P, Stephens L R, Inzana T J. Haemophilus somnus: antigen analysis and immune responses, In Donachie W. et al. Eds. Haemophilus, Actinobacillus, and Pasteurella. New York: Plenum Press, 1995: 63-73.

10. Czuprynski C J, Hamilton H L. Bovine neutrophils ingest but do not kill Haemophilus somnus in vitro. Infect. Immun. 1985; 50:431-436.

11. Gogolewski R P, Kania S A, Inzana T J, Widders P R, Liggitt H D, Corbeil L B. Protective ability and specificity of convalescent serum from Haemophilus somnus pneumonia. Infect. Immun. 1987; 55:1403-1411.

12. Lederer J., Brown J F, Czuprynski C J. Haemophilus somnus, a facultative intracellular pathogen of bovine mononuclear phagocytes. Infect. Immun. 1987; 55:381-387.

13. Corbeil L B, Blau K, Prieur D J, Ward A C S. Serum susceptibility of Haemophilus somnus from bovine clinical cases and carriers. J. Clin. Microbiol. 1985; 22:192-198.

14. Inzana T J, Iritani B, Gogolewski R P, Kania S A, Corbeil L B. Purification and characterization of lipooligosaccharides from four strains of Haemophilus somnus. Infect. Immun. 1988; 56:2830-2837.

15. Widders P R, Dorrance L A, Yarnall M, Corbeil L B. Immunoglobulin-binding activity among pathogenic and carrier isolates of Haemophilus somnus. Infect. Immun. 1987; 57:639-642.

16. Yarnall M, Widders P R, Corbeil L B. Isolation and characterization of Fc receptors from Haemophilus somnus. Scand. J. Immunol. 1988; 28:129-137.

17. Yarnall M, Gogolewski R P, Corbeil L B. Characterization of two Haemophilus somnus Fc receptors. J. Gen. Microbiol. 1988; 134:1993-1999.

18. Corbeil L B, Bastida-Corcuera F D, Beveridge T J. Haemophilus somnus immunoglobulin binding proteins and surface fibrils. Infect. Immun. 1997; 65:4250-4257.

19. Cole S P, Guiney D G, Corbeil L B. Two linked genes for outer membrane proteins absent in four non-disease strains of Haemophilus somnus. Mol. Microbiol. 1992; 6:1895-1902.

20. Corbeil L B, Chikami G, Yarnall M, Smith J, Guiney D G. Cloning and expression of genes encoding for Haemophilus somnus antigens. Infect. Immun. 1988; 56:2736-2742.

21. Cole S P, Guiney D G, Corbeil. L B. Molecular analysis of a gene encoding a serum-resistance-associated 76 kDa surface antigen of Haemophilus somnus. J. Gen. Microbiol. 1993; 139:2135-2143.

22. May BJ, Zhang Q, Li L K, Paustian M, Whittam T S, Kapur V. Complete genomic sequence of Pasteurella multocida, Pm70. Proc. Natl. Acad. Sci. USA. 2001; 98:3460-3465

23. Hirokawa T, Boon-Chieng S, Mitaku S. SOSUI: classification and secondary structure prediction system for membrane proteins. Bioinform. 1998; 14:378-379.

24. Lambert-Buisine C, Willery E, Locht C, Jacob-Dubuisson F. N-terminal characterization of the Bordetella pertussis filamentous haemagglutinin. Mol. Microbiol. 1998; 28:1283-1293.

25. Pugsley A P. The complete general secretory pathway in gram-negative bacteria. Microbiol. Rev. 1993; 57:50-108.

26. Nielsen H, Engelbrecht J, Brunal S, vonHeijne G. Identification of prokaryotic and eukaryotic signal peptides and prediction of their cleavage sites. Protein Engin. 1997; 10:1-6.

27. Struyve M, Moons M, Tommassen J. Carboxy-terminal phenylalanine is essential for the correct assembly of a bacterial outer membrane protein. J. Mol. Biol. 1991; 218:141-148. 1991

28. Altschul S F, Madden T L, Schaffer A A, Zhang J, Zhang Z, Miller W, Lipman D J. Gapped BLAST and PSI-BLAST: a new generation of protein database search programs. Nucleic Acid Res. 1997; 25:3389-3402.

29. Jacob-Dubuisson F, Locht C, Antoine R. Two-partner secretion in Gram-negative bacteria: a thrifty, specific pathway for large virulence proteins. Mol. Microbiol. 2001; 40:306-313.

30. Ward C K, Lumbley S R, Latimer J L, Cope L D, Hansen E J. Haemophilus ducreyi secretes a filamentous hemagglutinin-like protein. J. Bacteriol. 1998; 180:6013-6022.

31. Domenighini M., Relman D, Capiau C et al. Genetic characterization of Bordetella pertussis filamentous haemagglutinin: a protein processed from an unusually large precursor. Mol. Microbiol. 1990; 4:787-800.

32. Relman D A, Domenighini M, Tuomanen E, Rappuoli R, Falkow S. Filamentous hemagglutinin of Bordetella pertussis: nucleotide sequence and crucial role in adherence. Proc. Natl. Acad. Sci. USA 1989; 86:2637-2641.

33. Shao F, Merritt P M, Bao Z, Innes R W, Dixon J E. A Yersinia effector and a Pseudomonas avirulence protein define a family of cysteine proteases functioning in bacterial pathogenesis. Cell 2002; 109:575-588.

34. Hannah J H, Menozzi F D, Renauld G, Locht C, Brennan M J. Sulfated glycoconjugate receptors for the Bordetella pertussis adhesin filamentous hemagglutinin (FHA) and mapping of the heparin-binding domain on FHA. Infect. Immun. 1994; 62:5010-5019.

35. Prasad S M, Yin Y, Rodzinski E, Tuomanen E I, Masure H R. Identification of a carbohydrate recognition domain in filamentous hemagglutinin from Bordetella pertussis. Infect. Immun. 1993; 61:2780-2785.

36. Devereux J, Haeberli, P, Smithies O. A comprehensive set of sequence analysis programs for the VAX, Nucleic Acids Res. 1984; 12:387-395.

37. Leininger E, Bowen S, Renauld-Mongenie G et al. Immunodominant domains present on the Bordetella pertussis vaccine component filamentous hemagglutinin. J. Infect. Dis. 1997; 175:1423-1431.

38. Rozdzinski E, Sandros J, van der Flier M et al. Inhibition of leucocyte-endothelial cell interactions and inflammation by peptides from a bacterial adhesin which mimic coagulation factor X. J. Clin. Invest. 1995; 95:1078-1085.

39. Lupas A, Van Dyke M, Stock J. Predicting coiled coils from protein sequences. Science 1991; 252:1162-1164.

40. Manjula B N, Trus B L, Fischetti V A. Presence of two distinct regions in the coiled-coil structure of the streptococcal Pep M5 protein: relationship to mammalian coiled-coil proteins and implications to its biological properties. Proc. Natl. Acad. Sci. USA 1985; 82:1064-1068.

41. Pack T D, Podbieski A, Boyle M D P. Identification of an amino acid signature sequence predictive of protein G-inhibitable IgG3-binding activity in group-A streptococcal IgG-binding proteins. Gene 1996; 171:65-70.

42. Tagawa Y, Haritani M, Ishikawa H, Yuasa N. Characterization of a heat-modifiable outer membrane protein of Haemophilus somnus. Infect. Immun. 1993; 61:1750-1755.

43. Sanders J D, Bastida-Corcuera F D, Arnold K F, Wunderlich A C, Corbeil L B. Genetic manipulation of immunoglobulin binding proteins of Haemophilus somnus. Microb. Pathog. 2003; 34:131-139.

44. Ward C K, Mock J R, Hansen E J. The LspB protein is involved in the secretion of the LspA1 and LspA2 proteins by Haemophilus ducreyi. Infect. Immun. 2004; 72:1874-1884.

45. Bakaletz L O, Barenkamp S J. Location of high molecular weight adhesion proteins of nontypeable Haemophilus influenzae by immunoelectson microscopy. Infect. Immun. 1994;. 62:4460-4468.

46. Barenkamp, S J. Immunization with high-molecular-weight adhesion proteins of nontypeable Haemophilus influenzae modifies experimental otitis media in chincillas. Infect. Immun. 1996;.64:1246-1251.

47. Delisse-Gathoye A-M, Locht C, Jacob F et al. Cloning, partial sequence, expression, and antigenic analysis of the filamentous hemagglutinin gene of Bordetella pertussis. Infect. Immun. 1990; 58:2895-2905.

48. Kajava A V, Cheng N, Cleaver R et al. Beta-helix model for the filamentous haemagglutinin adhesin of Bordetella pertussis and related bacterial secretory proteins. Mol. Microbiol. 2001; 42:279-292.

49. Makhov A M, Hannah J H, Brennan M J et al. Filamentous haemagglutinin of Bordetella pertussis. A bacterial adhesin formed as a 50-nm monomeric rigid rod based on a 19-residue repeat motif rich in beta strands and turns. J. Mol. Biol.1994; 241:110-124.

50. Fischetti V A. Streptococcal M protein: molecular design and biological behavior. Clin. Microbiol. Rev. 1989; 2:285-314.

51. Johnsson E, Andersson G., Lindahl G, Heden L-O. Identification of the IgA-binding region in streptococcal protein Arp. J. Immunol. 1994; 153:3557-3564.

52. Jerlström P G, Chhatwal G S, Timmis K N. The IgA-binding □-antigen of the c protein complex of Group B streptococci: sequence determination of its gene and detection of two binding regions. Mol. Microbiol. 1991; 5:843-849.

53. Gogolewski R P, Leathers C W, Liggit H D, Corbeil L B. Experimental Haemophilus somnus pneumonia in calves and immunoperoxidase localization of bacteria. Vet. Pathol. 1987; 24:250-256.

54. Ishibashi Y, Relman D A, Nishikawa A. Invasion of human respiratory epithelial cells by Bordetella pertussis: possible role for a filamentous hemagglutinin Arg-Gly-Asp sequence and ?5?1 integrin. Microb. Pathog. 2001; 30:279-288.

55. Brooks P C, Clark R A, Cheresh D A. Requirement of vascular integrin □v?3 for angiogenesis. Science 1994; 264:569-571.

56. Cheresh, D A. 1987. Human endothelial cells synthesize and express an Arg-Gly-Asp-directed adhesion. Proc. Natl. Acad. Sci. USA 84:6471-6475.

57. Coburn J, Magoun L, Bodary S C, Leong J M. Integrins □v□3 and □5□1 mediate attachment of lyme disease spirochetes to human cells. Infect. Immun. 1998; 66:1946-1952.

58. Menozzi F D, Mutombo R, Renauld G et al. Heparin-inhibitable lectin activity of the filamentous hemagglutinin adhesin of Bordetella pertussis. Infect. Immun. 1994; 62:769-778.

59. Springer T A. Adhesion receptors of the immune system. Nature 1990; 346:425-434.

60. Relman D, Tuomanen E, Falkow S, Golenbock D T, Saukkonen K, Wright S D. Recognition of a bacterial adhesin by an integrin: macrophage CR3 (□M□2, CD11b/CD18) binds filamentous hemagglutinin of Bordetella pertussis. Cell 1990; 61:1375-1382.

61. Saukkonen K, Cabellos C, Burroughs M, Prasad S, Tuomanen E. Integrin-mediated localization of Bordetella pertussis within macrophages: role in pulmonary colonization. J. Exp. Med. 1991; 173:1143-1149.

62. Gomis S M, Godson D L, Beskorwayne T, Wobeser G A, Potter M. Modulation of phagocytic function of bovine mononuclear phagocytes by Haemophilus somnus. Microb. Pathogen. 1997; 22:13-21.

63. Inzana T J, Corbeil L B. Development of a defined medium for Haemophilus somnus isolated from cattle. Am. J. Vet. Res. 1987; 48:363-369.

64. Sambrook J, Fritsch E F, Maniatis T. Molecular cloning: a laboratory manual. Cold Spring Harbor, N.Y. Cold Spring Harbor Laboratory, 1989.

65. Guan K-L, Dixon J E. Eukaryotic proteins expressed in Escherichia coli: an improved thrombin cleavage and purification procedure of fusion proteins with glutathione S-transferase. Anal. Biochem. 1991; 192:262-267.

66. Hirano H. Microsequence analysis of the winged bean seed proteins electroblotted from two-dimensional gel. J. Protein Chem. 1989; 8:115-130.

67. Tagawa Y, Ishikawa H, Yuasa N. Purification and partial characterization of the major outer membrane protein of Haemophilus somnus. Infect. Immun. 1993; 61:91-96.

68. Corbeil L B, Kania, S A, Gogolewski R P. Characterization of immunodominant surface antigens of Haemophilus somnus. Infect. Immun. 1991: 4295-4301.

69. Primal S V, Silva S, Little P B. The protective effect of vaccination against experimental pneumonia in cattle with Haemophilsu somnus outer membrane antigens and interference by lipopolysaccharide. Can. J. Vet. Res. 1990; 54:326-330.

What is claimed is:

1. A method for producing a protective immune response against a microbe in an animal, the method comprising:
administering an immune response activating amount of an isolated IbpA polypeptide to an animal in need thereof, whereby a protective immune response is produced in the animal.

2. A method according to claim 1, wherein the IbpA polypeptide comprises a sequence having SEQ ID NO: 2.

3. A method according to claim 1, wherein the polypeptide is an IbpA polypeptide variant or fragment.

4. A method according to claim 3, wherein the IbpA polypeptide variant or fragment comprises a sequence having at least about 70% sequence identity to SEQ ID NO: 2.

5. (canceled)

6. (canceled)

7. (canceled)

8. (canceled)

9. A method according to claim 3, wherein the IbpA polypeptide variant or fragment comprises an A5 fragment.

10. A method according to claim 3, wherein the IbpA polypeptide variant or fragment comprises a DR2 fragment.

11. (canceled)

12. (canceled)

13. (canceled)

14. (canceled)

15. A method according to claim 1, wherein the polypeptide is administered intraperitoneally, orally, inhalation, subcutaneously, intramuscularly, intranasally, topically, or intravenously.

16. A method according to claim 1, wherein the animal is selected from the group consisting of cattle, sheep, and bison.

17. (canceled)

18. A method according to claim 1, wherein the microbe is H. somni.

19. A method according to claim 1, wherein the microbe is selected from the group consisting of P. multocida, H. ducreyi, Bordetella species, Yersinia species, S. pyogenes, and S. agalactiae.

20. (canceled)

21. (canceled)

22. A method for producing a protective immune response against a microbe in an animal, the method comprising:
administering to a animal in need thereof an isolated polynucleotide encoding an IbpA polypeptide, wherein an immune response activating amount of the IbpA polypeptide is expressed in at least one animal cell,
whereby a protective immune response is produced in the subject.

23. A method according to claim 22, wherein the polynucleotide is comprised by a vector.

24. A method according to claim 22, wherein the polynucleotide encoding IbpA comprises a sequence having SEQ ID NO: 1.

25. A method according to claim 22, wherein the polynucleotide is an ibpA polynucleotide variant or fragment.

26. A method according to claim 25, wherein the ibpA polynucleotide variant or fragment comprises a sequence having at least about 70% sequence identity to SEQ ID NO: 1.

27. (canceled)

28. (canceled)

29. (canceled)

30. (canceled)

31. A method according to claim 25, wherein the ibpA polynucleotide variant or fragment comprises an A5 fragment.

32. A method according to claim 25, wherein the ibpA polynucleotide variant or fragment comprises a DR2 fragment.

33. A method according to claim 22, wherein the polynucleotide is administered intraperitoneally, orally, by inhalation, subcutaneously, intramuscularly, intranasally, topically, or intravenously.

34. A method according to claim 22, wherein the animal is selected from the group consisting of cattle, sheep, and bison.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. A vaccine comprising an IbpA polypeptide.

55. (canceled)

56. (canceled)

57. (canceled)

58. (canceled)

59. (canceled)
